(19) United States
(12) Patent Application Publication
(10) Pub. No.: US 2008/0213166 A1
(43) Pub. Date: Sep. 4, 2008

(54) Novel Gene Targets and Ligands that Bind Thereto for Treatment and Diagnosis of Colon Carcinomas

(75) Inventors: Karen McLachlan, Del Mar, CA (US); Dennis P. Gately, San Diego, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: BIOGEN IDEC INC., Cambridge, MA (US)

(21) Appl. No.: 10/535,458

(22) PCT Filed: Nov. 20, 2003
(86) PCT No.: PCT/US03/37206

§ 371 Date: Jun. 15, 2006

Related U.S. Application Data
(60) Provisional application No. 60/427,564, filed on Nov. 20, 2002.

(51) Int. Cl. A61K 51/10; C07H 21/00; C07K 16/18; C12Q 1/68; A61K 31/7105; A61K 31/711; A61K 38/00; G01N 33/53; C07K 2/00; A61K 39/395; A61P 35/00

(52) U.S. Cl. 424/1.49; 536/23.1; 536/24.33; 530/391.1; 530/300; 424/138.1; 435/6; 435/7.1; 435/7.93; 514/44; 514/2

(57) ABSTRACT

Isolated nucleic acids and proteins that are overexpressed in human cancers, and antibodies that specifically bind the proteins, which are useful diagnostic and therapeutic targets.

### RELATED APPLICATIONS

This application relates to PCT International Application No. PCT/US03/09534, filed Mar. 28, 2003, and to U.S. Provisional Patent Application No. 60/427,564 filed Nov. 20, 2002, each of which is incorporated by reference in its entirety herein.

### FIELD OF THE INVENTION

The present invention relates the identification of gene targets for treatment and diagnosis of neoplastic diseases, such as colon or colorectal cancer, and other cancers wherein the subject genes are upregulated and the use thereof to express the corresponding antigen, and to produce ligands that specifically bind such antigen, e.g. monoclonal antibodies and small molecules.

### DESCRIPTION OF RELATED ART

Colorectal cancers are among the most common cancers in men and women in the U.S. and are one of the leading causes of death. Other than surgical resection no other systemic or adjuvant therapy is available. Vogelstein and colleagues have described the sequence of genetic events that appear to be associated with the multistep process of colon cancer development in humans (Fearon and Vogelstein, 1990). An understanding of the molecular genetics of carcinogenesis, however, has not led to preventative or therapeutic measures. It can be expected that advances in molecular genetics will lead to better risk assessment and early diagnosis but colorectal cancers will remain a deadly disease for a majority of patients due to the lack of an adjuvant therapy.

Endogenous gastrins and exogenous gastrins (other than tetragastrin) seem to promote the growth of established colon cancers in mice (Singh, et al., 1986; Singh, et al., 1987; et al., 1984; Smith and Solomon, 1988; Singh, et al., 1990; Rehfeld and van Solinge, 1994) and promote carcinogen induced colon cancers in rats (Williamson et al., 1978; Karlin et al., 1985; Lamoste and Willems; 1988). Recent studies of Montag et al (1993) further support a possible co-carcinogenic role of gastrin in the initiation of tumors.

Many colon cancer cells express and secrete gastrin gene products (Dai et al., 1992; Kochinan et al., 1992; Finley et al., 1993; Van Solinge et al., 1993; Xu et al., 1994; Singh et al., 1994a; Hoosein et al., 1988; Hoosein et al., 1990) and bind gastrin-like peptides (Singh et al., 1986; Singh et al., 1987; Weinstock and Baldwin, 1988; Watson and Steele, 1994; Upp et al., 1989; Singh et al., 1985). In previous reports gastrin antibodies were either reported to inhibit (Hoosein et al., 1988; Hoosein et al, 1990) the growth of colon cancer cell lines in vitro.

However other investigators have had inconclusive results with colon cancer cell lines. A number of studies testing the effects of gastrin on cell proliferation of cancer cells have been performed (Sirinek et al., 1985; Kusyk et al., 1986; Watson et al., 1989). The results have varied widely. In one study, four different human cancer cell lines were tested for growth stimulation by pentagastrin and only one showed growth stimulation (Eggstein et al., 1991). Similarly in majority of the studies conducted to-date, mitogenic effects of gastrin have been demonstrated only on a very small percentage of colon cancer cell lines (Hoosein et al., 1988; Hoosein et al, 1990; Shrink et al, 1985; Kusyk et al, 1986; Guo et al, 1990; Ishizuka et al, 1994).

Since only a small percentage of established human colon cancer cell lines demonstrated a growth response to exogenous gastrins, investigators in this field came to believe that gastrin probably did not play a significant role in the growth of colon cancers. The recent discovery that human colon cancer cell lines and primary human colon cancers express the gastrin gene has sparked a renewed interest in a possible autocrine role of gastrin-like peptides in colon cancers. However, significant skepticism remains in the field, to date, regarding the importance of gastrin gene expression to the continued growth and tumorigenicity of colon cancers.

Thus, to-date, no systemic or adjuvant therapies have been developed for colon cancers, based on the knowledge that a significant percentage of human colon cancers express the gastrin gene. In fact, no adjuvant or systemic therapy has been developed for colon cancers that is based on the knowledge of the expression of other growth factors such as TGF-alpha. or IGF-II, since none of the growth factors demonstrate a significant growth effect on majority of the colon cancer cell lines in culture.

At the present time the only systemic treatment available for colon cancer is chemotherapy. However, chemotherapy has not proven to be very effective for the treatment of colon cancers for several reasons, in part because colon cancers express high levels of the MDR gene (that codes for multi-drug resistance gene products). The MDR gene products actively transport the toxic substances out of the cell before the chemotherapeutic agents can damage the DNA machinery of the cell. These toxic substances harm the normal cell populations more than they harm the colon cancer cells for the above reasons.

There is no effective systemic treatment for treating colon cancers other than surgically removing the cancers. In the case of several other cancers, including breast cancers, the knowledge of growth promoting factors (such as EGF, estradiol, IGF-II) that appear to be expressed or effect the growth of the cancer cells, has been translated for treatment purposes. But in the case of colon cancers this knowledge has not been applied and therefore the treatment outcome for colon cancers remains bleak.

Antisense RNA technology has been developed as an approach to inhibiting gene expression, including oncogene expression. An “antisense” RNA molecule is one which contains the complement of, and can therefore hybridize with, protein-encoding RNAs of the cell. It is believed that the hybridization of antisense RNA to its cellular RNA complement can prevent expression of the cellular RNA, perhaps by limiting its translatability. While various studies have involved the processing of RNA or direct introduction of antisense RNA oligonucleotides to cells for the inhibition of gene expression (Brown, et al., 1989; Wickstrom, et al., 1988; Smith, et al., 1986; Buvoli, et al., 1987), the more common means of cellular introduction of antisense RNAs has been through the construction of recombinant vectors that express antisense RNA once the vector is introduced into the cell.

A principle application of antisense RNA technology has been in connection with attempts to affect the expression of specific genes. For example, Delauney, et al. have reported the use antisense transcripts to inhibit gene expression in transgenic plants (Delauney, et al., 1988). These authors report the down-regulation of chloramphenicol acetyl transferase activity in tobacco plants transformed with CAT sequences through the application of antisense technology.

Antisense technology has also been applied in attempts to inhibit the expression of various oncogenes. For example, Kasid, et al., 1989, report the preparation of recombinant vector construct employing Craf-1 cDNA fragments in an antisense orientation, brought under the control of an adenovirus 2 late promoter. These authors report that the introduction of this recombinant construct into a human squamous carcinoma resulted in a greatly reduced tumorigenic potential relative to cells transfected faith control sense transfectants. Similarly, Prochownik, et al., 1988, have reported the use of Cmiyc antisense constructs to accelerate differentiation and inhibit G.sub.1 progression in Friend Murine Erythroleukemia cells. In contrast, Khokha, et al., 1989, discloses the use of antisense RNAs to confer oncogenicity on 3T3 cells, through the use of antisense RNA to reduce murine tissue inhibitor or metalloproteinases levels.

Antisense methodology takes advantage of the fact that nucleic acids tend to pair with “complementary” sequences. By complementary, it is meant that polynucleotides are those which are capable of base-pairing according to the standard Watson-Crick complementary rules. That is, the larger purines base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA leads to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, can be employed to inhibit gene transcription or translation or both within a host cell, either in vitro or in vivo, such as within a host animal, including a human subject.

Throughout this application, the term “expression vector or construct” is meant to include any type of genetic construct containing a nucleic acid coding for a gene product in which part or all of the nucleic acid encoding sequence is capable of being transcribed. The transcript can be translated into a protein but it need not be. Thus, in certain embodiments, expression includes both transcription of a gene and translation of mRNA into a gene product. In other embodiments, expression only includes transcription of the nucleic acid encoding a gene of interest.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter is used to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 base pairs of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 base pairs upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 base pairs apart before activity begins to decline. Depending on the promoter, it appears that individual elements can function either cooperatively or independently to activate transcription.

A promoter is selected based on its capability to direct gene expression in the targeted cell. Thus, where a human cell is targeted, the nucleic acid coding region can be positioned adjacent to and under the control of a promoter that is capable of being expressed in a human cell. Generally speaking, such a promoter might include either a human or viral promoter.

In various instances, the human cytomegalovirus (CMV) immediate early gene promoter, the SV40 early promoter and the Rous sarcoma virus long terminal repeat can be used to obtain high-level expression of the gene of interest. The use of other viral or mammalian cellular or bacterial phage promoters which are well known in the art to achieve expression of a gene of interest is contemplated as well, provided that the levels of expression are sufficient for a given purpose.

By employing a promoter with well-known properties, the level and pattern of expression of the gene product following transfection can be optimized. Further, selection of a promoter that is regulated in response to specific physiologic signals can permit inducible expression of the gene product. Representative elements/promoters useful in accordance with the present invention include but are not limited to those listed below.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. A promoter includes one or more elements that direct initiation of RNA synthesis at a particular site and in a particular orientation, whereas enhancers lack these specificities. Promoters and enhancers are often overlapping and contiguous, often seeming to have a very similar modular organization.

Viral promoters, cellular promoters/enhancers and inducible promoters/enhancers that could be used in combination with the nucleic acid encoding a gene of interest in an expression construct. Some examples of enhancers include Immunoglobulin Heavy Chain; Immunoglobulin Light Chain; T-Cell Receptor; HLA DQ a and DQ b b-Interferon; Interleukin-2; Interleukin-2 Receptor: Gibbon Ape Leukemia Virus; MHC Class II 5 or HLA-DRa; b-Actin; Muscle Creatine Kinase; Prealbumin (Transthyretin); Elastase I; Metallothionein; Collagenase, Albumin Gene; α-Fetoprotein; α-Globin; β-Globin; c-fos: c-HA-ras; Insulin Neural Cell Adhesion Molecule (NCAM); a1-Antitrypsin; H2B (TH2B) Histone; Mouse or Type I Collagen; Glucose-Regulated Proteins (GRP94 and GRP78); Rat Growth Hormone; Human Serum Amyloid A (SAA); Troponin I (TN I); Platelet-Derived Growth Factor; Duchenne Muscular Dystrophy; SV40 or CMV; Polyoma; Retroviruses; Papilloma Virus; Hepatitis B Virus; Human Immunodeficiency Virus. Inducers such as phorbol ester (TFA) heavy metals; glucocorticoids; poly (rl)X; poly(rc); Ela; H₂O₂; IL 1; Interferon, Newcastle Disease Virus; A23187; IL-6; Serum; SV40 Large T Antigen; FMA; thyroid Hormone; could be used. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

In certain instances, the expression construct can comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal et al., 1986: Temin, 1986). The first viruses used as gene vectors were DNA viruses including the papoviruses (simian virus 40, bovine papilloma virus, and polyoma) (Ridgeway, 1988; Baichwal et al., 1986) and adenoviruses (Ridgeway, 1988; Baichwal et al., 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Where a cDNA insert is employed, a polyadenylation signal is typically inserted to effect proper polyadenylation of the gene transcript. Any suitable polyadenylation sequence can be used. An expression cassette can also include a terminator sequence. These elements enhance message levels and minimize read through from the cassette into other sequences.

It is understood in the art that to bring a coding sequence under the control of a promoter, or operatively linking a sequence to a promoter, one positions the 5′ end of the transcription initiation site of the transcriptional reading frame of the protein between about land about 50 nucleotides “downstream” of (i.e., 3′ of) the chosen promoter. In addition, where eukaryotic expression is contemplated, an appropriate polyadenylation site (e.g., 5′-AATAAA-3′ (SEQ ID NO:66)) can be included if absent from the original cloned segment. Typically, the poly A addition site is placed about 30 to 2000 nucleotides “downstream” of the termination site of the protein at a position prior to transcription termination.

The above background references are part of the present invention insofar as they are applicable to the invention described herein. Hence there are no effective and specific ways of treating or diminishing the growth of colorectal cancer to date.

Therefore, there exists a significant need for the identification of novel gene targets for the treatment and diagnosis of colon or colorectal cancer, especially given the huge human toll caused by this disease annually.

### SUMMARY OF THE INVENTION

It is an aspect of the invention to identify gene targets for treatment and the diagnosis of cancer, including but not limited to cancer of the colon, pancreas, breast, ovary, and lung.

It is another aspect of the invention to provide the antigens expressed by genes that are expressed by malignant tissues, such as isolated protein antigens and isolated nucleic acids encoding the same.

It is another aspect of the invention to produce ligands that bind antigens expressed by certain cancers. Representative ligands include monoclonal antibodies.

It is another aspect of the invention to provide novel therapeutic regimens for the treatment of cancer that involve the administration of cancer antigens, alone or in combination with adjuvants that elicit an antigen-specific cytotoxic T-cell lymphocyte response against cancer cells that express such antigen.

It is another aspect of the invention to develop novel therapies for treatment of cancer involving the administration of anti-sense oligonucleotides corresponding to gene targets that are expressed by certain cancers.

It is another aspect of the invention to provide therapeutic regimens for the treatment of cancer that involve the administration of ligands, for example, monoclonal antibodies, peptides, and small molecules that specifically bind the disclosed cancer antigens.

It is another aspect of the invention to provide methods for diagnosis of cancer using ligands, e.g., monoclonal antibodies, that specifically bind to antigens that are expressed by cancers in order to detect whether a subject has cancer or is at increased risk of developing cancer.

It is another aspect of the invention to provide methods for detecting persons having, or at increased risk of developing certain types of cancers using labeled nucleic acids that hybridize to the disclosed nucleic acids that encode cancer antigens.

It is yet another aspect of the invention to provide diagnostic test kits for the detection of persons having or at increased risk of developing certain cancer. For example, diagnostic kits of the invention can comprise a ligand that specifically binds to a cancer antigen and a detectable label, e.g., a radiolabel or fluorophore. A diagnostic kit of the invention can also comprise a nucleic acid, including for example, PCR primers, of a cancer antigen and a detectable label.

### BRIEF DESCRIPTION OF THE DRAWINGS

FIG. 1 summarizes expression data for the CICO1, CICO2 and CICO3, which were identified based on overexpression in colon cancer as described in Example 1.

FIGS. 2-5 depict gene expression profiles determined using the GENE LOGIC® datasuite as described in Example 2. The values along the y-axis represent expression intensities in Gene Logic units. Each circle represents an individual patient sample. The bar graph on the left of the figure depicts the percentage of each tissue type found to express the gene fragment. The total number of samples for each tissue type is as follows: colon tumor, tumor % above 50, 31; colon tumors, 45; normal breast, 37; normal colon, 30; normal esophagus, 18, normal kidney, 28; normal liver, 21; normal lung, 35; normal lymph node 10; normal ovary, 25; normal pancreas, 20; normal prostate, 20; normal rectum, 22; normal stomach, 25. “Colon tumor, tumor % above 50” refers to tumor samples for which at least 50% of each sample comprises malignant tissue, as determined by a pathologist. This sample set is a subset of colon tumors, which comprises all colon tumor samples contained within the GENE LOGIC® database.

FIG. 2 depicts the gene expression profile of Candidate 1, which was determined using the GENE LOGIC® datasuite for GenBank Accession No. W91975 as described in Example 2. Candidate 1 is overexpressed in colon tumor tissue.

FIG. 3 depicts the gene expression profile of Candidate 2, which was determined using the GENE LOGIC® datasuite for GenBank Accession No. AI694242 as described in Example 2. Candidate 2 is overexpressed in colon tumor tissue.

FIG. 4 contains the gene expression profile of Candidate 3, which was determined using the GENE LOGIC® datasuite for GenBank Accession No. AI680111 as described in Example 2. Candidate 3 is overexpressed in colon tumor tissue.

FIG. 5 depicts the gene expression profile of Candidate 4, which was determined using the GENE LOGIC® datasuite for GenBank Accession No. AA813827 as described in Example 2. Candidate 4 is overexpressed in colon tumor tissue.

FIGS. 6A and 6B show PCR data of Candidate 3 expression (FIG. 6A) and GAPDH expression (FIG. 6B) in normal human tissues. Candidate 3 was screened against Human Multiple Tissue cDNA panels I & II (Clontech #K1420-1 & # K1421-1) according to the manufacturer's instructions. GAPDH was not tested against the prostate sample. The positive control for Candidate 3 was IMAGE 2324560, obtained from the American Tissue Type Collection (Manassas, Va.). The cDNA samples present in each lane are as follows: lane 1, heart; lane 2, brain; lane 3, placenta; lane 4, lung; lane 5, liver; lane 6, skeletal muscle; lane 7, kidney; lane 8, pancreas; lane 9, spleen; lane 10, thymus; lane 11, prostate; lane 12, testis; lane 13, ovary; lane 14, small intestine; lane 15, colon; lane 16, peripheral blood leukocytes; lane 17, positive control; lane 18, negative control. Arrow denotes the anticipated size of the PCR product for candidate 3. The results shown in this figure indicate that candidate 3 is not expressed at detectable levels in any of the normal tissues tested.

FIGS. 7A and 7B show PCR data of Candidate 3 expression (FIG. 7A) and GAPDH expression (FIG. 7B) in colon tumor samples. The cDNA samples present in each lane are as follows: lane 1, grade 3 adenocarcinoma; lane 2, grade 2 adenocarcinoma; lane 3, grade 1 adenocarcinoma; lane 4, grade 2 adenocarcinoma; lane 5, colorectal cancer cell line HCT116; lane 6, positive control (IMAGE clone); lane 7, negative control. Arrow denotes the anticipated size of the PCR product for candidate 3. The results shown in this figure indicate that candidate 3 is expressed in at least 3 of 4 colon tumor samples in addition to colorectal tumor cell line HCT116.

FIG. 8 depicts E-Northern expression data for Loc 56926, which is overexpressed in colon cancer, as described in Example 4. The values along the y-axis represent expression intensities in Gene Logic units. Each circle on the figure represents an individual patient sample. The bar graph on the left of the figure depicts the percentage of each tissue type found to express the gene fragment. The total number of samples for each tissue type found to express the gene fragment. The total number of samples for each tissue type is indicated in the legend to the left of the bar graph. The designation “50%” for malignant samples refers to the fact that the tumor samples contain greater than 50% tumor material as determined by a certified pathologist.

FIGS. 9A and 9B are PCR panels showing expression of Loc56926 (FIG. 9A) and GAPDH (FIG. 9B) in malignant colon samples. The cDNA samples present in each lane are as follows: lane M, marker; lane 1, no template control; lane 2 colon cancer 8T; lane 3, colon cancer DT; lane 4, colon cancer FT; lane 5, colon cancer GT; lane 6, colon cancer HT; lane 7, colon cancer IT; lane 8, colon cancer QT; lane 9, prostate cancer OT; lane 10, colon cancer RT; lane 11, colon cancer cell line HCT116; lane 12, positive control EST. The results from this figure demonstrate that Loc56926 expression is present in cDNA from three of eight tested colon cancer samples.

FIGS. 10A and 10B are PCR panels showing expression of Loc56926 (FIG. 10A) and GAPDH (FIG. 10B) in normal human tissues. Hybridization was performed using Human Multiple Tissue cDNA panel I (Clontech #K1420-1) according to the manufacturer's instructions. The cDNA samples present in each lane are as follows: lane M, marker; lane 1, no template control; lane 2, colon tumor 8T; lane 3, colon tumor HT; lane 4, colon tumor RT; lane 5, colon cancer cell line HCT116; lane 6, normal colon; lane 7, normal brain; lane 8, normal heart; lane 9, kidney; lane 10, normal liver; lane 11, normal lung; lane 12, skeletal muscle; lane 13, normal pancreas; lane 14, normal placenta lane 15; EST control. These results demonstrate that Loc56926 is present in colon tumors with light expression in the normal pancreas (note the increase in GAPDH in the pancreas lane compared to the colon tumor lanes) and not expressed at detectable levels the other tested normal human tissues.

FIGS. 11A and 11B are PCR panels showing expression of Loc56926 (FIG. 11A) and GAPDH (FIG. 11B) in human tissues. Hybridization was performed using Human Multiple Tissue cDNA panel II (Clontech # K1421-1) according to the manufacturer's instructions. The cDNA samples present in each lane are as follows: lane M, marker; lane 1, no template control; lane 2, colon tumor 8T; lane 3, colon tumor HT; lane 4, colon tumor RT; lane 5, colon cancer cell line HCT116; lane 6, normal colon; lane 7, normal peripheral blood leukocytes; lane 8, small intestine; lane 9, normal ovary; lane 10, normal prostate; lane 11, normal spleen; lane 12, normal testis; lane 13, normal thymus; lane 14, EST control. These results demonstrate that Loc56926 is not expressed at detectable levels in these normal tissues.

FIGS. 12A and 12B are PCR panels showing expression of Loc56926 (FIG. 12A) and GAPDH (FIG. 12B) in normal brain tissue samples. Hybridization was performed using Normal Neural System cDNA panel (Biochain, C8234503, C8234504, C8234505). The cDNA samples present in each lane are as follows: lane M, marker; lane 1, no template control; lane 2, cerebellum; lane 3, cerebral cortex; lane 4, medulla oblongata; lane 5, pons; lane 6, frontal lobe; lane 7, occipital lobe; lane 8, parietal lobe; lane 9, temporal lobe; lane 10, placental neural system; lane 11, EST control. These results demonstrate that Lco56926 is not expressed at detectable levels in the normal brain.

FIGS. 13-19 depict E-Northern expression data for genes detected at elevated levels in malignant colon tissues as well as other cancers. Each circle on the figure represents an individual patient sample. The bar graph on the left of the figure depicts the percentage of each tissue type found to express the gene fragment. The total number of samples for each tissue type found to express the gene fragment. The total number of samples for each tissue type is indicated in the legend to the left of the bar graph. The designation “50%” for malignant samples refers to the fact that the tumor samples contain greater than 50% tumor material as determined by a certified pathologist.

FIG. 13 depicts E-Northern expression data for the AW779536 gene, which is overexpressed in colon cancer, as described in Example 4.

FIG. 14 depicts E-Northern expression data for the AL531683 gene, which is overexpressed in colon cancer, as described in Example 4.

FIG. 15 depicts E-Northern expression data for the AI202201 gene, which is overexpressed in colon cancer, as described in Example 4.

FIG. 16 depicts E-Northern expression data for the AL389942 gene, which is overexpressed in colon cancer, as described in Example 4.

FIG. 17 depicts E-Northern expression results for the Ly6G6Dgene, also described in Example 5.

FIG. 18 depicts E-Northern expression results for FLJ32334, also described in Example 6.

FIG. 19 depicts E-Northern expression results for FLJ300002, also described in Example 7.

FIGS. 20A and 20B are PCR panels showing expression of CHEM1 (FIG. 20A) and GAPDH (FIG. 20B) in normal and tumor tissue samples (panel I). The cDNA samples (1 ng/lane) present in each lane were as follows: lane M, marker DNA; lane 1, no cDNA; lane 2, prostate tumor N; lane 3, prostate tumor O; lane 4, prostate tumor T; lane 5, colon tumor f; lane 6, colon tumor G; lane 7, colon tumor R; lane 8, normal brain; lane 9, normal colon; lane 10, normal heart; lane 11, normal kidney; lane 12, normal liver; lane 13, normal lung; lane 14, normal skeletal muscle; lane 15, normal pancreas; lane 16, normal placenta; lane 17, normal prostate; lane 18, normal thymus.

FIGS. 21A and 21B are PCR panels showing expression of CHEM1 (FIG. 21A) and GAPDH (FIG. 21B) in normal and tumor tissue samples (panel I). The cDNA samples (5 ng/lane) present in each lane were as follows: lane M, marker DNA; lane 1, no cDNA; lane 2, prostate tumor N; lane 3, prostate tumor O; lane 4, colon tumor f; lane 5, colon tumor G; lane 6, colon tumor R; lane 7, normal brain; lane 8, normal colon; lane 9, normal heart; lane 10, normal kidney; lane 11, normal liver; lane 12, normal lung; lane 13, normal skeletal muscle; lane 14, normal pancreas; lane 15, normal placenta; lane 16, normal prostate; lane 17, normal thymus.

FIGS. 22A and 22B are PCR panels showing expression of CHEM1 (FIG. 22A) and GAPDH (FIG. 22B) in normal and tumor tissue samples (panel II). The cDNA samples (5 ng/lane) present in each lane were as follows: lane M, marker DNA; lane 1, no cDNA; lane 2, prostate tumor N; lane 3, colon tumor R; lane 4, normal colon; lane 5, normal heart; lane 6, normal peripheral blood lymphocytes; lane 7, normal small intestine; lane 8, normal ovary; lane 9, normal spleen; lane 10, normal testis; lane 11, normal thymus.

FIGS. 23A and 23B are PCR panels showing expression of CHEM1 (FIG. 23A) and GAPDH (FIG. 23B) in normal brain and tumor tissue samples. The cDNA samples (5 ng/lane) present in each lane are as follows: lane M, marker DNA; lane 1, no cDNA; lane 2, prostate tumor N; lane 3, prostate tumor O; lane 4, colon tumor R; lane 5, cerebral cortex; lane 6, cerebellum; lane 7, medulla oblongata; lane 8, pons; lane 9, frontal lobe; lane 10, occipital lobe; lane 11, parietal lobe; lane 12, temporal lobe; lane 13, placenta.

FIGS. 24A and 24B are PCR panels showing expression of CHEM1 (FIG. 24A) and GAPDH (FIG. 24B) in normal heart and tumor tissue samples. The cDNA samples (5 ng/lane) present in each lane were as follows: lane M, marker DNA; lane 1, no cDNA; lane 2, prostate tumor N; lane 3, colon tumor R; lane 4, adult heart; lane 5, fetal heart; lane 6, aorta; lane 7, apex; lane 8, left atrium; lane 9, right atrium; lane 10, left ventricle; lane 11, right ventricle; lane 12, dextra auricle; lane 13, sinistra auricle; lane 14, atrioventricular node; lane 15, septum intraven.

FIG. 25 is a bar graph showing the results of a TAQMAN® assay performed using the indicated tissues.

FIGS. 26A and 26B are PCR panels showing expression of CHEM1 (FIG. 26A) and GAPDH (FIG. 26B) in samples prepared from human tumor cell lines. The cDNA samples present in each lane were as follows: lane 1, NCI-H2126 (lung); lane 2, SW620 (colon); lane 3, ZR-75-1 (breast); lane 4, MDA-MB-468 (breast); lane 5, UACC326 (ovary); lane 6, UACC812 (breast); lane 7, ME-180 (breast); lane 8, MDA-MB-231 (breast); lane 9, HT29 (colon); lane 10, A549 (lung); lane 11, LoVo (colon); lane 12, PANC-1 (pancreas); lane 13, NCI-H69 (lung); lane 14, NCI-H1299 (lung); lane 15, Colo 201 (colon); lane 16, Colo 205 (colon); lane 17, Colo 320 (colon); lane 18, negative control; lane 19, positive control.

FIG. 27 is a Western blot showing detection of CHEM1 protein in samples prepared from human tumor cell lines. The protein extracts (50 μg) present in each lane were as follows: lane 1, NCI-H69 (lung); lane 2, ZR-75-1 (breast); lane 3, MDA-MB-468 (breast); lane 4, AsPC-1; lane 5, HT-29 (colon); lane 6, LS 174T; lane 7, HCT 116.

FIG. 28 is a Western blot showing detection of CHEM1 protein cultured MDA-MB-468 or ZR-75-1 human tumor cell lines. The protein extracts (50 μg) present in each lane were as follows: lanes 1 and 4, post-nuclear supernatant (PNS); lanes 2 and 5, cytosol; lanes 3 and 6, membrane.

### DETAILED DESCRIPTION OF THE INVENTION

The present invention relates to the identification of genes which are to be specifically expressed and upregulated in certain cancers, including colon or colorectal tumors. This was determined using the GENE LOGIC® (Gaithersburg, Md.) datasuite or Celera (Rockville, Md.) database and by screening malignant colon tumor tissues as described in detail herein.

In particular, the present invention involves the discovery that certain genes, the nucleic acid sequences and predicted coding sequences of which are identified herein are specifically expressed in certain malignant tissues including colon or colorectal tumor tissues.

The disclosed therapies involve the synthesis of oligonucleotides having sequences in the antisense orientation relative to the genes identified by the present inventors which are specifically expressed by malignant tissues, including colon or colorectal tumors. Suitable therapeutic antisense oligonucleotides typically vary in length from two to several hundred nucleotides in length, more typically about 50-70 nucleotides in length. These antisense oligonucleotides can be administered as naked DNAs or in protected forms, e.g., encapsulated in liposomes. The use of liposomal or other protected forms may enhance in vivo stability and delivery to target sites, i.e., colon tumor cells.

Also, the subject novel genes can be used to design novel ribozymes that target the cleavage of the corresponding mRNAs in colon and other tumor cells. Similarly, these ribozymes can be administered in free (naked) form or by the use of delivery systems that enhance stability and/or targeting, e.g., liposomes. Ribozymal and antisense therapies used to target genes that are selectively expressed by cancer cells are well known in the art.

Also, the present invention embraces the administration of use of DNAs that hybridize to the novel gene targets identified herein, attached to therapeutic effector moieties, for example radiolabels, including metallic and halogen isotopes (e.g., ⁹⁰yttrium, ¹³¹iodine), cytotoxins, cytotoxic enzymes, in order to selectively target and kill cells that express these genes, i.e., colon tumor cells.

Still further, the present invention encompasses non-nucleic acid based therapies, for example antigens encoded by the nucleic acids disclosed herein. It is anticipated that these antigens can be used as therapeutic or prophylactic anti-tumor vaccines. For example, antigens of the present invention can be administered with adjuvants that induce a cytotoxic T lymphocyte response. Representative adjuvants include those disclosed in U.S. Pat. Nos. 5,709,860, 5,695,770, and 5,585,103, which promote CTL responses against prostate and papillomavirus related human colon cancer. The disclosures of U.S. Pat. Nos. 5,709,860, 5,695,770, and 5,585,103 are incorporated by reference in their entirety.

The disclosed antigens can be administered in combination with an adjuvant to elicit a humoral immune response against such antigens, thereby delaying or preventing the development of cancers (e.g., a colon cancer) associated with the overexpression of the antigens.

Embodiments of the invention comprise administration of one or more novel-colon cancer antigens, for example in combination with an adjuvant. A representative adjuvant is PROVAX®, which comprises a microfluidized adjuvant containing Squalene, TWEEN® and PLURONIC®, in an amount sufficient to be therapeutically or prophylactically effective. See U.S. Pat. Nos. 5,709,860, 5,695,770, and 5,585,103. A typical dosage of formulated antigen ranges from about 50 to about 20,000 mg/kg body weight, or from about 100 to about 5000 mg/kg body weight.

Alternatively, the subject tumor-associated antigens can be administered with other adjuvants, e.g., ISCOM®, DETOX™, SAF®, Freund's adjuvant, Alum, Saponin, among others.

In another embodiment, the present invention provides methods for preparing monoclonal antibodies against the antigens encoded by the DNA sequences disclosed in the examples which are expressed specifically by certain malignant tissues including colon or colorectal tumor tissues. Monoclonal antibodies are produced by conventional methods and include human monoclonal antibodies, humanized monoclonal antibodies, chimeric monoclonal antibodies, single chain antibodies, including scFv's and antigen-binding antibody fragments such as Fabs, 2 Fabs, and Fab′ fragments. Methods for the preparation of monoclonal antibodies and fragments thereof, for example by pepsin or papain-mediated cleavage, are well known in the art. In general, an appropriate (non-homologous) host is immunized with the subject colon cancer antigens, immune cells are isolated from the host and used to prepare hybridomas. Monoclonal antibodies that specifically bind to either of such antigens are identified by routine screening techniques. Useful monoclonal antibodies typically bind the target antigens with high affinity, e.g., possess a binding affinity (Kd) on the order of 10⁻⁶ to 10⁻¹⁰ M.

As used herein, the term “antibody” includes antigen-binding fragments and variants of the disclosed antibodies. Antibodies of the invention are readily modified wherein one or more of the constant region domains has been deleted or otherwise altered so as to provide desired biochemical characteristics. For example, modified antibodies having at least a portion of one of the constant domains deleted are referred to as “domain deleted” antibodies. See e.g., U.S. patent application Ser. Nos. 10/058,120 and 60/483,877 and PCT International Patent Publication No. WO 02/60955, each incorporated herein in its entirety. Representative domain deleted antibodies include antibodies that lack an entire constant region domain, such as an entire C_{H}2 domain. The omitted constant region domain can be replaced by a short amino acid spacer (e.g., 10 residues) that provides some of the molecular flexibility typically imparted by the absent constant region.

The domain structures and three dimensional configuration of the constant regions of the various immunoglobulin classes are well known. For example, the C_{H}2 domain of a human IgG Fc region usually extends from about residue 231 to residue 340 using conventional numbering schemes. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 10⁸ residues while the hinge region of an IgG molecule joins the C_{H}2 domain with the C_{H}1 domain. This hinge region encompasses on the order of 25 residues and is flexible, thereby allowing the two N-terminal antigen binding regions to move independently.

It is also known in the art that the constant regions mediate several effector functions. For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. Although various Fc receptors and receptor sites have been studied to a certain extent, there is still much which is unknown about their location, structure and functioning. Thus, the antibodies disclosed herein can be modified to alter physiological profile, bioavailability, and other biochemical effects, which altered traits are easily be measured and quantified using well known immunology techniques without undue experimentation.

Antibodies of the invention are useful for anti-tumor immunotherapy. Optionally, therapeutic effector moieties (e.g., radiolabels, cytotoxins, therapeutic enzymes, agents that induce apoptosis) can be attached to the antibodies to provide for targeted cytotoxicity, i.e., killing of human colon tumor cells. Given the fact that the subject genes are apparently not significantly expressed by many normal tissues this should not result in significant adverse side effects (toxicity to non-target tissues).

Antibodies and/or antibody fragments are administered to a subject in labeled or unlabeled form, alone or in combination with other therapeutics, such as chemotherapeutics such as progestin, EGFR, TAXOL®, and the like. The administered composition can include a pharmaceutically acceptable carrier, and optionally adjuvants, stabilizers, etc., used in antibody compositions for therapeutic use.

The present invention also provides diagnostic methods for detection of the colon or colorectal tumor-specific genes disclosed herein. Diagnostic methods include detecting the expression of one or more of these genes at the DNA level or at the protein level. Patients who test positive for the disclosed tumor-specific genes diagnosed are identified as having or being at increased risk of developing colon cancer. Additionally, the levels of antigen expression can be useful in determining patient status, i.e., how far the disease has advanced. For example, the expression or expression level of a tumor-specific gene can indicate a particular stage of tumor progression.

At the DNA level, gene expression is detected by known DNA detection methods, including but not limited to Northern blot hybridization, strand displacement amplification (SDA), catalytic hybridization amplification (CHA), PCR amplification (for example, using primers corresponding to the novel genes disclosed herein), and other known DNA detection methods. For example, the presence or absence of cancer associated with the genes disclosed herein can be determined based on whether PCR products are obtained, and the level of expression. Expression levels can also be monitored to determine the prognosis of a colon cancer patient as the levels of expression of the PCR product likely increase as the disease progresses. Suitable controls and quantification is are performed for diagnostic methods as known in the art.

At the protein level, the status of a subject to be tested for colon cancer, or other cancer associated by overexpression of a gene disclosed herein, can be evaluated by testing biological fluids, such as blood, urine, colon tissue, with an antibody or antibodies or fragment that specifically binds to the novel colon tumor antigens disclosed herein. Methods of using antibodies to detect antigen expression are well known and include ELISA, competitive binding assays, and the like. Representative assays use an antibody or antibody fragment that specifically binds the target antigen directly or indirectly bound to a label that provides for detection, for example, a radiolabel, an enzyme, or a fluorophore.

As noted, the present invention provides novel genes and corresponding antigens that correlate to human colon cancer. The present invention also embraces variants thereof. By “variants” is intended sequences that are at least 75% identical thereto, for example at least 85% identical, or at least 90% identical when these DNA sequences are aligned to the subject DNAs or a fragment thereof having a size of at least 50 nucleotides. Representative variants include allelic variants.

The present invention also provides primers for amplification of nucleic acids encoding the subject novel genes or a portion thereof, which are present is a biological sample, for example, an mRNA library obtained from a desired cell source, including human colon cell or tissue samples. Typically, such primers are about 12 to 50 nucleotides in length and are constructed such that they provide for amplification of the entire or most of the target gene.

The present invention further provides antigens encoded by the disclosed DNAs or fragments thereof that bind to or elicit antibodies specific to the full-length antigens. Typically, such fragments are at least 10 amino acids in length, more typically at least 25 amino acids in length.

The colon or colorectal tumor-specific genes of the invention are expressed in a majority of colon tumor samples tested. Some of these genes are also upregulated in other cancers. Thus, the present invention further contemplates identification of other cancers wherein the expression of the disclosed genes or variants thereof correlate to a cancer or an increased likelihood of cancer, for example breast, pancreas, lung or colon cancers. Also provided are compositions and methods to detect and treat such cancers.

“Isolated” refers to any human protein that is not in its normal cellular millieu. This includes by way of example compositions comprising recombinant protein, pharmaceutical compositions comprising purified protein, diagnostic compositions comprising purified protein, and isolated protein compositions comprising protein. In representative embodiments of the invention, an isolated protein comprises a substantially pure protein, in that it is substantially free of other proteins, for example, at least 90% pure, that comprises the amino acid sequence disclosed herein or natural homologues or mutants having essentially the same sequence. A naturally occurring mutant might be found, for instance, in tumor cells expressing a gene encoding a mutated protein sequence.

“Native human protein” refers to a protein that comprises the amino acid sequence of the protein expressed in its endogenous environment, i.e., a human colon or colorectal tumor tissue.

“Native non-human primate protein” refers to a protein that is a non-human primate homologue of the protein having the amino acid sequence discussed in the examples. Given the phylogenetic closeness of humans to other primates, it is anticipated that human and non-human proteins expressed by the genes disclosed in the examples have non-human primate counterparts that possess amino acid sequences that are highly similar, such as 95% sequence identity or higher.

“Isolated human or non-human primate nucleic acid molecule or sequence” refers to a nucleic acid molecule that encodes human protein which is not in its normal human cellular millieu, e.g., is not comprised in the human or non-human primate chromosomal DNA. This includes by way of example vectors that comprise a nucleic acid molecule, a probe that comprises a gene nucleic acid sequence directly or indirectly attached to a detectable moiety, e.g. a fluorescent or radioactive label, or a DNA fusion that comprises a nucleic acid molecule encoding a colon antigen according to the invention fused at its 5′ or 3′ end to a different DNA, e.g. a promoter or a DNA encoding a detectable marker or effector moiety. Representative nucleic acid sequence encoding human proteins are disclosed herein. Also included are natural homologues or mutants having substantially the same sequence. Naturally occurring homologies that are degenerate would encode the same protein as discussed herein in the examples, but would include nucleotide differences that do not change the corresponding amino acid sequence. Naturally occurring mutants might be found in tumor cells, wherein such nucleotide differences result in a mutant protein. Naturally occurring homologues containing conservative substitutions are also encompassed.

“Variant of human or non-human primate protein” refers to a protein possessing an amino acid sequence that possess at least 90% sequence identity, such as at least 91% sequence identity, or at least 92% sequence identity, or at least 93% sequence identity, or at least 94% sequence identity, or at least 95% sequence identity, or at least 96% sequence identity, or at least 97% sequence identity, or at least 98% sequence identity, and including at least 99% sequence identity, to the corresponding native human or non-human primate protein wherein sequence identity is as defined herein. Preferably, a variant possesses at least one biological property in common with the human or non-human protein.

“Variant of human or non-human primate nucleic acid molecule or sequence” refers to a nucleic acid sequence that possesses at least 90% sequence identity, such as at least 91%, or at least 92%, or at least 93%, or at least 94%, or at least 95%, or at least 96%, or at least 97%, or at least 98% sequence identity, and including at least 99% sequence identity, to the corresponding native human or non-human primate nucleic acid sequence, wherein “sequence identity” is as defined herein.

“Fragment of human or non-human primate nucleic acid molecule or sequence” refers to a nucleic acid sequence corresponding to a portion of the native human nucleic acid sequence discussed herein in the examples or a primate native non-human homolog molecule, wherein said portion is at least about 50 nucleotides in length, or 100, for example, at least 200 or 300 nucleotides in length.

“Antigenic fragments of colon or colorectal” refer to polypeptides corresponding to a fragment of colon antigen encoded by any of the genes disclosed herein or a variant or homologue thereof that when used itself or attached to an immunogenic carrier that elicits antibodies that specifically bind the protein. Typically, antigenic fragments are at least 20 amino acids in length.

Sequence identity or percent identity is intended to mean the percentage of the same residues shared between two sequences, referenced to the human DNA or amino acid sequences disclosed herein, when the two sequences are aligned using the Clustal method [Higgins et al, Cabios 8:189-191 (1992)] of multiple sequence alignment in the Lasergene biocomputing software (DNASTAR, INC. of Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignmen=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM25O [Dayhoff et al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to a human gene of the invention when determining percent conservation with a non-human gene and when determining percent conservation. Conservative amino acid changes satisfying this requirement include: R-K; E-D, Y-F, L-M; V-I, Q-H.

### Polypeptide Fragments

The invention provides polypeptide fragments of the disclosed proteins. Polypeptide fragments of the invention can comprise at least 8 amino acid residues, such as at least 25 or at least 50 amino acid residues of human or non-human primate gene according to the invention or an analogue thereof. Polypeptide fragments can also comprise at least 75, 100, 125, 150, 175, 200, 225, 250, or 275 residues of the polypeptide encoded by gene the subject genes which are specifically expressed by certain human colon or colorectal as well as some other tumor tissues. In one embodiment of the invention, a protein fragment can also comprise a majority of the native protein colon or colorectal protein, i.e. at least about 100 contiguous residues of the native colon or colorectal protein antigen.

### Biologically Active Variants

The invention also encompasses biologically active mutants of protein colon or colorectal proteins according to the invention, which comprise an amino acid sequence that is at least 80%, for example, 90% or 95-99% similar to the subject tumor-associated proteins.

Guidance in determining which amino acid residues can be substituted, inserted, or deleted without abolishing biological or immunological activity can be found using computer programs well known in the art, such as DNASTAR software. Protein variants can include conoservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid can be made without affecting the biological properties of the resulting secreted protein or polypeptide variant.

Human or non-human primate protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the protein gene are also variants. Covalent variants can be prepared by linking functionalities to groups which are found in the amino acid chain or at the N- or C-terminal residue, as is known in the art.

Some amino acid sequence of the proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. Numerous substitutions at non-critical regions of the protein are well tolerated. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention can include one or more amino acid substitutions, deletions or additions, either from natural mutations or human manipulation.

The invention further includes variations of the protein subject colon or colorectal which show comparable expression patterns or which include antigenic regions. Protein mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

For example, charged amino acids can be substituted with another charged amino acid, or with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J Mol. Biol. 224:899-904 (1992) and de Vos et al. Science 255: 306-312 (1992)).

Conservative amino acid substitutions often do not significantly affect the folding or activity of the protein. A skilled artisan could determine an appropriate number and nature of amino acid substitutions based on factors as described above. Generally speaking, the number of substitutions for any given polypeptide are fewer than 50, 40, 30, 25, 20, 15, 10, 5 or 3 residues.

### Fusion Proteins

Fusion proteins comprising proteins or polypeptide fragments of the subject colon or colorectal proteins can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. The foregoing can also be adapted as a screening technique. Fusion proteins comprising a signal sequence and/or a transmembrane domain of a protein according to the invention or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize any of the amino acid sequences or encoded by the nucleotide sequences disclosed herein, or can be prepared from biologically active variants or fragment of said protein sequence, such as those described above. The first protein segment can consist of a full-length protein or a variant or fragment thereof. These fragments can range in size from about 8 amino acids up to the full length of the protein.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding an amino acid sequence according to the invention in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a sequence listing encoding one of the subject colon or colorectal proteins can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

Proteins can be further modified, for example by phosphorylation or glycosylation of the appropriate sites, in order to obtain a functional protein. Covalent attachments can be made using known chemical or enzymatic methods.

Human or non-human primate proteins according to the invention or polypeptide of the invention can also be expressed in cultured host cells in a form that facilitates purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% base pair mismatches. Homologous nucleic acids can contain 15-25% base pair mismatches or fewer, for example about 5-15% base pair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the gene A and gene B nucleic acid sequences provided herein. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers based on the disclosed sequences for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising human or non-human primate gene promoter and UTR sequences, operably linked to either protein coding sequences or other sequences encoding a detectable or selectable marker. Promoter and/or UTR-based constructs are useful for studying the transcriptional and translational regulation of protein expression, and for identifying activating and/or inhibitory regulatory proteins.

### Host Cells

An expression construct can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacteriol 153: 163 (1983); Kurtz et al., Mol. Cell Biol. 6: 142 (1986); Kunze et al., J Basic Microbiol. 25: 141 (1985); Gleeson et al., J. Gen. Microbiol. 132: 3459 (1986), Roggenkamp et al., Mol. Gen. Genet. 202: 302 (1986)); Das et al. J Bacteriol. 158: 1165 (1984); De Louvencourt et al., J Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8, pp. 277-279 (Plenum Publishing, 1986); and Maeda et al, Nature, 315: 592-594 (1985).

Mammalian expression can be accomplished as described in Dijkema et al. EMBO J. 4: 761 (1985); Gorman et al., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides of the nucleotide sequences disclosed herein. The transcription unit is located upstream to a coding sequence of the endogenous gene. The exogenous regulatory sequence directs transcription of the coding sequence of the endogenous gene.

Human or non-human primate protein can also include hybrid and modified forms thereof including fusion proteins, fragments and hybrid and modified forms in which certain amino acids have been deleted or replaced, modifications such as where one or more amino acids have been changed to a modified amino acid or unusual amino acid.

Also included within the meaning of substantially homologous is any human or non-human primate protein which shows cross-reactivity with antibodies to a gene described herein or whose encoding nucleotide sequences including genomic DNA, mRNA or cDNA are isolated through hybridization with the complementary sequence of genomic or subgenomic nucleotide sequences or cDNA of a gene disclosed herein or a fragment thereof. Degenerate DNA sequences that encode human or non-human primate proteins are also included within the present invention as are allelic variants of.

Colon or colorectal proteins of the invention can be prepared using recombinant DNA techniques. By “pure form” or “purified form” or “substantially purified form” it is meant that a protein composition is substantially free of other proteins which are not protein.

The present invention also includes therapeutic or pharmaceutical compositions comprising human or non-human primate proteins, fragments or variants according to the invention in an effective amount for treating patients with disease, and a method comprising administering a therapeutically effective amount of a protein according to the invention. These compositions and methods are useful for treating cancers associated with a protein according to the invention, e.g. colon cancer. One skilled in the art can readily use a variety of assays known in the art to determine whether a protein according to the invention would be useful in promoting survival or functioning in a particular cell type.

In certain circumstances, it may be desirable to modulate or decrease the amount of the subject colon or colorectal protein expressed. Thus, in another aspect of the present invention, anti-sense oligonucleotides can be made specific to genes disclosed herein and a method utilized for diminishing the level of expression a protein according to the invention by a cell comprising administering one or more gene anti-sense oligonucleotides. By gene specific anti-sense oligonucleotides reference is made to oligonucleotides that have a nucleotide sequence that interacts through base pairing with a specific complementary nucleic acid sequence involved in the expression of a gene according to the invention that the expression of the gene is reduced. Nucleic acids involved in the expression of the subject gene include genomic DNA and mRNA that encode a colon or colorectal gene disclosed herein. This genomic DNA molecule can comprise regulatory regions of the gene, or the coding sequence for mature gene encoded by the gene.

The term complementary to a nucleotide sequence in the context of antisense oligonucleotides and methods therefor means sufficiently complementary to such a sequence as to allow hybridization to that sequence in a cell, i.e., under physiological conditions. The antisense oligonucleotides can comprise a sequence containing from about 8 to about 100 nucleotides, including antisense oligonucleotides that comprise from about 15 to about 30 nucleotides. The antisense oligonucleotides can also contain a variety of modifications that confer resistance to nucleolytic degradation such as, for example, modified internucleoside linages [Uhlmann and Peyman, Chemical Reviews 90:543-548 (1990); Schneider and Banner, Tetrahedron Lett. 31:335, (1990) which are incorporated by reference], modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Representative modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Representative moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Select of optimal antisense molecules for particular targets typically involves routine screening of a number of candidate molecules. An antisense molecule can be targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, a human or non-human primate protein according to the invention can also be linked or conjugated with agents that provide desirable pharmaceutical or pharmacodynamic properties. For example, the protein can be coupled to any substance known in the art to promote penetration or transport across the blood-brain barrier such as an antibody to the transferrin receptor, and administered by intravenous injection (see, for example, Friden et al., Science 259:373-377 (1993) which is incorporated by reference). Furthermore, the subject protein can be stably linked to a polymer such as polyethylene glycol to obtain desirable properties of solubility, stability, half-life and other pharmaceutically advantageous properties. [See, for example, Davis et al., Enzyme Eng. 4:169-73 (1978); Buruham, Am. J. Hosp. Pharm. 51:210-218 (1994) which are incorporated by reference].

The compositions are usually employed in the form of pharmaceutical preparations, which are made in a manner well known in the pharmaceutical art. See, e.g. Remington Pharmaceutical Science, 18th Ed., Merck Publishing Co. Eastern Pa., (1990). Physiological saline solutions can be used, as well as other pharmaceutically acceptable carriers such as physiological concentrations of other non-toxic salts, five percent aqueous glucose solution, sterile water and the like. Compositions of the invention can also include a suitable buffer. Optionally, such solutions can be lyophilized and stored in a sterile ampoule ready for reconstitution by the addition of sterile water for ready injection. The primary solvent can be aqueous or alternatively non-aqueous. The subject human or primate protein, fragment or variant thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier can contain still other pharmaceutically-acceptable excipients for modifying or maintaining release or absorption or penetration across the blood-brain barrier. Excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing a protein according to the invention or variant or fragment thereof are to be administered orally. Protein formulations can be encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions can be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose also depends on the particular route of administration selected. Further refinement of the calculations necessary to determine the appropriate dosage for treatment is routinely made by those of ordinary skill in the art. Following a review of the present disclosure, an effective dosage can be determined without undue experimentation. Exact dosages are determined in conjunction with standard dose-response studies. The amount of the composition actually administered can be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment, a protein of the present invention is therapeutically administered by implanting into patients vectors or cells capable of producing a biologically-active form of the protein or a precursor of the protein, i.e., a molecule that can be readily converted to a biological-active form of the by the body. For example, cells that secrete the protein can be encapsulated into semipermeable membranes for implantation into a patient. The cells can be cells that normally express the protein or a precursor thereof or the cells can be transformed to express the protein or a precursor thereof. For human subjects, a human protein can be used, or a non-human primate protein homolog of a human protein can be used.

In a number of circumstances it would be desirable to determine the levels of protein or corresponding mRNA encoding a protein according to the invention in a patient. The identification of the subject genes which are specifically expressed by colon or colorectal tumors suggests these proteins are expressed at different levels during some diseases, e.g., cancers, provides the basis for the conclusion that the presence of these proteins serves a normal physiological function related to cell growth and survival. Endogenously produced human colon or colorectal antigen according to the invention may also play a role in certain disease conditions.

The term “detection” as used herein in the context of detecting the presence of a cancer gene according to the invention in a patient is intended to include the determining of the amount of protein according to the invention or the ability to express an amount of this protein in a patient, the estimation of prognosis in terms of probable outcome of a disease and prospect for recovery, the monitoring of these protein levels over a period of time as a measure of status of the condition, and the monitoring of colon or colorectal protein according to the invention for determining an effective therapeutic regimen for the patient, e.g. one with colon cancer.

To detect the presence of a gene according to the invention in a patient, a sample is obtained from the patient. The sample can be a tissue biopsy sample or a sample of blood, plasma, serum, CSF or the like. It has been found that the subject genes are expressed at high levels in some cancers, e.g., colon or colorectal cancers. Samples for detecting protein can be taken from these tissue. When assessing peripheral levels of protein, a sample of blood, plasma or serum can be used. When assessing the levels of protein in the central nervous system, samples can be obtained from cerebrospinal fluid or neural tissue.

In some instances, it is desirable to determine whether a gene according to the invention is intact in the patient or in a tissue or cell line within the patient. By an intact gene, it is meant that there are no alterations in the gene such as point mutations, deletions, insertions, chromosomal breakage, chromosomal rearrangements and the like wherein such alteration might alter the production of gene or alter its biological activity, stability or the like to lead to disease processes. Thus, in one embodiment of the present invention a method is provided for detecting and characterizing any alterations in the gene. The method comprises providing an oligonucleotide that contains the gene corresponding cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize specifically to the gene. A nucleic acid of the invention can be isolated, chemically synthesized, of recombinantly produced (e.g., using in vitro DNA replication, reverse transcription, or transcription).

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact gene according to the invention or a gene abnormality.

Hybridization to a gene according to the invention would involve denaturing the chromosomal DNA to obtain a single-stranded DNA; contacting the single-stranded DNA with a gene probe associated with the gene sequence; and identifying the hybridized DNA-probe to detect chromosomal DNA containing at least a portion of a human gene according to the invention.

The term “probe” as used herein refers to a structure comprised of a polynucleotide that forms a hybrid structure with a target sequence, due to complementarity of probe sequence with a sequence in the target region. Oligomers suitable for use as probes typically contain at least about 8-12 contiguous nucleotides which are complementary to the targeted sequence, for example 20 nucleotides.

Probes of the present invention can be DNA or RNA oligonucleotides and can be made by any method known in the art such as, for example, excision, transcription or chemical synthesis. Probes can be labeled with any detectable label known in the art such as, for example, radioactive or fluorescent labels or enzymatic marker. Labeling of the probe can be accomplished by any method known in the art such as by PCR, random priming, end labeling, nick translation or the like. Methods that do not employ a labeled probe can also be used to determine the hybridization. Representative techniques include Southern blotting, fluorescence in situ hybridization, and single-strand conformation polymorphism with PCR amplification.

Hybridization is typically carried out at about 25°-45° C., or at about 32°-40° C., or at about 37°-38° C. Hybridization can proceed for about 0.25 hour to about 96 hours, or from about 1 (one) hour to about 72 hours, or from about 4 hours to about 24 hours.

Gene abnormalities can also be detected by using the PCR method and primers that flank or lie within the particular gene. The PCR method is well known in the art. Briefly, this method is performed using two oligonucleotide primers which are capable of hybridizing to the nucleic acid sequences flanking a target sequence that lies within gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising a gene of the invention or a fragment thereof is then directly sequenced and analyzed by comparison of the sequence with the sequences disclosed herein to identify alterations which might change activity or expression levels or the like.

In another embodiment, a method for detecting protein a colon according to the invention is provided based upon an analysis of tissue expressing the gene. Certain tissues such as breast, lung, colon and others can be analyzed. The method comprises hybridizing a polynucleotide to mRNA from a sample of tissue that normally expresses the gene. The sample is obtained from a patient suspected of having an abnormality in the gene.

To detect the presence of mRNA encoding protein a colon or colorectal protein according to the invention is obtained from a patient. The sample can be from blood or from a tissue biopsy sample. The sample can be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding a colon or colorectal protein according to the invention or a derivative of the cDNA as a probe, high stringency conditions can be used in order to prevent false positives, that is the hybridization and apparent detection of the gene nucleotide sequences when in fact an intact and functioning gene is not present. When using sequences derived from the gene or cDNA, less stringent conditions could be used, however, are less preferred because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding the protein, the technique of reverse transcription/polymerization chain reaction (RT/PCR) can be used to amplify cDNA transcribed from mRNA encoding the protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and specific primers. [Belyavsky et al., Nucl. Acid Res. 17:2919-2932 (1989); Krug and Berger, Methods in Enzymology, 152:316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of a colon or colorectal protein in a sample obtained from a patient. Any method known in the art for detecting proteins can be used. Representative methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. For example, binder-ligand immunoassays can be used, which involve reacting antibodies with an epitope or epitopes of a colon protein of the invention and competitively displacing a labeled protein or derivative thereof.

As used herein, a derivative of a protein according to the invention is intended to include a polypeptide in which certain amino acids have been deleted or replaced or changed to modified or unusual amino acids wherein the derivative is biologically equivalent to the gene and wherein the polypeptide derivative cross-reacts with antibodies raised against the protein. By cross-reaction it is meant that an antibody reacts with an antigen other than the one that induced its formation.

Numerous competitive and non-competitive protein-binding immunoassays are well known in the art. Antibodies employed in such assays can be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the subject non-human primate or human proteins or according to the invention an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, typically a rabbit, hamster or a mouse.

Oligopeptides can be selected as candidates for the production of an antibody to the subject colon or colorectal protein based upon the oligopeptides lying in hydrophilic regions, which are thus likely to be exposed in the mature protein.

Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for a protein according to the invention. The phrase “humanized antibody” refers to an antibody derived from a non-human antibody, typically a mouse monoclonal antibody. Alternatively, a humanized antibody can be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments generally human constant and mouse variable regions.

Because humanized antibodies are far less immunogenic in humans than the parental mouse monoclonal antibodies, they can be used for the treatment of humans with far less risk of anaphylaxis. Thus, these antibodies are useful in therapeutic applications that involve in vivo administration to a human such as, e.g., use as radiation sensitizers for the treatment of neoplastic disease or use in methods to reduce the side effects of, e.g., cancer therapy.

Humanized antibodies can be prepared using a variety of techniques including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies include both “humanized” and “veneered” antibodies. These methods are disclosed in, e.g., Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3): 169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin-binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject-humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Humanized antibodies can be further derivatized to facilitate uptake and clearance, e.g, via Ashwell receptors. See, e.g., U.S. Pat. Nos. 5,530,101 and 5,585,089 which patents are incorporated herein by reference.

Humanized antibodies to proteins according to the invention can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, proteins and variants thereof according to the invention are used to immunize a transgenic animal as described above. Monoclonal antibodies are made using methods known in the art, and the specificity of the antibodies is tested using isolated colon or colorectal proteins according to the invention.

Methods for preparation of the human or primate protein according to the invention or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system [E.I. du Pont de Nemours Company, Wilmington, Del.) (Caprino and Han, J. Org. Chem. 37:3404 (1972) which is incorporated by reference].

Polyclonal antibodies can be prepared by immunizing rabbits or other animals by injecting antigen followed by subsequent boosts at appropriate intervals. The animals are bled and sera assayed against purified protein usually by ELISA or by bioassay based upon the ability to block the action of a gene according to the invention. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256:495-497 (1975); Gulfre and Milstein, Methods in Enzymology: Immunochemical Techniques 73:1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the a protein according to the invention by treatment of a patient with antibodies to specific tumor antigen according to the invention.

Specific antibodies, either polyclonal or monoclonal, to the protein can be produced by any suitable method known in the art as discussed above. For example, murine or human monoclonal antibodies can be produced by hybridoma technology or, alternatively, the tumor protein, or an immunologically active fragment thereof, or an anti-idiotypic antibody, or fragment thereof can be administered to an animal to elicit the production of antibodies capable of recognizing and binding to the tumor protein. Antibodies can be of any class or subclass, e.g., IgG, IgA, IgM, IgD, and IgE or in the case of avian species, IgY, and subclasses thereof.

The availability of isolated human or primate protein according to the invention allows for the identification of small molecules and low molecular weight compounds that inhibit the binding of the protein to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds can be identified via the incorporation of radioactivity or through optical assays that rely on absorbance, fluorescence or luminescence as read-outs. [Gonzalez, J. E. et al., Curr. Opin. Biotech. 9:624-631 (1998)].

Model systems are available that can be adapted for use in high throughput screening for compounds that inhibit the interaction of a protein with its ligand, for example by competing with the protein for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

The therapeutic gene polynucleotides and polypeptides of the present invention can be utilized in gene delivery vehicles. The gene delivery vehicle can be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Recombinant retroviruses useful in accordance with the present invention include those described in WO 91/02805.

Packaging cell lines suitable for use with the above-described retroviral vector constructs can be readily prepared (see PCT publications WO 95/30763 and WO 92/05266), and used to create producer cell lines (also termed vector cell lines) for the production of recombinant vector particles. For example, packaging cell lines can be prepared from human (such as HT1080 cells) or mink parent cell lines, thereby allowing production of recombinant retroviruses that can survive inactivation in human serum.

The present invention also employs alphavirus-based vectors that can function as gene delivery vehicles. Vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63: 3822-3828 (1989); Mendelson et al., Virol. 166: 154-165 (1988); and Flotte et al., P.N.A.S. 90: 10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisleret al., P.N.A.S. 90: 11498-11502 (1993); Guzman et al., Circulation 88: 2838-2848 (1993); Guzman et al., Cir. Res. 73: 1202-1207 (1993); Zabner et al., Cell 75: 207-216 (1993); Li et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to kill adenovirus as described in Curiel, Hum. Gene Ther. 3: 147-154 (1992) can be employed.

Other gene delivery vehicles and methods can be employed, including polycationic condensed DNA linked or unlinked to kill adenovirus alone, for example Curiel, Hum. Gene Ther. 3: 147-154 (1992); ligand-linked DNA, for example see Wu, J. Biol. Chem. 264: 16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA can also be administered directly to a subject. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Identification of CICO1-CICO3 Genes

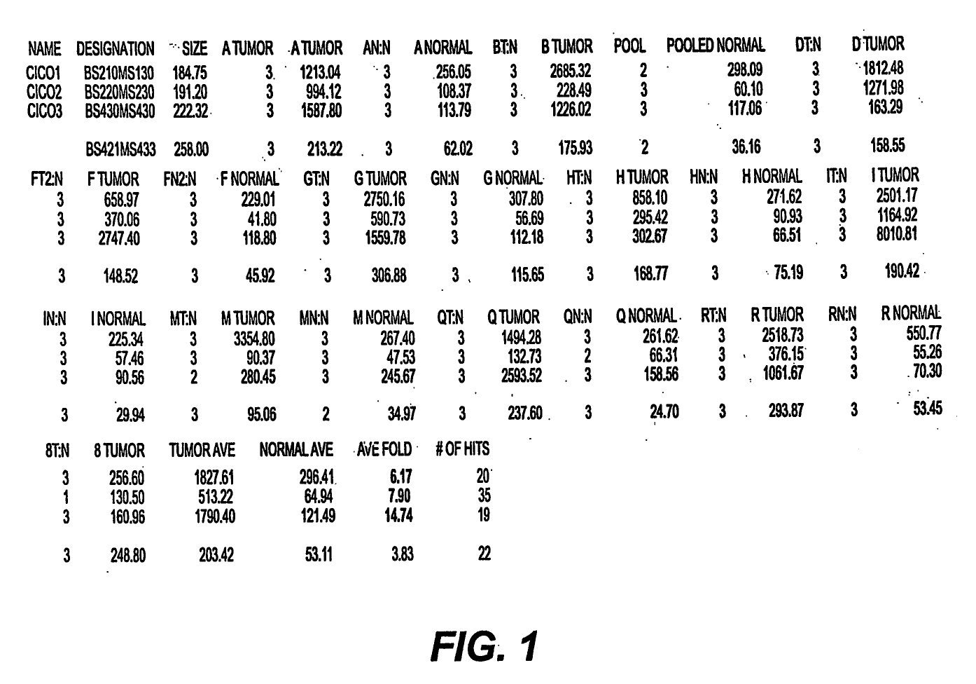

Through a collaboration with Analytical Pathology Medical Group (at Grossmont Hospital), IDEC obtained pairs of snap frozen normal and malignant colon tissue removed during surgery. RNA was extracted from 10 pairs of those samples and submitted for GENETAG® analysis at Celera/Applied Bio Systems (ABI). In brief, the RNA was reverse transcribed into cDNA, digested with a restriction enzyme, and linkers were ligated to the cDNA library. The library was amplified using the linker sequences as a primer with an additional nucleotide (A, T, G, or C) (+1 PCR) to generate 16 libraries. The libraries were further amplified using the linker sequences as primers with an additional two nucleotides (+2 PCR) to generate 256 libraries. Fluorescently labeled products from these +2 PCR reactions were separated by capillary electrophoresis and the amplified sequences were quantitated. The expression profile obtained from malignant colon RNA was compared to that obtained using RNA from the normal colon. Several sequences were identified to be at least five-fold overexpressed in three of three tumors. The expression results are summarized in FIG. 1. Overexpressed sequences were purified and amplified by PCR using the linkers with three additional nucleotides (+3 PCR). The +3 peaks were purified and sequenced. These sequences are set forth below:

CICO1 (Celera IDEC Colon Overexpressed 1) (bs213 ms134-185)

Using 185 bases of +3 PCR sequence from GENETAG® bs213 ms134, human tentative human consensus sequence (THC) 684921 was identified from the BLAST database.

bs213ms143-185 Nucleotide Sequence

(SEQ ID NO:1)

GATCCAGGAGAGGAAGGAGTTTCAGAAGGCAGGAGCTGGTCCTCTATGTC
ATGAAATGTAGAGGGTGAGGCCAAGGAGGACCTGAGAGAAGGTAATTAGA
TTTGGTGTTTACAGGCTGGTCCCTGTGGCCAGCCACCCCACCCACTTTA

THC 684921 Nucleotide Sequence

(SEQ ID NO:2)

TGAGGAAACTGTGGCTTAGAGGAAAAGGTCATTAGTTCATTTTGGGATTT
GTTGATTTTCAGATGTTTGAGATGTTGAGGATGGATTGTCCAGCAGGCTA
TTAAGATGTGGTGAAGGCTAGAAATGTTGATTTAGGAGGTATTGCCTTCG
AGAAGATAAAGGAGGAGAAGAGGAGAGCATCATGCAAGCTAGAGAAGAGA
AAGAAGAAAAGTATTCTGGGGAATGTCTCCTTTGGGAGCAGAAAGAAGAC
TCTGACGGAGCAGCCATCCAGGAAGTGGAATGAGATCCAGGAGAGGAAGG
AGTTTCAGAAGGCAGGAGCTGGTCCTCTATGTCATGAAATGTAGAGGGTG
AGGCCAAGGAGGACCTGAGAGAAGGTAATTAGATTTGGTGTTTACAGGCT
GGTCCCTGTGGCCAGCCACCCCACCCACTTTAAAATATTTACTCTACAAA
TGTTAATGTGTGAAGAGTTGCATGCCAGAATATTTATGGCATCAGTGTTG
GTGGATACAGAACATTGGGAAACAACCCATTAATAGCAGAATGGTAAATC
TGGCCAGTGAATAGTATAGCTTTTTAAAAGGAGGCTGATGTCTGAATTCA
CTTTCAAAGTTGTTCACAATGTATTGCTAAAATACAAAAATGTTGCAGAA
CCATATGTATGAGAGAAACCCCTTTTTCT

CICO 2 (bs222 ms233-191)

191 bases of the +3 PCR sequence from GENETAG® bs222 ms233-191 overlapped with the 3′UTR of four different hypothetical proteins in the BLAST database.

bs222ms233-191 Nucleotide Sequence

(SEQ ID NO:3)

gatccccatggtatgcttgaatctgctccctgaacttcctgccagtgcct
ccccgtaccccaaaacaatgtcaccatggttaccacctacccagaagact
gttccctcctcccaagacccttgtctgcagtggtgctcctgcaggctgcc
cgtta

chr1_70_2399.c mRNA Sequence
(coding sequence in CAPITALS, no ATG at start)

(SEQ ID NO:4)

AGTGTGGTGATGGTTGTCTTCGACAATGAGAAGGTCCCAGTAGAGCAGCT
GCGCTTCTGGAAGCACTGGCATTCCCGGCAACCCACTGCCAAGCAGCGGG
TCATTGACGTGGCTGACTGCAAAGAAAACTTCAACACTGTGGAGCACATT
GAGGAGGTGGCCTATAATGCACTGTCCTTTGTGTGGAACGTGAATGAAGA
GGCCAAGGTGTTCATCGGCGTAAACTGTCTGAGCACAGACTTTTCCTCAC
AAAAGGGGGTGAAGGGTGTCCCCCTGAACCTGCAGATTGACACCTATGAC
TGTGGCTTGGGCACTGAGCGCCTGGTACACCGTGCTGTCTGCCAGATCAA
GATCTTCTGTGACAAGGGAGCTGAGAGGAAGATGCGCGATGACGAGCGGA
AGCAGTTCCGGAGGAAGGTCAAGTGCCCTGACTCCAGCAACAGTGGCGTC
AAGGGCTGCCTGCTGTCGGGCTTCAGGGGCAATGAGACGACCTACCTTCG
GCCAGAGACTGACCTGGAGACGCCACCCGTGCTGTTCATCCCCAATGTGC
ACTTCTCCAGCCTGCAGCGGTCTGGAGGGGCAGCCCCCTCGGCAGGACCC
AGCAGCTCCAACAGGCTGCCTCTGAAGCGTACCTGCTCGCCCTTCACTGA
GGAGTTTGAGCCTCTGCCCTCCAAGCAGGCCAAGGAAGGCGACCTTCAGA
GAGTTCTGCTGTATGTGCGGAGGGAGACTGAGGAGGTGTTTGACGCGCTC
ATGTTGAAGACCCCAGACCTGAAGGGGCTGAGGAATGCGATCTCTGAGAA
GTATGGGTTCCCTGAAGAGAACATTTACAAAGTCTACAAGAAATGCAAGC
GAGGAATCTTAGTCAACATGGACAACAACATCATTCAGCATTACAGCAAC
CACGTCGCCTTCCTGCTGGACATGGGGGAGCTGGACGGCAAAATTCAGAT
CATCCTTAAGGAGCTGTAAggcctctcgagcatccaaaccctcacgacct
gcaaggggccagcagggacgtggccccacgccacacacaacctctccaca
tgcctcagcgctgttacttgaatgccttccctgagggaagaggcccttga
gtcacagacccacagacgtcagggccagggagagacctagggggtcccct
ggcctggatccccatggtatgcttgaatctgctccctgaacttcctgcca
gtgcctccccgtaccccaaaacaatgtcaccatggttaccacctacccag
aagactgttccctcctcccaagacccttgtctgcagtggtgctcctgcag
gctgcccgttaagatggtggcggcacacgctccctcccgcagcaccacgc
cagctggtgcggcccccactctctgtcttccttcaacttcagacaaagga
tttctcaacctttggtcagttaacttgaaaactcttgattttcagtgcaa
atgacttttaaaagacactatattggagtctctttctcagacttcctcag
cgcaggatgtaaatagcactaacgatcgactggaacaaagtgaccgctgt
gtaaaactactgccttgccactcactgttgtatacatttcttatttacga
ttttcatttgttatatatatatataaatatactgtatatatatgcaacat
tttatatttttcatggatatgtttttatcatttcaaaaaatgtgtatttc
acatttcttggactttttttagctgttattcagtgatgcattttgtatac
tcacgtggtatttagtaataaaaatctatctatgtattacgtcac

chr1_70_2399.c Amino Acid Sequence

(SEQ ID NO:5)

SVVMVVFDNEKVPVEQLRFWKHWHSRQPTAKQRVIDVADCKENFNTVEHI
EEVAYNALSFVWNVNEEAKVFIGVNCLSTDFSSQKGVKGVPLNLQIDTYD
CGLGTERLVHRAVCQIKIFCDKGAERKMRDDERKQFRRKVKCPDSSNSGV
KGCLLSGFRGNETTYLRPETDLETPPVLFIPNVHFSSLQRSGGAAPSAGP
SSSNRLPLKRTCSPFTEEFEPLPSKQAKEGDLQRVLLYVRRETEEVFDAL
MLKTPDLKGLRNAISEKYGFPEENIYKVYKKCKRGILVNMDNNIIQHYSN
HVAFLLDMGELDGKIQIILKEL

chr1_70_2399.f mRNA Sequence
(coding sequence in CAPITALS, no ATG at start)

(SEQ ID NO:6)

aagttgccccacctctctgagcattggcttccccatctgtgaaagaggag
tgctgatgtttgccttctaggggcctagtgaggcttaagggtgagcagca
ggcacacagaaagctagaaatacaggatcactgtgggacggtggggctgg
ccacctgggcaggccacttacccagcggccccctctgtctccaggtgttc
atcggcgtaaactgtctgagcacagacttttcctcacaaaagggggtgaa
gggtgtccccctgaacctgcagattgacacctatgactgtggcttgggca
ctgagcgcctggtacaccgtgctgtctgccagatcaagatcttctgtgac
aagggagctgagaggaagatgcgcgatgacgagcggaagcagttccggag
gaaggtcaagtgccctgactccagcaacagtggcgtcaagggctgcctgc
tgtcgggcttcaggggcaatgagacgacctaccttcggccagagactgac
ctggagacgccacccgtgctgttcatccccaatgtgcacttctccagcct
gcagcggtctggaggggcagccccctcggcaggacccagcagctccaaca
ggctgcctctgaagcgtacctgctcgcccttcactgaggagtttgagcct
ctgccctccaagcaggccaaggaaggcgaccttcagagagttctgctgta
tgtgcggagggagactgaggaggtgtttgacgcgctcatgttgaagaccc
cagacctgaaggggctgaggaatgcgatctctgagaagtatgggttccct
gaaGAGAACATTTACAAAGTCTACAAGAAATGCAAGCGAGGAATCTTAGT
CAACATGGACAACAACATCATTCAGCATTACAGCAACCACGTCGCCTTCC
TGCTGGACATGGGGGAGCTGGACGGCAAAATTCAGATCATCCTTAAGGAG
CTGTAAggcctctcgagcatccaaaccctcacgacctgcaaggggccagc
agggacgtggccccacgccacacacaacctctccacatgcctcagcgctg
ttacttgaatgccttccctgagggaagaggcccttgagtcacagacccac
agacgtcagggccagggagagacctagggggtcccctggcctggatcccc
atggtatgcttgaatctgctccctgaacttcctgccagtgcctccccgta
ccccaaaacaatgtcaccatggttaccacctacccagaagactgttccct
cctcccaagacccttgtctgcagtggtgctcctgcaggctgcccgttaag
atggtggcggcacacgctccctcccgcagcaccacgccagctggtgcggc
ccccactctctgtcttccttcaacttcagacaaaggatttctcaaccttt
ggtcagttaacttgaaaactcttgattttcagtgcaaatgacttttaaaa
gacactatattggagtctctttctcagacttcctcagcgcaggatgtaaa
tagcactaacgatcgactggaacaaagtgaccgctgtgtaaaactactgc
cttgccactcactgttgtatacatttcttatttacgattttcatttgtta
tatatatatataaatatactgtatatatatgcaacattttatatttttca
tggatatgtttttatcatttcaaaaaatgtgtatttcacatttcttggac
tttttttagctgttattcagtgatgcattttgtatactcacgtggtattt
agtaataaaaatctatctatgtattacgtcac

chr1_70_2399.f Amino Acid Sequence

(SEQ ID NO:7)

MRDDERKQFRRKVKCPDSSNSGVKGCLLSGFRGNETTYLRPETDLETPPV
LFIFNVHFSSLQRSGGAAPSAGPSSSNRLPLKRTCSPFTEEFEPLPSKQA
KEGDLQRVLLYVRRETEEVFDALMLKTPDLKGLRNAISEKYGFPEENIYK
VYKKCKRGILVNMDNNIIQHYSNHVAFLLDMGELDGKIQIILKEL

C1000572 mRNA Sequence (coding)

(SEQ ID NO:8)

ATGAAAAGGTCTGTGCGGCTGCTAAAGAACGACCCAGTCAACTTGCAGAA
ATTCTCTTACACTAGTGAGGATGAGGCCTGGAAGACGTACCTAGAAAACC
CGTTGACAGCTGCCACAAAGGCCATGATGAGAGTCAATGGAGATGATGAG
AGTGTTGCGGCCTTGAGCTTCCTCTATGATTACTACATGTCGATGCTCTT
CCCAGATATCCTGAAAACCTCCCCGGAACCCCCATGTCCAGAGGACTACC
CCAGCCTCAAAAGTGACTTTGAATACACCCTGGGCTCCCCCAAAGCCATC
CACATCAAGTCAGGCGAGTCACCCATGGCCTACCTCAACAAAGGCCAGTT
CTACCCCGTCACCCTGCGGACCCCAGCAGGTGGCAAAGGCCTTGCCTTGT
CCTCCAACAAAGTCAAGAGTGTGGTGATGGTTGTCTTCGACAATGAGAAG
GTCCCAGTAGAGCAGCTGCGCTTCTGGAAGCACTGGCATTCCCGGCAACC
CACTGCCAAGCAGCGGGTCATTGACGTGGCTGACTGCAAAGAAAACTTCA
ACACTGTGGAGCACATTGAGGAGGTGGCCTATAATGCACTGTCCTTTGTG
TGGAACGTGAATGAAGAGGCCAAGGTGTTCATCGGCGTAAACTGTCTGAG
CACAGACTTTTCCTCACAAAAGGGGGTGAAGGGTGTCCCCCTGAACCTGC
AGATTGACACCTATGACTGTGGCTTGGGCACTGAGCGCCTGGTACACCGT
GCTGTCTGCCAGATCAAGATCTTCTGTGACAAGGGAGCTGAGAGGAAGAT
GCGCGATGACGAGCGGAAGCAGTTCCGGAGGAAGGTCAAGTGCCCTGACT
CCAGCAACAGTGGCGTCAAGGGCTGCCTGCTGTCGGGCTTCAGGGGCAAT
GAGACGACCTACCTTCGGCCAGAGACTGACCTGGAGACGCCACCCGTGCT
GTTCATCCCCAATGTGCACTTCTCCAGCCTGCAGCGGTCTGGAGGGAGCC
TCCAGCAGCCAGGGGCTCCTCTCATTTTCCTGCGTGTGATGGAAAATGTC
TTTTTCACTTCATTGCAGGCAGCCCCCTCGGCAGGACCCAGCAGCTCCAA
CAGGCTGCCTCTGAAGCGTACCTGCTCGCCCTTCACTGAGGAGTTTGAGC
CTCTGCCCTCCAAGCAGGCCAAGGAAGGCGACCTTCAGAGAGTTCTGCTG
TATGTGCGGAGGGAGACTGAGGAGGTGTTTGACGCGCTCATGTTGAAGAC
CCCAGACCTGAAGGGGCTGAGGAATGCGATCTCTGAGAAGTATGGGTTCC
CTGAAGAGAACATTTACAAAGTCTACAAGAAATGCAAGCGAGGAATCTTA
GTCAACATGGACAACAACATCATTCAGCATTACAGCAACCACGTCGCCTT
CCTGCTGGACATGGGGGAGCTGGACGGCAAAATTCAGATCATCCTTAAGG
AGCTGTAA

C1000572 Amino Acid Sequence

(SEQ ID NO:9)

MKRSVRLLKNDPVNLQKFSYTSEDEAWKTYLENPLTAATKAMMRVNGDDE
SVAALSFLYDYYMSMLFPDILKTSPEPPCPEDYPSLKSDFEYTLGSPKAI
HIKSGESPMAYLNKGQFYPVTLRTPAGGKGLALSSNKVKSVVMVVFDNEK
VPVEQLRFWKHWHSRQPTAKQRVIDVADCKENFNTVEHIEEVAYNALSFV
WNVNEEAKVFIGVNCLSTDFSSQKGVKGVPLNLQIDTYDCGLGTERLVHR
AVCQIKIFCDKGAERKMRDDERKQFRRKVKCPDSSNSGVKGCLLSGFRGN
ETTYLRPETDLETPPVLFIPNVHFSSLQRSGGSLQQPGAPLIFLRVMENV
FFTSLQAAPSAGPSSSNRLPLKRTCSPFTEEFEPLPSKQAKEGDLQRVLL
YVRRETEEVFDALMLKTPDLKGLRNAISEKYGFPEENIYKVYKKCKRGIL
VNMDNNIIQHYSNHVAFLLDMGELDGKIQIILKEL

ctgChr_1ctg20.176 mRNA Sequence (coding)

(SEQ ID NO:10)

ATGGAGGCAGGGGAGAAAAGCGCTCTGGGTGCCTGGAGCCCGCAGCCCTG
GGCAGCCCCGGGCTACCGCAGGGCGCAAGGGATCCTGGGCTGCGGCCGAG
GGCGCCGGAAGTCGCCGCCGACCGCCTGGGTCTCGCAGGAAAACAGCCGG
CGCCCGCGAGCTGCCCAGCGTCGGGTTTTCCTGAAGAGCCCAGCTCCTCA
CACCTTGGGGCCTGGTGGGATGGGAGACACTGTCCTGGATGAAGCCGCTG
GGAGAGCTGCCGCCTCCTGTATGCTGAGGTCTGTGCGGCTGCTAAAGAAC
GACCCAGTCAACTTGCAGAAATTCTCTTACACTAGTGAGGATGAGGCCTG
GAAGACGTACCTAGAAAACCCGTTGACAGCTGCCACAAAGGCCATGATGA
GAGTCAATGGAGATGATGAGAGTGTTGCGGCCTTGAGCTTCCTCTATGAT
TACTACATGGGTCCCAAGGAGAAGCGGATATTGTCCTCCAGCACTGGGGG
CAGGAATGACCAAGGAAAGAGGTACTACCATGGCATGGAATATGAGACGG
ACCTCACTCCCCTTGAAAGCCCCACACACCTCATGAAATTCCTGACAGAG
AACGTGTCTGGAACCCCAGAGTACCCAGATTTGCTCAAGAAGAATAACCT
GATGAGCTTGGAGGGGGCCTTGCCCACCCCTGGCAAGGCAGCTCCCCTCC
CTGCAGGCCCCAGCAAGCTGGAGGCCGGCTCTGTGGACAGCTACCTGTTA
CCCACCACTGATATGTATGATAATGGCTCCCTCAACTCCTTGTTTGAGAG
CATTCATGGGGTGCCGCCCACACAGCGCTGGCAGCCAGACAGCACCTTCA
AAGATGACCCACAGGAGTCGATGCTCTTCCCAGATATCCTGAAAACCTCC
CCGGAACCCCCATGTCCAGAGGACTACCCCAGCCTCAAAAGTGACTTTGA
ATACACCCTGGGCTCCCCCAAAGCCATCCACATCAAGTCAGGCGAGTCAC
CCATGGCCTACCTCAACAAAGGCCAGTTCTACCCCGTCACCCTGCGGACC
CCAGCAGGTGGCAAAGGCCTTGCCTTGTCCTCCAACAAAGTCAAGAGTGT
GGTGATGGTTGTCTTCGACAATGAGAAGGTCCCAGTAGAGCAGCTGCGCT
TCTGGAAGCACTGGCATTCCCGGCAACCCACTGCCAAGCAGCGGGTCATT
GACGTGGCTGACTGCAAAGAAAACTTCAACACTGTGGAGCACATTGAGGA
GGTGGCCTATAATGCACTGTCCTTTGTGTGGAACGTGAATGAGAAGGCCA
AGGTGTTCATCGGCGTAAACTGTCTGAGCACAGACTTTTCCTCACAAAAG
GGGGTGAAGGGTGTCCCCCTGAACCTGCAGATTGACACCTATGACTGTGG
CTTGGGCACTGAGCGCCTGGTACACCGTGCTGTCTGCCAGATCAAGATCT
TCTGTGACAAGGGAGCTGAGAGGAAGATGCGCGATGACGAGCGGAAGCAG
TTCCGGAGGAAGGTCAAGTGCCCTGACTCCAGCAACAGTGGCGTCAAGGG
CTGCCTGCTGTCGGGCTTCAGGGGCAATGAGACGACCTACCTTCGGCCAG
AGACTGACCTGGAGACGCCACCCGTGCTGTTCATCCCCAATGTGCACTTC
TCCAGCCTGCAGCGGTCTGGAGGGCTCCAACTGCCTAGTTACCGGCCGCA
GGACCATCTGCAATTCCCAGCCCTTCTGGGCATGCTGGGGCCCAGGCTGC
CTCTGAAGCGTACCTGCTCGCCCTTCACTGAGGAGTTTGAGCCTCTGCCC
TCCAAGCAGGCCAAGGAAGGCGACCTTCAGAGAGTTCTGCTGTATGTGCG
GAGGGAGACTGAGGAGGTGTTTGACGCGCTCATGTTGAAGACCCCAGACC
TGAAGGGGCTGAGGAATGCGATCTCTGAGAAGTATGGGTTCCCTGAAGAG
AACATTTACAAAGTCTACAAGAAATGCAAGCGAGGAATCTTAGTCAACAT
GGACAACAACATCATTCAGCATTACAGCAACCACGTCGCCTTCCTGCTGG
ACATGGGGGAGCTGGACGGCAAATTCAGATCATCCTTAAGGAGCTGTAA

ctgChr_1ctg20.176 Amino Acid Sequence

(SEQ ID NO:11)

MEAGEKSALGAWSPQPWAAPGYRRAQGILGCGRGRRKSPPTAWVSQENSR
RPRAAQRRVFLKSPAPHTLGPGGMGDTVLDEAAGRAAASCMLRSVRLLKN
DPVNLQKFSYTSEDEAWKTYLENPLTAATKAMMRVNGDDESVAALSFLYD
YYMGPKEKRILSSSTGGRNDQGKRYYHGMEYETDLTPLESPTHLMKFLTE
NVSGTPEYPDLLKKNNLMSLEGALPTPGKAAPLPAGPSKLEAGSVDSYLL
PTTDMYDNGSLNSLFESIHGVPPTQRWQPDSTFKDDPQESMLFPDILKTS
PEPPCPEDYPSLKSDFEYTLGSPKAIHIKSGESPMAYLNKGQFYPVTLRT
PAGGKGLALSSNKVKSVVMVVFDNEKVPVEQLRFWKHWHSRQPTAKQRVI
DVADCKENFNTVEHIEEVAYNALSFVWNVNEEAKVFIGVNCLSTDFSSQK
GVKGVPLNLQIDTYDCGLGTERLVHRAVCQIKIFCDKGAERKMRDDERKQ
FRRKVKCPDSSNSGVKGCLLSGFRGNETTYLRPETDLETPPVLFIPNVHF
SSLQRSGGLQLPSYRPQDHLQFPALLGMLGPRLPLKRTCSPFTEEFEPLP
SKQAKEGDLQRVLLYVRRETEEVFDALMLKTPDLKGLRNAISEKYGFPEE

CICO3 (bs432 ms434-222)

The 222 bases of the +3 PCR sequence from GENETAG® bs432 ms434-222 overlapped with the 3′UTR of two different hypothetical proteins in the BLAST database.

bs432ms434-222 Nucleotide Sequence

(SEQ ID NO:12)

GATCTGCAATCAGAACTATTGAACTTCTCCATTCAGACCGCCACTCACAC
CTATGGGAAAAGGGTAATGTATCATCGGCTTAGCAACAGGGAATACTATT
CGTATGATGGAAAATGGGGACAAAAGGCTTTGGTACATAAAACATTATTC
CTTCCTTGGCCTAAAAACTCATCGCCACCTACATTA

chr19_53_399.c mRNA Sequence

(SEQ ID NO:13)

tctggagcagctgaaaaacaaggaagtgaaacagccaattcctgccttaa
ctaattaacccaccttacgacattccaccattatgacgtgttcctgccct
gccccaactgatcaatcgaccctgtgacattcttctggacaatgagtccc
atcatctctccaccatgcaccttgtgactccctcctctgctgacaacaga
taaccacctttaactgtaactttccacagcctaccccagccctataaagc
tgcccctctcctatctcccttcgctgactctcttttcagactcagcccac
ttgcacccaagtgaattaacagccttgttgctcacacaaagcctgtttag
gtggtcttctatacggacatgcttgacacttggtgccaaaatctgggcca
gggggactccttcgtgagaccggccccctgtcctggccctcattccgtga
agagatccacctgcgacctcgggtcctcagaccagcccaaggaacatctc
accaatttcaaatcggatctcctcggcttagtggctgaagactgatgctg
cccgatcgcctcagaagccccttggaccatcacagatgccgagcttcggg
taactcttacggtggaggattcccagccatatgaagacaccctagctgga
cgatcagtccttgtcaaaagtctgacccctcaaactctacagcctcaatg
gaccagaccctacccggtcatttatagcacaccaactgccgtccatctgc
aggaccctctccattgggttcaccattccagaataaagccatgcccatca
gacagccagcttgatctctcctcttcctcctggaagccacaagattaggc
cgagagccgatcagacaaacaacctacaacccttaagctcctggcagcgc
ccagccaaggccatgcttccttgcaacactccttccaaatggccatccca
gcatgcttccaagcaggcttcatccgttcctctggaccctcatctcttaa
gacctgccgcctataaaaaggattatatcttgagaccctatcctctaaaa
ttttttccacacccaaaacaaaaaatctctgggtcaaaagtctaaaacgc
ttaggctggcaaccatcagatccttgcccatggtgtcctcaagcctactc
tcatgaaatggacaacagtacacgcatatggggccagttccacatatttg
gcaaccagaccagcatccaggacaacacaaagatctgcaatcagaactat
tgaacttctccattcagaccgccactcacacctatgggaaaagggtaatg
tatcatcggcttagcaacagggaatactattcgtatgatggaaaatgggg
acaaaaggctttggtacataaaacattattccttccttggcctaaaaact
catcgccacctacattaaagctaatatgcctgattactgtttttagagaa
cttattttattagggcagttccaagctcaaaaatacgctaactggcacct
tgttagctacataaaaatgcaccctagacccgaaacttactagactcatt
ataaaattttctttaaggtgtccacgcagtccctggtcacacttgaagca
gtccggagaaatatcagccctaccccagtaatccccagaaggaacttaca
cttttttttaatcttttcctacaacttcatattttataaataaaaagaca
aaaatgtcaggcctgtgagctgaagcttagccattgtaacccctgtgacc
tgcacatatccgtccaggtggcctgcaggagccaagaagtctggagcagc
cgaaaaaccacaaagaagtgaaacagccagttcctgccttaactaattaa
cccaccttacgacattccaccattatgacttgtccaccattatgacttgt
tcctgccctgccccaactgatcaatcaaccctgtgacattcttctcctgg
acaatgagtcccatcatctctccaccatgcaccttgtgaccccctcctct
gctgaggataaccacctttaactgtaactttccacgcctacccaagccct
ataaagctgcccctctcctatctcccttcactgactctcttttcggactc
agcccacttgcacccaagtgaattaacagccttgttgctcacacaaagcc
tgattgggtgtcttctatacggacacgcgtgacaggaacctcaacccaaa
ggcagtctgatgaggtgtctaagataaaagtagcggcacaaaggcttttg
taaacagaggcgtttcatgtggttttcctttcctttccttatatgtgaaa
aggtgacagaaaagaaatcttcctaaaagagtc

chr19_53_399.c Amino Acid Sequence

(SEQ ID NO:14)

MGPVPHIWQPDQHPGQHKDLQSELLNFSIQTATHTYGKRVMYHRLSNREY
YSYDGKWGQKALVHKTLFLPWPKNSSPPTLKLICLITVFRELILLGQFQA
QKYANWHLVSYIKMHPRPETY

chr19_53_399.b mRNA Sequence

(SEQ ID NO:15)
tctggagcagctgaaaaacaaggaagtgaaacagccaattcctgccttaa
ctaattaacccaccttacgacattccaccattatgacgtgttcctgccct
gccccaactgatcaatcgaccctgtgacattcttctggacaatgagtccc
atcatctctccaccatgcaccttgtgactccctcctctgctgacaacaga
taaccacctttaactgtaactttccacagcctaccccagccctataaagc
tgcccctctcctatctcccttcgctgactctcttttcagactcagcccac
ttgcacccaagtgaattaacagccttgttgctcacacaaagcctgtttag
gtggtcttctatacggacatgcttgacacttggtgccaaaatctgggcca
gggggactccttcgtgagaccggccccctgtcctggccctcattccgtga
agagatccacctgcgacctcgggtcctcagaccagcccaaggaacatctc
accaatttcaaatcggatctcctcggcttagtggctgaagactgatgctg
cccgatcgcctcagaagccccttggaccatcacagatgccgagcttcggg
taactcttacggtggaggattcccagccatatgaagacaccctagctgga
cgatcagtccttgtcaaaagtctgacccctcaaactctacagcctcaatg
gaccagaccctacccggtcatttatagcacaccaactgccgtccatctgc
aggaccctctccattgggttcaccattccagaataaagccatgcccatca
gacagccagcttgatctctcctcttcctcctggaagccacaagattaggc
cgagagccgatcagacaaacaacctacaacccttaagctcctggcagcgc
ccagccaaggccatgcttccttgcaacactccttccaaatggccatccca
gcatgcttccaagcaggcttcatccgttcctctggaccctcatctcttaa
gacctgccgcctataaaaaggattatatcttgagaccctatcctctaaaa
ttttttccacacccaaaacaaaaaatctctgggtcaaaagtctaaaacgc
ttaggctggcaaccatcagatccttgcccatggtgtcctcaagcctactc
tcatgaaatggacaacagtacacgcatatggggccagttccacatatttg
gcaaccagaccagcatccaggacaacacaaagtatgttgtttgttgttag
agggcttgggacatttcactctttgccagcctcagcttaatccaggagac
aaagattattttccttattatctcttctgcataggatctgcaatcagaac
tattgaacttctccattcagaccgccactcacacctatgggaaaagggta
atgtatcatcggcttagcaacagggaatactattcgtatgatggaaaatg
gggacaaaaggctttggtacataaaacattattccttccttggcctaaaa
actcatcgccacctacattaaagctaatatgcctgattactgtttttaga
gaacttattttattagggcagttccaagctcaaaaatacgctaactggca
ccttgttagctacataaaaatgcaccctagacccgaaacttactagactc
attataaaattttctttaaggtgtccacgcagtccctggtcacacttgaa
gcagtccggagaaatatcagccctaccccagtaatccccagaaggaactt
acacttttttttaatcttttcctacaacttcatattttataaataaaaag
acaaaaatgtcaggcctgtgagctgaagcttagccattgtaacccctgtg
acctgcacatatccgtccaggtggcctgcaggagccaagaagtctggagc
agccgaaaaaccacaaagaagtgaaacagccagttcctgccttaactaat
taacccaccttacgacattccaccattatgacttgtccaccattatgact
tgttcctgccctgccccaactgatcaatcaaccctgtgacattcttctcc
tggacaatgagtcccatcatctctccaccatgcaccttgtgaccccctcc
tctgctgaggataaccacctttaactgtaactttccacgcctacccaagc
cctataaagctgcccctctcctatctcccttcactgactctcttttcgga
ctcagcccacttgcacccaagtgaattaacagccttgttgctcacacaaa
gcctgattgggtgtcttctatacggacacgcgtgacaggaacctcaaccc
aaaggcagtctgatgaggtgtctaagataaaagtagcggcacaaaggctt
ttgtaaacagaggcgtttcatgtggttttcctttcctttccttatatgtg
aaaaggtgacagaaaagaaatcttcctaaaagagtc

chr19_53_399.b Amino Acid Sequence

(SEQ ID NO:16)

CCPIASEAPWTITDAELRVTLTVEDSQPYEDTLAGRSVLVKSLTPQTLQP
QWTRPYPVIYSTFTAVHLQDPLHWVHHSRIKPCPSDSQLDLSSSSWKPQD

### Example 2

### Identification of Candidate Genes 14

Four DNA sequences were identified as being overexpressed in colon carcinoma using the GENE LOGIC® (Gaithersburg, Md.) Gene Express Oncology datasuite. The sequences were identified in a datasuite search, which compared gene expression in colon tumors with expression in normal tissues. These sequences represent genes and encode antigens which are targets for colon cancer therapeutics.

The nucleotide sequences of each candidate gene are listed below. The first sequence, listed for each candidate gene was obtained directly from the public NCBI database (www.ncbi.nlm.nih.zov) and corresponds to the GenBank Accession No. number listed in the GENE, LOGIC® database. Additional sequence information was obtained by sequencing EST clones corresponding to each candidate gene.

Candidate 1: GenBank Accession No. W91975
W91975/IMAGE Clone 415310 3′ mRNA Sequence

(SEQ ID NO:17)

GGCTTCTAAGGTACATTATGTTTTACTTTAATAAATAAAAATTAACTTGA
AGAAAAATGCAGNGCCCTATTTAATTGCTCTGCATGAAATGTACAGAAAC
GGCAACCTCTGCGATTCTAAGCACTGTGAACGCCCCAGCCACACCGTGTC
AACAAACCGTGTGGCACTTGGGAGAAGGCAGGGGTGATTTACGANTAGTC
ATGTTTCGCCTCCACCCGAGTCACTGCCAAGGAGTGGACAGTGACACTGA
ATAAGCATNCGGNGCACCTCCTTCGGGAAGGGACTTGGCTGACATGGTAG
GCCTTCCCACTGGAGCCTGTACTTTGTCTTGCTGGGCAGCACTCCANTCA
TGGGAAGGAACAATGANCAAGGCGTGGTGGTGGGGGTGNGTAGGCCTGAG
CGCCGTTTTCCATGGTGACCTTCACTGAGCAGGCAGCAGGCACTGATGGG
CAGTTGAGNCTGGNAGGAGTCAGGTCCTGGTCNTGCCTCTGGTGTAACGC
AGCANGCCATCAAAGGT

IMAGE Clone 194681 T3 & T7 Consensus Sequence

(SEQ ID NO:18)

AGAATTCGGCACGAGNTTTTTTTTCTCTTAGATCTCCAGGTTCCCTTCCT
TACCCCGGGAAGCCTTTCTTCATCCCACCGTCCTGGGGCGTTNCACAGTG
CTTAGAATCGCAGAGGTTGCCGTTTCTGTACATTTCATGCAGAGCAATTA
AATAGGGCACTGCATTTTTCTTCAAGTTAATTTTTATTTATTAAAGTAAA
ACATAATGTACCTTAGAAGCCAGACAGTCCTACAAGCTTATTATGTTGTA
CAGCGGCGTTCCGTCCCCCTCCCCAGCCCTCTCTTTCTAGAGGCAGCCAA
TTTCAGCTGTCTCTCTCTGCTTACCTACATATTTCCATGTTTCTTGGTTC
ATCACCTGGTGGCACCTTCAGTCTGGAAACACCTGCCCTTCACTTTAGGG
GAATTGGGCCCCTGTTCGTTTGATAAGTTTTCCTACCATTTTCTGATTTG
TTTTTTCTTTCTGGAAAATGTATTAGTCAGATGTAGGCTTTTCTGGATTA
ATCCTTCAACTTTCCTTTCTTTCTTTCCCTTCCTGCCTGTCTCCCTGTTC
TTTCTTACACTTTCTCAGGGAGATTCTTGACTGTATTTTCCAACTTTGTA
TCGACCATTTTACTTTTCCTGCCATATTTTCAATGTTTACTGATGTTTCT
CTGCCCTTTCAGTGCATCCTGGTTTTATTTCATGTTAGACTGAATCCATG
TGAAATTGATAACAGGTTTTCAGCCCACACACACACACACAAAAAAAAAA
AAAAAAAAAAAAAAA

Candidate 2: GenBank Accession No. A1694242
A1694242/IMAGE Clone 2327838 3′ mRNA Sequence

(SEQ ID NO:19)

TTTTGTTGGCTGAGGCGGTATTTTCCTTTTATTGCTGTTATGAGATTCAA
CATTTTTTCCAGAAATAACTTCTGAAAAGTGTGCCTAGATTTTGAACACT
TGTGATCCTAACATGTGGTGAGAAAGGCTTTTCAAAACACACACGTGTGG
ACAGAGGTCCACACACGGATACGTGTGCACACACGGGTGCCTTGGGCGTG
CGTCTTCCAAAAGGGGCGAGTACAGCTATCAACTTGTGACTTCCAGGAGG
CCTGGGTTTGCCTACGAAGGGGCCGTGTTCCCAGTTGGCGTTCACACGTG
GTGTACACACACAGGCACAGGCACCGTGTCCCAAGGCCATCTCCCAAGGG
CACCCGCAGACACTGGGCAGCCTTCTCCGAAGCTGTCAGTGTCCTTCCTC
GTGAGAGGATGATGAAGAGGATGTGGTTTCCGCCGCCTCATCCACAGGCC
GGCTG

IMAGE Clone 2327838 T3 & T7 Consensus Sequence

(SEQ ID NO:20)

NAAAANGGCGCCNGNCCCANNTAAAATNNACCCNCCTAAAGGGGAAAAAC
TNNGGCGGCCGCCTTCGTTTTTTTTTTTTTTTTTTTGTGGTGGCTGAGGC
GGTATTTTCCTTTTATTGCTGTTAAGAGATTCAACATTTTTTCCAGAAAT
AACTTCTGAAAAGGGGGCCTNAGATTTTGAACACTTGGGATCCTAACAGG
GGGTGAGAAAGGCTTTTCAAAACACACNACGGGTGGACAGAGGTCCACAC
ACGGNATACGGGGGCACACACGGGTGCCTTGGGCGTGCGTCTTCCAAAAG
GGGCGAGNTACAGCTATCAACTTGTGACTTCCAGGAGGCCTGGGTTTGCC
TACGAAGGGGCCGNTGTTCCCAGTTGGCGTTCACACGTGGTGTACACACA
CAGGCACAGGCACCNGTGTCCCAANGGCCATCTNCCCAAGGGCACCCGCA
GACACTGGGCAGCCTTCTCCGAAGCTGTCAGTGTCCTTCCTCGTGAGAGG
ATGATGAAGAGGATGTGGTTTCCGCCGCCTCATCCACAGGCCGGCTGCCC
ACGGAGCCTTAGACATCGAGGCCAGAGCGACAGAAGCCTGTGTGCTGACC
GGCCTGGTCTCCTTTGACGTCTCGAGCAGCTTGGCAGGGTGGGAAAAGTA
GCCTGAGAGTGATCCCCGGGCAGTGTCCGAGGCTCTGCCGTCCCCACCCC
CACAGGCATCCAGGGGAGAGAAACAACCTGCGCCTGCGAGGCCGTGCGGA
CCCCGCTCCACTCACCCCGCCTGGGGGGCCAGAACCACCTCCCAGGGGCT
TCCGCCAGTGCCGCAGTTGCTGACCCCAGGCAAACCTCGCCGCCTCCTGC
CCCGGCGGGCCTGGGATTTGCGAATGTGTGAAGGCATTAGCTGCCAGTTG
TAACTGGAACCCAGCCTAGAGGCCTCACTCCTCCAGCAGGAAGCCTTGTA
ATGCAGCGAATCTGAACCCGGCCCAGCGTCCAGAGACAGGAAGCATTAAT
AGGAGCGAATGTGAACACTGTTCGCGCCCTGGCTGCGATTTATTGCCGAT
TGTGGGGAAAACATCAGTTGGTTGCAGAGTTTCATTCATCTTTAGGGACA
GGACCGGTGTGTCTGGGTGGCAGTTTAGAGAGCTGGGACAGTCGGCATCA
CTCTGGGTGGCTCCTCTCAANCCCTGGTGCCTCGTGCCGAATTCTGGCCT
CGAGGCATTCTNAGGGGCTNTATNC

Candidate 3: GenBank Accession No. AI680111
AI680111/IMAGE Clone 2252029 3′ mRNA Sequence

(SEQ ID NO:21)

TTTTTTTTTTTTGTGGATAAATATATTAGCAAATGAATATATTTCTTAAC
ATAGTGCCTGATTCAAGCGTCTGTCTGGTTCAAATATAAATACCCATGTG
GGTACCTAGGTGCTAGTCTCCCCACTAACTGAGGGAAAAAGGTTCCCAGG
TGGGGTCCTCTGCCCACTTTGCCACCACATTCACATTCCAAATGGGATAA
TGCCTGAGGGGCCATGAGTGGTCAGGCTGCCCTGGGGTGAATGTCACCCT
GATGAGGCCCATCAGCTCTTGTCCACTCAGTGAGGCCAGACTTGTGCTCT
AATCCACT

IMAGE Clone 2324560 T7 Sequence

(SEQ ID NO:22)

CTNTGTANAAAGCTGGGTACGCGTAAGCTTGGGCCCCTCGAGGGATACTC
TAGAGCGGCCGCCCTTTTTTTTTTTTTTTGTGGATAAATATATTAGCAAA
TAAATATATTTCTTAACATAGTGCCTGATTCAAGCGTCTGTCTGGTTCAG
ATATAAATACCCATGTGGGTACCTAGGTGCTAGTCTCCCCACTAACTGAG
GGAAAAAGGTTCCCAGGTGGGGTCCTCTGCCCACTTTGCCACCACATTCA
CATTCCAAATGGGATAATGCCTGAGGGGCCAAGAGTGGTCAGGCTGCCCT
GGGGTGAATGTCACCCTGATGAGGCCCATCAGCTCTTGTCCACTCAGTGA
GGCCAGACTTGTGCTCTAATCCACTCTCCTGTGGGTCCCTGGCCTGTATG
GCTTATACTGGGGAGCTGGGCCTCTGGGCTGTCCAAACCCAAGGGTCACA
CTTTGCTTTTCCTTTGTTGTCCCCATTTTCCATCCTTGCTCTAAGACAAA
ACTTTTCCCAGAGAAGAACTCTTTGTTGTCCCCGCTCAGCTGTAATTCTG
CCTTTTCTACCTTCATTCCATCCTTCCTCTGCCCAGATAAAGTCCAGCAG
AAATTCCTCCTTTCTACCTCTCTGGGACTCTGAGACAGGAAATCTTCAAG
GAGGAGTTTTTCCCTCCCCACTATTCTTATTCTCAACCCCCAGAAGAACC
AANGGCTGCTGTACCCCCCTCAGGGACAGAACTCCACACTATANGGGGGA
AAGNTTCANGGGACCCCTTCCTTTTANTGCTCANGGCTCCACCTATGCTA
CTGGNTCCTTTTGGCAAAAAAGGNAAATGANAGAGCCAGGGGTTGCCCCN
TGATGTAACANCCNTTACTGGGGANGGGNCCAANGNNGGTGNTCAAAGNN
CCCCNAGGAGGGAGGNGANAAGGGGTCATGNGTTCTGCTNAANCCNCTGG
TTGGTATAAANTTGANGNTTGGGGTGANGGAAACCAAAAANGGNTGGAAA
AAGNAAAACACCTTTNNAAACCCTGGGTACCNNANATAAGNTTTTGGCCC
NAAAAANTCNGCCNNCAAGGGATCCGCCCCNCCCCCCCAGGGAAAAANTT
GGTTCCTNGGGNGAAAAGGANTTTNCCCCCCNCAAATTTTNNCCNAAAAG
NTTTGGAANTTGNAAAANAAAAGGANCCTTCCCCCCCCCNCCACAAAAAA
AAAAAAAAAAAA

IMAGE Clone 2324560 SP6 Sequence

(SEQ ID NO:23)

CNNTTNCAAAAAGCAGGCTGGTACCGGTCCGGAATTCCCGGGATATCGTC
GACCCACGCCGTCCGGTTTGCTGGTGTTGCTGAAATAACTCCAGCAGAAG
GAAAATTAATGCAGTCCCACCCGCTGTACCTGTGCAATGCGAGTGATGAC
GACAATCTGGAGCCTGGATTCATCAGCATCGTCAAGCTGGAGAGTCCTCG
ACGGGCCCCCCGCCCCTGCCTGTCACTGGCTAGCAAGGCTCGGATGGCGG
GTGAGCGAGGAGCCAGTGCTGTCCTCTTTGACATCACTGAGGATCGAGCT
GCTGCTGAGCAGCTGCAGCAGCCGCTGGGGCTGACCTGGCCAGTGGTGTT
GATCTGGGGTAATGACGCTGAGAAGCTGATGGAGTTTGTGTACAAGAACC
AAAAGGCCCATGTGAGGATTGAGCTGAAGGAGCCCCCGGCCTGGCCAGAT
TATGATGTGTGGATCCTAATGACAGTGGTGGGCACCATCTTTGTGATCAT
CCTGGCTTCGGTGCTGCGCATCCGGTGCCGCCCCCGCCACAGCAGGCCGG
ATCCGCTTCAGCAGAGAACAGCCTGGGCCATCAGCCAGCTGGCCACCAGG
AGGTACCAGGCCAGCTGCAGGCAGGCCCGGGGTGAGTGGCCAGACTCAGG
GAGCAGCTGCAGCTCAGCCCCTGTGTGTGCCATCTGTCTGGAGGGAGTTC
TCTGAGGGGGCAGGAGCTACGGGTCATTTCCCTGCCTCCATGAGTTCCAT
CGTAACTGTGTGGACCCCTGGNTACATCAGCATCCGGACTTGCCCCCTCT
TGCATGGTTCAACATCACANAGGGGAGATCCNTTTTCCCNGTCCCTGGGA
ACCTCTNCNATCTTACCAAGAACCAGGGTCGGAAGACTCCCCCCTCATTT
CNCCAGCATCCCCGGCATGNCCCACTACACCNTCCCTGGTNGCCTACCTG
TTNGGGCCCTTCCCCGGAATGCAGGGGNTNGGGCCCCCNCNAACTGGGTC
CTTTCCTGCCNTCCAGGNAGCCAGGCATGGGCCCCCCGAATCACCCCTTC
CCCNAANATGGANNATCCCCCGGGTTCCAGGAAAACAAACAACCNCTGGA
AGGAANCCNNNACCCCNTNNCCCNAAGGCTGGGGAANGNAACNCCCCCNA
TTCCCCNTNNANGANCCCTNNGTTTNCNCNAGGCCCCTNACCCGGGCCNN
GCCCCCNAAACAAAGGGANTTGANAAANT

These sequences correspond to hypothetical gene FLJ20315/GenBank Accession No. No. AK000322.

AK000322 Nucleotide Sequence

(SEQ ID NO:24)

AAAAAAAAAAAACTTTAGAGAAAGGAAGGGCCAAAACTACGACTTGGCTT
TCTGAAACGGAAGCATAAATGTTCTTTTCCTCCATTTGTCTGGATCTGAG
AACCTGCATTTGGTATTAGCTAGTGGAAGCAGTATGTATGGTTGAAGTGC
ATTGCTGCAGCTGGTAGCATGAGTGGTGGCCACCAGCTGCAGCTGGCTGC
CCTCTGGCCCTGGCTGCTGATGGCTACCCTGCAGGCAGGCTTTGGACGCA
CAGGACTGGTACTGGCAGCAGCGGTGGAGTCTGAAAGATCAGCAGAACAG
AAAGCTGTTATCAGAGTGATCCCCTTGAAAATGGACCCCACAGGAAAACT
GAATCTCACTTTGGAAGGTGTGTTTGCTGGTGTTGCTGAAATAACTCCAG
CAGAAGGAAAATTAATGCAGTCCCACCCACTGTACCTGTGCAATGCCAGT
GATGACGACAATCTGGAGCCTGGATTCATCAGCATCGTCAAGCTGGAGAG
TCCTCGAGGGGCCCCCCGCCCCTGCCTGTCACTGGCTAGCAAGGCTCGGA
TGGCGGGTGAGCGAGGAGCCAGTGCTGTCCTCTTTGACATCACTGAGGAT
CGAGCTGCTGCTGAGCAGCTGCAGCAGCCGCTGGGGCTGACCTGGCCAGT
GGTGTTGATCTGGGGTAATGACGCTGAGAAGCTGATGGAGTTTGTGTACA
AGAACCAAAAGGCCCATGTGAGGATTGAGCTGAAGGAGCCCCCGGCCTGG
CCAGATTATGATGTGTGGATCCTAATGACAGTGGTGGGCACCATCTTTGT
GATCATCCTGGCTTCGGTGCTGCGCATCCGGTGCCGCCCCCGCCACAGCA
GGCCGGATCCGCTTCAGCAGAGAACAGCCTGGGCCATCAGCCAGCTGGCC
ACCAGGAGGTACCAGGCCAGCTGCAGGCAGGCCCGGGGTGAGTGGCCAGA
CTCAGGGAGCAGCTGCAGCTCAGCCCCTGTGTGTGCCATCTGTCTGGAGG
AGTTCTCTGAGGGGCAGGAGCTACGGGTCATTTCCTGCCTCCATGAGTTC
CATCGTAACTGTGTGGACCCCTGGTTACATCAGCATCGGACTTGCCCCCT
CTGCGTGTTCAACATCACAGAGGGAGATTCATTTTCCCAGTCCCTGGGAC
CCTCTCGATCTTACCAAGAACCAGGTCGAAGACTCCACCTCATTCGCCAG
CATCCCGGCCATGCCCACTACCACCTCCCTGCTGCCTACCTGTTGGGCCC
TTCCCGGAGTGCAGTGGCTCGGCCCCCACGACCTGGTCCCTTCCTGCCAT
CCCAGGAGCCAGGCATGGGCCCTCGGCATCACCGCTTCCCCAGAGCTGCA
CATCCCCGGGCTCCAGGAGAGCAGCAGCGCCTGGCAGGAGCCCAGCACCC
CTATGCACAAGGCTGGGGAATGAGCCACCTCCAATCCACCTCACAGCACC
CTGCTGCTTGCCCAGTGCCCCTACGCCGGGCCAGGCCCCCTGACAGCAGT
GGATCTGGAGAAAGCTATTGCACAGAACGCAGTGGGTACCTGGCAGATGG
GCCAGCCAGTGACTCCAGCTCAGGGCCCTGTCATGGCTCTTCCAGTGACT
CTGTGGTCAACTGCACGGACATCAGCCTACAGGGGGTCCATGGCAGCAGT
TCTACTTTCTGCAGCTCCCTAAGCAGTGACTTTGACCCCCTAGTGTACTG
CAGCCCTAAAGGGGATCCCCAGCGAGTGGACATGCAGCCTAGTGTGACCT
CTCGGCCTCGTTCCTTGGACTCGGTGGTGCCCACAGGGGAAACCCAGGTT
TCCAGCCATGTCCACTACCACCGCCACCGGCACCACCACTACAAAAAGCG
GTTCCAGTGGCATGGCAGGAAGCCTGGCCCAGAAACCGGAGTCCCCCAGT
CCAGGCCTCCTATTCCTCGGACACAGCCCCAGCCAGAGCCACCTTCTCCT
GATCAGCAAGTCACCGGATCCAACTCAGCAGCCCCTTCGGGGCGGCTCTC
TAACCCACAGTGCCCCAGGGCCCTCCCTGAGCCAGCCCCTGGCCCAGTTG
ACGCCTCCAGCATCTGCCCCAGTACCAGCAGTCTGTTCAACTTGCAAAAA
TCCAGCCTCTCTGCCCGACACCCACAGAGGAAAAGGCGGGGGGGTCCCTC
CGAGCCCACCCCTGGCTCTCGGCCCCAGGATGCAACTGTGCACCCAGCTT
GCCAGATTTTTCCCCATTACACCCCCAGTGTGGCATATCCTTGGTCCCCA
GAGGCACACCCCTTGATCTGTGGACCTCCAGGCCTGGACAAGAGGCTGCT
ACCAGAAACCCCAGGCCCCTGTTACTCAAATTCACAGCCAGTGTGGTTGT
GCCTGACTCCTCGCCAGCCCCTGGAAGCACATCCACCTGGGGAGGGGCCT
TCTGAATGGAGTTCTGACACCGCAGAGGGCAGGCCATGCCCTTATCCGCA
CTGGCAGGTGCTGTCGGCCCAGCCTGGCTCAGAGGAGGAACTCGAGGAGC
TGTGTGAACAGGCTGTGTGAGATGTTCAGGCCTAGCTCCAACCAAGAGTG
TGCTCCAGATGTGTTTGGGCCCTACCTGGCACAGAGTCCTGCTCCTGGGA
AAGGAAAGGACCACAGCAAACACCATTCTTTTTGCCGTACTTCCTAGAAG
CACTGGAAGAGGACTGGTGATGGTGGAGGGTGAGAGGGTGCCGTTTCCTG
CTCCAGCTCCAGACCTTGTCTGCAGAAAACATCTGCAGTGCAGCAAATCC
ATGTCCAGCCAGGCAACCAGCTGCTGCCTGTGGCGTGTGTGGGCTGGATC
CCTTGAAGGCTGAGTTTTTGAGGGCAGAAAGCTAGCTATGGGTAGCCAGG
TGTTACAAAGGTGCTGCTCCTTCTCCAACCCCTACTTGGTTTCCCTCACC
CCAAGCCTCATGTTCATACCAGCCAGTGGGTTCAGCAGAACGCATGACAC
CTTATCACCTCCCTCCTTGGGTGAGCTCTGAACACCAGCTTTGGCCCCTC
CACAGTAAGGCTGCTACATCAGGGGCAACCCTGGCTCTATCATTTTCCTT
TTTTGCCAAAAGGACCAGTAGCATAGGTGAGCCCTGAGCACTAAAAGGAG
GGGTCCCTGAAGCTTTCCCACTATAGTGTGGAGTTCTGTCCCTGAGGTGG
GTACAGCAGCCTTGGTTCCTCTGGGGGTTGAGAATAAGAATAGTGGGGAG
GGAAAAACTCCTCCTTGAAGATTTCCTGTCTCAGAGTCCCAGAGAGGTAG
AAAGGAGGAATTTCTGCTGGACTTTATCTGGGCAGAGGAAGGATGGAATG
AAGGTAGAAAAGGCAGAATTACAGCTGAGCGGGGACAACAAAGAGTTCTT
CTCTGGGAAAAGTTTTGTCTTAGAGCAAGGATGGAAAATGGGGACAACAA
AGGAAAAGCAAAGTGTGACCCTTGGGTTTGGACAGCCCAGAGGCCCAGCT
CCCCAGTATAAGCCATACAGGCCAGGGACCCACAGGAGAGTGGATTAGAG
CACAAGTCTGGCCTCACTGAGTGGACAAGAGCTGATGGGCCTCATCAGGG
TGACATTCACCCCAGGGCAGCCTGACCACTCTTGGCCCCTCAGGCATTAT
CCCATTTGGAATGTGAATGTGGTGGCAAAGTGGGCAGAGGACCCCACCTG
GGAACCTTTTTCCCTCAGTTAGTGGGGAGACTAGCACCTAGGTACCCACA
TGGGTATTTATATCTGAACCAGACAGACGCTTGAATCAGGCACTATGTTA
AGAAATATATTTATTTGCTAATATATTTAT

The hypothetical protein encoded by this sequence is listed under GenBank Accession No. BAA91085, provided below:

BAA91085 Amino Acid Sequence

(SEQ ID NO:25)

MSGGHQLQLAALWPWLLMATLQAGFGRTGLVLAAAVESERSAEQKAVIRV
IPLKMDPTGKLNLTLEGVFAGVAEITPAEGKLMQSHPLYLCNASDDDNLE
PGFISIVKLESPRRAPRPCLSLASKARMAGERGASAVLFDITEDRAAAEQ
LQQPLGLTWPVVLIWGNDAEKLMEFVYKNQKAHVRIELKEPPAWPDYDVW
ILMTVVGTIFVIILASVLRIRCRPRHSRPDPLQQRTAWAISQLATRRYQA
SCRQARGEWPDSGSSCSSAPVCAICLEEFSEGQELRVISCLHEFHRNCVD
PWLHQHRTCPLCVFNITEGDSFSQSLGPSRSYQEPGRRLHLIRQHPGHAH
YHLPAAYLLGPSRSAVARPPRPGPFLPSQEPGMGPRHHRFPRAAHPRAPG
EQQRLAGAQHPYAQGWGMSHLQSTSQHPAACPVPLRRARPPDSSGSGESY
CTERSGYLADGPASDSSSGFCHGSSSDSVVNCTDISLQGVHGSSSTFCSS
LSSDFDPLVYCSPKGDPQRVDMQPSVTSRPRSLDSVVPTGETQVSSHVHY
HRHRHHHYKKRFQWHGRKPGPETGVPQSRPPIPRTQPQPEPPSPDQQVTG
SNSAAPSGRLSNPQCPRALPEPAPGPVDASSICPSTSSLFNLQKSSLSAR
HPQRKRRGGPSEPTPGSRPQDATVHPACQIFPHYTPSVAYPWSPEAHPLI
CGPPGLDKRLLPETPGPCYSNSQPVWLCLTPRQPLEPHPPGEGPSEWSSD
TAEGRPCPYPHCQVLSAQPGSEEELEELCEQAV

Candidate 4: GenBank Accession No. AA813827
AA813827/IMAGE Clone 1271704 3′ mRNA Sequence

(SEQ ID NO:26)

TTTTTTTTTAAACATTAAGATTTTATTACAAACCAGGCATTATATATTTC
TTTACACTTAAGGAATAGATATGAAACAATCTTGGAGTAAAAATTAGAAG
GCAACTTGCTTCAAGTTTGTACCAAGTCAATCAAGCAGAAACCTGAAGAA
CCTTGTTTTAAGATGAGAGTCATTTATACTTGGCAGGCATTTTCTTCCAA
TGAAAAAATAAAGTCAATGTGCCATTATCTTGACACTTATAAAAATGTTT
ATAAAAAGCATTTAGGCCATTGATTCTCACAGTTGGCTGAATATTGGAAT
CACCTAGATTAAAAAAAATACTAATCCCTATACAACATCCCCAAAATTCA
GATTTAATTAGTGTAAGTTAGGCCCTGGGCATATAGGCTGTTTTAAAATT
CCTCGGGTGAGTCTAATGTGTA

IMAGE Clone 1341074 T7 Sequence

(SEQ ID NO:27)

CCCNNCNNCCNNNNNNGNNNNNCTTANCTCGCAGNCANAATTCGGCCACG
CAGGGTCGCCTTCGCCGCCATGGNACGCCACCGGGCGCTGACAGACCTAT
GGAGAGTCAGGGTGTGCCTCCCGGGCCTTATCGGGCCACCAAGCTGTGGA
ATGAAGTTACCACATCTTTTCGAGCAGGAATGCCTCTAAGAAAACACAGA
CAACACTTTAAAAAATATGGCAATTGTTTCACAGCAGGAGAAGCAGTGGA
TTGGCTTTATGACCTATTAAGAAATAATAGCAATTTTGGTCCTGAAGTTA
CAAGGCAACAGACTATCCAACTGTTGAGGAAATTTCTTAAGAATCATGTA
ATTGAAGATATCAAAGGGAGGTGGGGATCAGAAAATGTTGATGATAACAA
CCAGCTCTTCAGATTTCCTGCAACTTCGCCACTTAAAACTCTACCACGAA
GGTATCCAGAATTGAGAAAAAACAACATAGAGAACTTTTCCAAAGATAAA
GATAGCATTTTTAAATTACGAAACTTATCTCGTAGAACTCCTAAAAGGCA
TGGATTACATTTATCTCAGGAAAATGGCGAGAAAATAAAGCATGAAATAA
TCAATGAAAGATCAAGAAAATGCAATTGATAATAGAGAACTAAGCCAGGA
AGATGTTGAAAGAAGNTTGGGAGATATGTTATTCTGATCCTACCTGCAAA
CCATTTTAAGGTGTGCCCATCCCCTAGAAGNAAGTTCTTAAATCCCAAAC
CAGGTAATTCCCCCAANTANTTAATGNACAAACATGGNCCAATACAAGTT
AANCCNGGGAGTAGTTNTTACTACAAAACCAATTCNGATGACCTTCCCCC
ACNGGNTNTTTNNCTNGCCATGGAAANGNCCCTACCAAANTGGCCCAANA
ANNCANTGATTTGGAATAATCCNNCCTTTGGTTGGGATTNNANCAAATTG
ANTCCNAANNATCCCCAAATANTTTNCNAAANNCTCCCTGANCCCNACCT
ANCTTTGGAANTTNCCCAATTNTTTGGCAAACNTTTTGGGGANGGAAAGA
ATTCTCCGGATTTNAGCCCTTNTGGCAAAGGNTNCACCTNNNTTNAATTT
NAAGANNNACACCCTNGGNAAATNTAANGGGGCCCCCNNATTNTTTNAAA
TNCGCGGAANAAGNTCCCAGGNTCCCNTNTTTCCCCCCAAAATNNNATTG
GGATTCCTNACCCCCCCAN

IMAGE Clone 1341074 T3 Sequence

(SEQ ID NO:28)

CNNNNNANTGCGGCCGCTCATTTTTTTTTTTTTTTTTTCTCTATGNAAGC
AGACTGNAGNAAGAAGGCACTCAGNTTGATTTGAAGGAATTCAAATTGTT
TAAGTGAAGGAATTTTGAAGACTGTGGATCATCTTGAATTTTATGTATCC
CACTGGATCTATCTGAAACTGTGATGTAGCCACAAACAACTACCAGGAAA
TGAAACAAAAATTAAGATGCAACTGTATGACAGTGGACAAAAATAAAACA
AAAACAATAGTAAAGTTAAAAAATAAAGCATTACTATAGTATATATTGTT
AGTATAGTATACACAGTAGTTGCTTAATTCAGAAGCCACTTAAATAGGAC
ACATGCAACATTCGGTTACAAACGTGCAAGACAGATGAGTGGTTTTCCCA
TTTGTAATATAACTTTAAAAAATTATTTCAACAGCCTAATTAAATGGATT
GAGCCAGAATACATTTAAAAAATCTGTTCTCAGTCTGCAAGTACTAGAAA
CCTCATAAATATAAGATAATTGTGGTATAATAAAATACATATATTTGATC
TTTGTCCTTGGTACCTGGTATGGAGCTCCTAAAATCCTTGAAATTTCCTG
AATGATAGAAGTCTTTAGTTACTCATAACAAGCCTATTTCAGCGNTATCC
TGAGTTTCATGCCTAANGGTAACTGANGGCCNGGCCATGGGTTTGAATTT
TCATCCACCAACTACAACCCTTGTGGGGAGGAGAAAGGGNCTAGAAATTN
AAGTTCNNTTGGNCCACCAGTGACCCAATGAATTGGGTCCNGTCATGCCT
TGGNTANTTAAACCTTCCAATTAAAACNCNTAAAACATGCNAGGCTGANG
GGAGTTTTNTAGGGTNNNGGAANCCTTGNATGGGGCTGGGNATCCCCGGA
TTGACCCAGAAANGGTAAAAAAAACNCTTNGGCCCCCCCCCCCCCCCTNA
CCCGGGGNCTTGGGAAACCCCTCCCTTTGGCCNTTTNCTGGAGGNCNACC
CTTTTNAAATAAACTAAAAGCCATAGNTAAAGGGGCNTTTTNCTNNTTNC
TGGGAANCTTGNANGGAATTTTTNGACCCNGGNAAGGGGNTTTGAGGGAA
ANCCCAANTNGGTAATTGGCNGGGCGGGAATTTNNATACCCCCNGAACCC
NATTNCNCGGAATTAAAAAAATTTNGGNNCGGNCCCCTTTNTNTNNNCCA
GGGGTNAAANTTCTCNAAANNANAAA

IMAGE Clone 1676529 T7 Sequence

(SEQ ID NO:29)

AGCTCGNAGCCAGATTCGGCACGAGGGAGATTATATGTTTTATTTATCAT
TGTCTCTGCATATCTGGAACAACGAAAGGCACATAGCAGTTGCTAAATAA
ATATCTTTTGAATGAATATATGATTGCCTTATACTTCTTTTATATCCCCA
TCTTCTAATAGATTATGAAAACTAGAATTCAAAATATATATACTGAACAA
ATGAATGACTGAAGCAATTGGGGATAATATTTAAGGCAAAACCAAATCTG
ATAAAATATACACATATTTTAAAAACACATACATATATATAAATAGATCA
AAAGTGGAAAAAGAATATATAAAAGAGTGCAACATTTGGCAGCTGAGAAT
TATTTCATTGAGTTTTCAAATATTCTTCACATTCTTATACTTAGAAACAA
AGAAGTAACCCCAAACAACTAATTCATTAGCTAATATCTCAGAACTTGCA
CATTTGCAGATAAATTTTCTTTTAAGAACAGAATTATAGTTTAATCCCTA
ACACAGCTCAGTTTTCAAAATTCAAGTAAATAAAATTTTAGCACACATCA
TGATAGCCTTACTGGNATAGCTGTGTTAAAAACAAAAAGTATTTGGTATC
ATCTATTGTTATGTGCTCTCAATTGAGATCTAGTTAGTTTCCTAAGAGTC
TCACATTGATANCTATTTTGGGCACTTCCTTACATAATGNGNTTATTTAG
AAATACCTTATTAATGACAGACTTCCTTTTGAGTAGCTACATTCTCAGAT
ATGGCTNCATTTATCAAAGTTCCCCNAGGATTACCTAATTTTAATTCCAG
TTAGNTATCTAAACTACGGAACTTTNGGNTTTCCTTAAANTCAACATTGG
TTGCCTTGATTGGAAGGNTTGGCNCCCAAAAANGGCGGNCNTCCCNCNCC
CGGGGGTGGNAANTCTTTTCNTGAANNTNCCAAGGNNAATTCCCTCCNGA
AANCNGGNTTTAANTTTTTTNCCNTTTCCCCCTTNAANGGGAAACCCCCG
GGTTTTNAAAAAAATTTTTCCCAAAANATTCNNCCNATGGGCCCCTTTGG
AAAGGNAAAAANTTTTTTGTCCCTTAAAAANCCCTGGNAACCNAATTTGG
TTNANCAAATANAGGAAGG

IMAGE Clone 167529 T3 Sequence

(SEQ ID NO:30)

GCGGCCGCTGGGCCTGNGTGTCGCCTTCGCCGCCATGGNCGCCACCGGGC
GCTGACAGACCTATGGAGAGTCAGGGTGTGCCTCCCGGGCCTTATCGGGC
CACCAAGCTGTGGAATGAAGTTACCACATCTTTTCGAGCAGGAATGCCTC
TAAGAAAACACAGACAACACTTTAAAAAATATGGCAATTGTTTCACAGCA
GGAGAAGCAGTGGATTGGCTTTATGACCTATTAAGAAATAATAGCAATTT
TGGTCCTGAAGTTACAAGGCAACAGACTATCCAACTGTTGAGGAAATTTC
TTAAGAATCATGTAATTGAAGATATCAAAGGGAGGTGGGGATCAGAAAAT
GTTGATGATAACAACCAGCTCTTCAGATTTCCTGCAACTTCGCCACTTAA
AACTCTACCACGAAGGTATCCAGAATTGAGAAAAAACAACATAGAGAACT
TTTCCAAAGATAAAGATAGCATTTTTAAATTACGAAACTTATCTCGTAGA
ACTCCTAAAAGGCATGGATTACATTTATCTCAGGAAAATGGCGAGAAAAT
AAAGCATGAAATAATCAATGAAGATCAAGAAAATGCAATTGATAATAGAG
AACTAAGCCAGGAAGATGTTGAAGAAGTTTGGGAGATATGTTATTCTGAT
CTACCTGCAAACCATTTTAGGTGTGCCATCCCTAGAAGAAGTCATAAATC
CCAAACAAGTAATTCCCCAATATATAATGTACNACATGGCCAATACANGT
AACGTGGGAGTAGTTATACTACAAACAAATCAGATGACCTCCCTCACTGG
GTATTATCTGCCATGAAGNGCCTAGCAAATNGGCCAGAAGCATGATATGN
AATAATCCACCTTTGNNGGATTTGACCGANATGTNTTNGAACATCCCGAT
TATTTCTAAACCCCTGACCNCTNNTACTTTGAAATNANAATTATTGNAAN
CTTTGGGNTGCTNCNCCCTTTAAAGGGGTGCCNCCAAGCCTNNGTTNGTG
NTGTTACTNCCCCCAANCGAAAAGNNCNCTTTATGGGTGNTNCCCAAGAA
CAATNTNN

These sequences correspond to hypothetical gene FLJ20354GenBank Accession No. No. AK000361.

AK000361 Nucleotide Sequence

(SEQ ID NO:31)

GTGCCGAGACTCACCACTGCCGCGGCCGCTGGGCCTGAGTGTCGCCTTCG
CCGCCATGGACGCCACCGGGCGCTGACAGACCTATGGAGAGTCAGGGTGT
GCCTCCCGGGCCTTATCGGGCCACCAAGCTGTGGAATGAAGTTACCACAT
CTTTTCGAGCAGGAATGCCTCTAAGAAAACACAGACAACACTTTAAAAAA
TATGGCAATTGTTTCACAGCAGGAGAAGCAGTGGATTGGCTTTATGACCT
ATTAAGAAATAATAGCAATTTTGGTCCTGAAGTTACAAGGCAACAGACTA
TCCAACTGTTGAGGAAATTTCTTAAGAATCATGTAATTGAAGATATCAAA
GGGAGGTGGGGATCAGAAAATGTTGATGATAACAACCAGCTCTTCAGATT
TCCTGCAACTTCGCCACTTAAAACTCTACCACGAAGGTATCCAGAATTGA
GAAAAAACAACATAGAGAACTTTTCCAAAGATAAAGATAGCATTTTTAAA
TTACGAAACTTATCTCGTAGAACTCCTAAAAGGCATGGATTACATTTATC
TCAGGAAAATGGCGAGAAAATAAAGCATGAAATAATCAATGAAGATCAAG
AAAATGCAATTGATAATAGAGAACTAAGCCAGGAAGATGTTGAAGAAGTT
TGGAGATATGTTATTCTGATCTACCTGCAAACCATTTTAGGTGTGCCATC
CCTAGAAGAAGTCATAAATCCAAAACAAGTAATTCCCCAATATATAATGT
ACAACATGGCCAATACAAGTAAACGTGGAGTAGTTATACTACAAAACAAA
TCAGATGACCTCCCTCACTGGGTATTATCTGCCATGAAGTGCCTAGCAAT
TGGCCAAGAAGCAATGATATGAATGATCCAACTTATGTTGGATTTGAACG
AGATGTATTCAGAACAATCGCAGATTATTTTCTAGATCTCCCTGAACCTC
TACTTACTTTTGAATATTACGAATTATTTGTAAACATTTTGGTTGTTTGT
GGCTACATCACAGTTTCAGATAGATCCAGTGGGATACATAAAATTCAAGA
TGATCCACAGTCTTCAAAATTCCTTCACTTAAACAATTTGAATTCCTTCA
AATCAACTGAGTGCCTTCTTCTCAGTCTGCTTCATAGAGAAAAAAACAAA
GAAGAATCAGATTCTACTGAGAGACTACAGATAAGCAATCCAGGATTTCA
AGAAAGATGTGCTAAGAAAATGCAGCTAGTTAATTTAAGAAACAGAAGAG
TGAGTGCTAATGACATAATGGGAGGAAGTTGTCATAATTTAATAGGGTTA
AGTAATATGCATGATCTATCCTCTAACAGCAAACCAAGGTGCTGTTCTTT
GGAAGGAATTGTAGATGTGCCAGGGAATTCAAGTAAAGAGGCATCCAGTG
TCTTTCATCAATCTTTTCCGAACATAGAAGGACAAAATAATAAACTGTTT
TTAGAGTCTAAGCCCAAACAGGAATTCCTGTTGAATCTTCATTCAGAGGA
AAATATTCAAAAGCCATTCAGTGCTGGTTTTAAGAGAACCTCTACTTTGA
CTGTTCAAGACCAAGAGGAGTTGTGTAATGGGAAATGCAAGTCAAAACAG
CTTTGTAGGTCTCAGAGTTTGCTTTTAAGAAGTAGTACAAGAAGGAATAG
TTATATCAATACACCAGTGGCTGAAATTATCATGAAACCAAATGTTGGAC
AAGGCAGCACAAGTGTGCAAACAGCTATGGAAAGTGAACTCGGAGAGTCT
AGTGCCACAATCAATAAAAGACTCTGCAAAAGTACAATAGAACTTTCAGA
AAATTCTTTACTTCCAGCTTCTTCTATGTTGACTGGCACACAAAGCTTGC
TGCAACCTCATTTAGAGAGGGTTGCCATCGATGCTCTACAGTTATGTTGT
TTGTTACTTCCCCCACCAAATCGTAGAAAGCTTCAACTTTTAATGCGTAT
GATTTCCCGAATGAGTCAAAATGTTGATATGCCCAAACTTCATGATGCAA
TGGGTACGAGGTCACTGATGATACATACCTTTTCTCGATGTGTGTTATGC
TGTGCTGAAGAAGTGGATCTTGATGAGCTTCTTGCTGGAAGATTAGTTTC
TTTCTTAATGGATCATCATCAGGAAATTCTTCAAGTACCCTCTTACTTAC
TAGACTGCTAGTGGATAATAACATCTTGACTACTTAAAAAAGGGACATAT
TGAAAATCCTGGAGATGGACTATTTGCTCCTTTGCCTAACTTACTCATAC
TGTAAGCAGATTAGTGCTCAGGAGTTTGATGAGCAAAAAGTTTCTACCTC
TCAAGCTGCAATTGCTAGAACTCTTTAGAAAATATTATTAAAATACAGGA
GTTTACCTTAAAGGAAAAAAAAAAAAACAAAAAAAAAAAAAAAAAA

The hypothetical protein encoded by this sequence is contained under GenBank Accession No. BAA91111, provided below:

BAA91111 Amino Acid Sequence

(SEQ ID NO:32)

MESQGVPPGPYRATKLWNEVTTSFRAGMPLRKHRQHFKKYGNCFTAGEAV
DWLYDLLRNNSNFGPEVTRQQTIQLLRKFLKNHVIEDIKGRWGSENVDDN
NQLFRFPATSPLKTLPRRYPELRKNNIENFSKDKDSIFKLRNLSRRTPKR
HGLHLSQENGEKIKHEIINEDQENAIDNRELSQEDVEEVWRYVILIYLQT
ILGVPSLEEVINPKQVIPQYIMYNMANNTSKRGVVILQNKSDDLPHHWLS
AMKCLANWPRSNDMNDPTYVGFERDVFRTIADYFLDLFEPLLTFEYYELF
VNILVVCGYITVSDRSSGIHKIQDDPQSSKFLNLNNLNSFKSTECLLLSL
LHREKNKEESDSTERLQISNPGFQERCAKKMQLVNLRNRRVSANDIMGGS
CHNLIGLSNMHDLSSNSKPRCCSLEGIVDVPGNSSKEASSVFHQSFPNIE
GQNNKLFLESKPKQEFLLNLHSEENIQKPFSAGFKRTSTLTVQDQEELCN
GKCKSKQLCRSQSLLLRSSTRRNSYINTFVAEIIMKPNVGQGSTSVQTAN
ESELGESSATINKRLCKSTIELSENSLLPASSMLTGTQSLLQPHLERVAI
DALQLCCLLLPPPNRRKLQLLMRMISRMSQNVDMPKLHDAMGTRSLMIHT
FSRCVLCCAEEVDLDELLAGRLVSFLMDHHQEILQVPSYLLDC

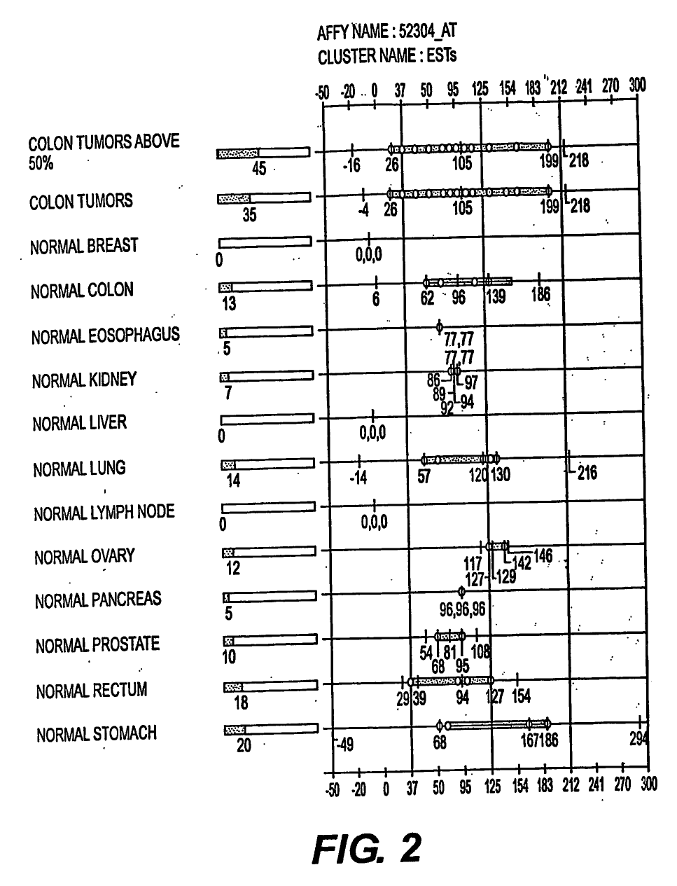

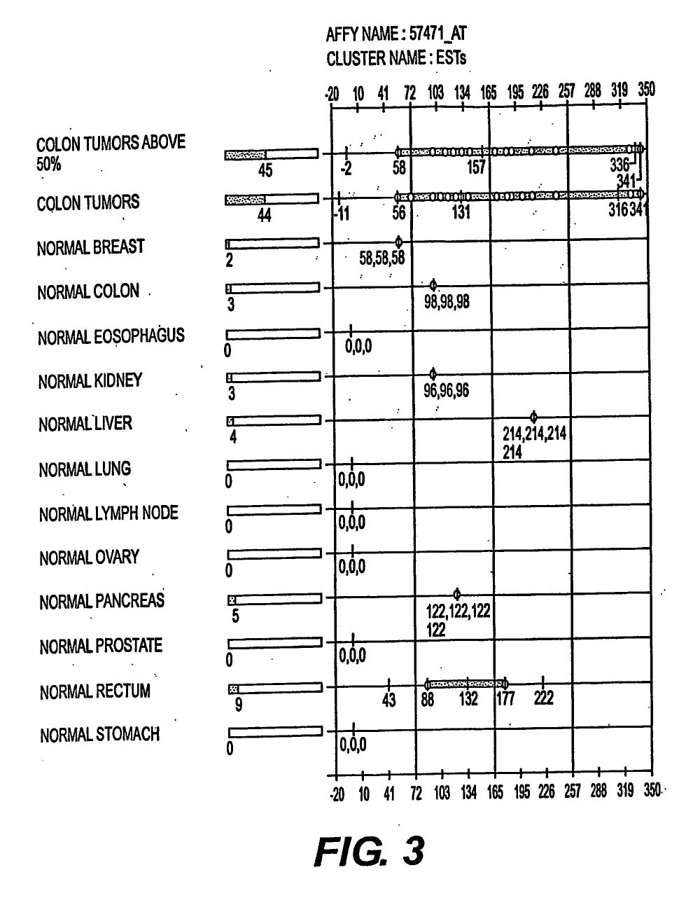

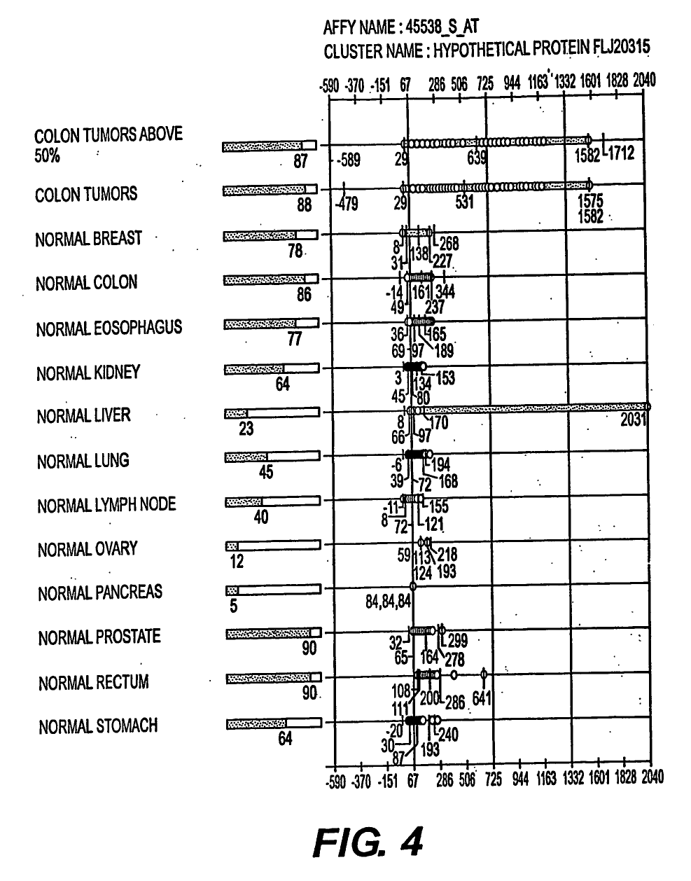

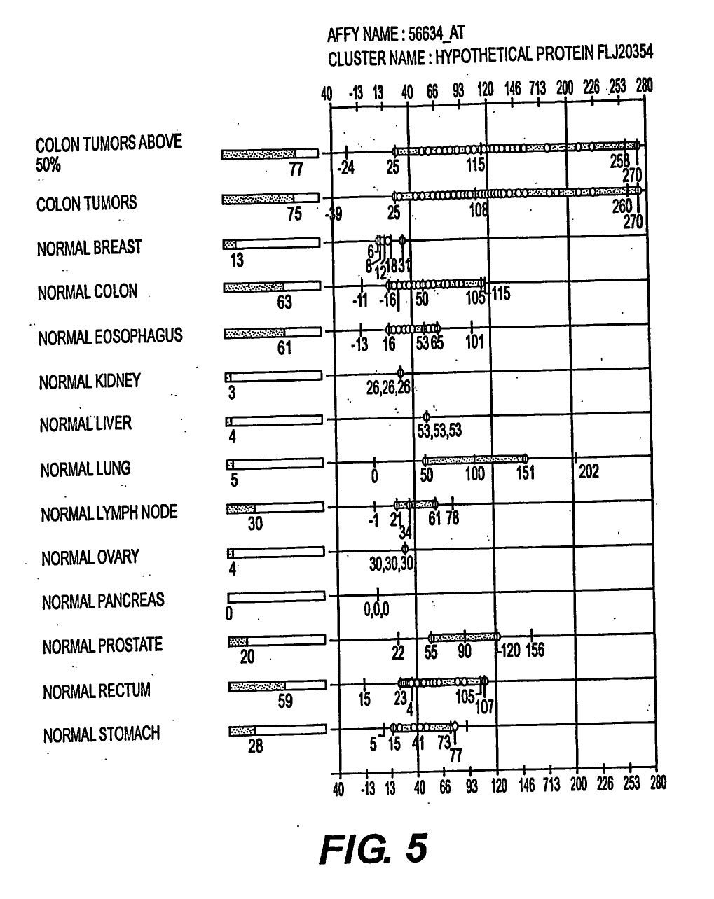

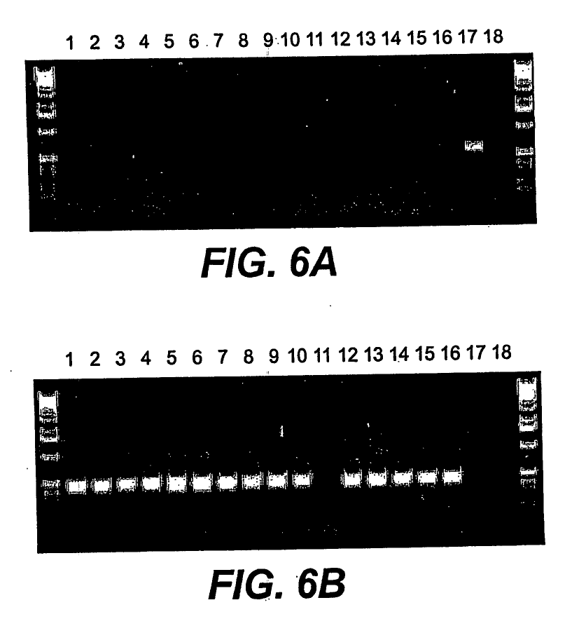

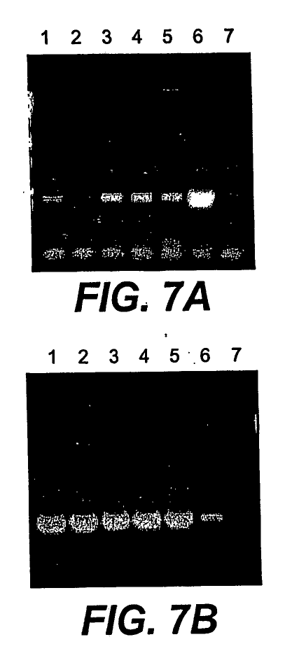

‘Electronic Northerns’ (E-Northerns) depicting gene expression profiles of the above described sequences were determined using the GENE LOGIC® Gene Express Oncology datasuite (Gaithersburg, Md.). See FIGS. 2-5. The expression of candidate 3 in normal and malignant human tissues was further investigated by PCR experiments using commercially available human cDNA panels and cDNA samples prepared in-house from human tissues and cell lines. See FIGS. 6A-6B and 7A-7B.

Expression of Glyceraldehyde 3-phosphate dehydrogenase (GAPDH) was measured in these experiments as a control for cDNA integrity. GAPDH is a housekeeping gene expressed abundantly in all human tissues. The following primers were used to amplify a 482 base pair product of the GAPDH gene:

| | |
|---|---|
| 5′ ACCACAGTCCATGCCATCAC 3′ | (SEQ ID NO:56) |
| 5′ TCCACCACCCTGTTGCTGTA 3′ | (SEQ ID NO:57) |

The following primers were used to amplify a 507 base pair product of the candidate 3 gene:

| | |
|---|---|
| 5′ TCCCACCCGCTGTACCTGTGC 3′ | (SEQ ID NO:58) |
| 5′ CCTGCAGCTGGCCTGGTACCT 3′ | (SEQ ID NO:59) |

Colon tumor samples were obtained from Grossmont Hospital in La Mesa, Calif. Colorectal cancer cell line HCT116 was obtained from the American Type Culture Collection (ATCC, Manassas, Va.). RNA was prepared from frozen tissue sections using the RNEasy® Maxi kit (Qiagen, #75162) or from fresh HCT116 cells using the RNEasy® Mini kit (Qiagen, #74104). For each sample, 2.5 μg RNA was first treated with DNAse I (Amplification Grade, Invitrogen #18068-015), then reverse transcribed using the SUPERSCRIPT® First Strand Synthesis System for RT-PCR (Invitrogen # 12371-019). For PCR, 1/25 of the reverse transcriptase (RT) reaction was used to screen for candidate 3, and 1/50 was used for GAPDH. The positive control for candidate 3 was IMAGE 2324560, obtained from the ATCC. The following primers were used to amplify a 415 base pair product of the candidate 3 gene:

(SEQ ID NO:60)

5′ GGAAGATCTGTTGAAGTGCATTGCTGCAGCTGGTAG 3′

(SEQ ID NO:61)

5′ CGCCATCCGAGCCTTGCTAGCCAG 3′

### Example 3

Using the same technology employed in Example 1 to identify the CICO genes, the following sequences were identified as differentially expressed in colon cancer:

bs421 ms433-258

At the +2 PCR stage, bs421 ms433-258 was found to be overexpressed in malignant colon compared to normal colon (FIG. 1). This peak was purified and amplified by PCR using the linkers with three additional nucleotides (+3 PCR). The +3 peaks were purified and sequenced.

bs421ms433-258 Nucleotide Sequence

(SEQ ID NO:33)

GATCTCACTCAGCAGACAGCAGCAGCCCGGGAGCCTGAGCTCAGGAGGAA
CTCTTACCTGGAAATTGGGAACTGTATGGAGACTCCAAACTGACTTCTTT
CAAAAAACAAAAACAAAAAATTTTTTTAGCTTTGACAAACACACAAAAGT
GGTAATAAAGAGAGCCCTCCTTGTCAACCCAAAATGTGAGCCCCCTGTGG
CAAAACCACCCCCTACCCCATTA

These bases correspond to the 3′UTR and some of the final coding exon of the hypothetical protein bK175E3.C22.6, the sequence of which is set forth below:

bK175E3.C22.6 Nucleotide Sequence

(SEQ ID NO:34)

cggccgcggggcccggcgcggcgcgggccaaggagacggcgttcgtggag
gtggtgctgttcgagtcgagcccaagcggcgattacaccacctacaccac
cggcctcacgggccgcttctcgcgggccggggccacgctcagcgccgagg
gcgagatcgtgcagatgcacccactgggcctatgtaataacaatgacgaa
gaggacttgtatgaatatggctgggtaggagtggtgaagctggaacagcc
agaattggacccgaaaccatgcctcactgtcctaggcaaggccaagcgag
cagtacagcggggagctactgcagtcatctttgatgtgtctgaaaaccca
gaagctattgatcagctgaaccagggctctgaagacccgctcaagaggcc
ggtggtgtatgtgaagggtgcagatgccattaagctgatgaacatcgtca
acaagcagaaagtggctcgagcaaggatccagcaccgccctcctcgacaa
cccactgaatactttgacatggggattttcctggctttcttcgtcgtggt
ctccttggtctgcctcatcctccttgtcaaaatcaagctgaagcagcgac
gcagtcagaattccatgaacaggctggctgtgcaggctctagagaagatg
gaaaccagaaagttcaactccaagagcaaggggcgccgggaggggagctg
tggggccctggacacactcagcagcagctccacgtccgactgtgccatct
gtctggagaagtacattgatggagaggagctgcgggtcatcccctgtact
caccggtttcacaggaagtgcgtggacccctggctgctgcagcaccacac
ctgcccccactgtcggcacaacatcatagaacaaaagggaaacccaagcg
cggtgtgtgtggagaccagcaacctctcacgtggtcggcagcagagggtg
accctgccggtgcattaccccggccgcgtgcacaggaccaacgccatccc
agcctaccctacgaggacaagcatggactcccacggcaaccccgtcacct
tgctgaccatggaccggcacggggagcagagcctctattccccgcagacc
cccgcctacatccgcagctacccacccctccacctggaccacagcctggc
cgctcaccgctgcggcctggagcaccgggcctactccccagcccacccct
tccgcaggcccaagttgagtggccgcagcttctccaaggcagcttgcttc
tcccagtatgagaccatgtaccagcactactacttccagggcctcagcta
cccggagcaggaggggcagtccccacctagcctcgcaccccggggcccgg
cccgtgcctttcctccgagcggcagtggcagcctgctcttccccaccgtg
gtgcacgtggccccgccctcccacctggagagcggcagcacgtccagctt
cagctgctatcacggccaccgctcggtgtgcagtggctacctggccgact
gcccaggcagcgacagcagcagcagcagcagctccggccagtgccactgt
tcctccagtgactctgtggtagactgcactgaggtcagcaaccagggcgt
gtacgggagctgctccaccttccgcagctccctcagcagcgactatgacc
ccttcatctaccgcagccggagcccctgtcgtgccagtgaggcggggggc
tcgggcagctcgggccggggacctgccctgtgcttcgagggctccccgcc
tcccgaggagctcccggcggtgcacagtcatggtgctgggcggggcgagc
cttggccgggccctgcctctccctcgggggatcaggtgtccacctgcagc
ctggagatgaactacagcagcaactcctccctggagcacagggggcccaa
tagctctacctcagaagtggggctcgaggcttctcctggggccgcccctg
acctcaggaggacctggaaggggggccacgagttgccgtcgtgtgcctgc
tgctgcgagccccagccctccccagccgggcctagcgccggagcagctgg
cagcagcaccttgttcctggggccccacctctacgagggctctggcccgg
cgggtggggagccccagtcaggaagctcccagggcttgtacggccttcac
cccgaccatttgcccaggacagatggggtgaaatacgagggtctgccctg
ctgcttctatgaagagaagcaggtggcccgcgggggcggagggggcagcg
gctgctacactgaggactactcggtgagtgtgcagtacacgctcaccgag
gaaccaccgcccggctgctaccccggggcccgggacctgagccagcgcat
ccccatcattccagaggatgtggactgtgatctgggcctgccctcggact
gccaagggacccacagcctcggctcctggggtgggacgcgaggcccggat
accccacggccccacaggggcctgggagcaacccgggaagaggagcgggc
tctgtgctgccaggctagggccctactgcggcctggctgccctccggagg
aggcgggtgctgtcagggccaacttccctagtgccctccaggacactcag
gagtccagcaccactgccactgaggctgcaggaccgagatctcactcagc
agacagcagcagcccgggagcctgagctcaggaggaactcttacctggaa
attgggaactgtatggagactccaaactgacttctttcaaaaaacaaaaa
caaaaaatttttttagctttgacaaacacacaaaagtggtaataaagaga
gccctccttgtcaacccaaaatgtgagccccctgtggcaaaaccaccccc
taccccattaacaaatcaacagacaaaattctccgagtcctttgcctctt
ttgataacatgttgttctgttttgtaaagtgtgtgtgcttggggttccga
ggtgtgggattgagttctctgctttgtttttttttaagatattgtatgta
aatgtaaaaagttatttaaatatatattttaaagaaccctaactgccaac
ttttgctgaaaaagaaaaaaaaatcactgctgcattaaatgaaccacatc
atgtgtagatactgttgtctccctgaagggagctcaggcctttgaaaagc
tcagggcttcacctgccttagaaaatgaaccagaaacttgaagtaaagct
agttgataggggtacaggctctgaggagcagtgcaaaactgcctctttct
ttctcgtggcaaatcccaatgtacacgatttcaggtctcagacgccatgc
ctctccagcccacgcctttaggcaggtgatggcagcagctaggaataggg
tgtacatgatccacagccctgcggagccaggtcaagccgctgctatgaaa
gctccagggtgatggggacgattctgcccagtgtcctcagtctgtcccct
caggtcatggtcccaagtgaaatgacagagttcacagccctggtcttggc
tgaggtccaggtcatagtaagggcatgttcttggggccctcgacctgaac
tctgaccctccgggcagggaagaggaggttgtcccctttggttgtcctgg
ctttggagtcctttgcaaaaatattttgggccccctgccactggctgcag
aaatggctcgacggggtgtgtggggacagacacccagaaggaatgtactt
ttgtggccttggtgtccgatggggctgggggagagtgctctccactgacc
cagcagcacacccatgtgcagtgcgcctgcatctgtgtgggggcagccac
accccttggctgctgcttccttgggctgcctttctgggggcatgtgactg
gacctacgaggtctgcactgagctccatttgaatgatacctttcctatcc
catttcccccacggaagcaccgcttcagggttattcagtcctctgcctca
tggctgaaattgctcatctcgtctgcagatgtctactatcctgtctacct
aatgcactattatgtattgattctccatgagacagagagagagagagact
atcagatagtttacacccaaagggtaggtttttgtatatttttccagcct
tttttattaaggggaaggggagagtttaaaaacccaaaccgttgtggttt
taaggtgtttcatttttaaaagggagagagaatctatttaaagctatttc
agatcagggattgtcatccttttttgtccaatgtattccttgttctttaa
aaaaattttttttagaggaaactaatattagtctttgtgttcactaactc
ttctggtcacttgtatttatttattcattcattcatcagatatttgttgc
catctgaaagaactggcccagtgggtctgaaagctcgcttgagaatagga
aacttgagacctggccccctgtgggtaggagaacaaggaccacctgggtt
ctccagtcttgaacgagaatctcactcttatcagaatgtttttcttaacc
tcagcgtatgatgaggaaatttacttatctctagctaggatttgacaaat
tccaacatcaaatgatcaaaacatttgccactgaggcttcactggtgaga
tccgttctccgtcctcgggtgcagtcccttgggggctgctcctcggactg
cgccccgcacacctgttatcgagggtgtgagaagcgcctaagctggtgac
atgtgatctgggacgccttcatttctcgggccaggagtagcagctgctaa
ggacagcagcttgcattgcgtggttttagggaagcagggtctggctttta
atatgaactgcaaaaagcagcttctcactgatatttttttgttgttgttt
ctggggggtttttttgttttgtttttaatgcctttgagtgcatattttct
tcctcgtctgaaaccgaactcccaaagtggctttctttagccctggctgg
aaaaccacctctcaatagccttaagcaataaatagatgagtagagaatgt
ggcttcaactgggcttattaaagtaagtgtgtctagttttcacttgaaca
agtgatagctgcagatggcgaaagaaacccatttaatttttgtagcttac
aggtggtagaaacaaaaatgcaattttaaaaccttaaataccaaatacca
accattgccttttttttttttgagatggaattttgctcttgtcacccagg
ctggagtgcaatggcgcgatctcacctcactgcaacctctgcctcccggg
tccaagtgattctcctgcctcagcctcccaagtagctgggattacaggca
tgcgccaccacacccagctaattttgtatttttggtagagacagggtatc
tccatgttggtcaggctggtcttggattcccgacctcaggtgatccgccc
acctcggcctcccaaagtgctgggattacaggcgtgagccaccatgcctg
cccagcaataccaaccattgtcttttaaattcgtgttggcttctcagaca
gggagatcactggaataaaataaccgatggtcttattttgtcacacgtaa
atcaaaagaaatgtcctctttgaagttgtaagactccaccaatgacagac
acccttttcggtggactctgagtggtgtgtagtggttttatagccatgga
aactaggagtatctcactttccactgagaacccctgcccccaatccctct
aagttggggtgtggcagttgggcagggtcaagtgacccagccctggctgt
aggacagccatatacagtgaagagttctagaaccagctaaaaatggaagt
ttgggtgtttaccaacaaggtacctctttatggatgcagccccagtaagc
tggctttaactctcagctccttccctgtctcctcctaatccaagcccttt
tataaaataaagccccttctgtcccactgctcacatacttatgtgctgct
agtctctactcgaagttcgtgcaggactaatgcttttaaaatgaggtcta
aaaaataattactagtcgagactattattctttaaacagaactgcctttt
tctactctttatgtaaactctttctattgtgttggtctaacaaggcacta
ttttaaaattttttaatttttcccatagcacttaaaagagattttgtaaa
gaccttgctgtaaagattttgtaataaaatggtctaagggctctttttcc
aacattaccatttttaaaaaatgttttaaaagctagaagacaacttatgt
atattctgtatatgtatagcagcacatttcatttatggaaatatgttctc
agaatatttatttactaatatatttatcttaagccatgtcttatgttgag
agtgtgacattgttggaataatcattgaaaatgactaacacaagaccctg
taaatacatgataattgcacacagattttacatatttgcagaccaaaaat
gatttaaaacaagttgtagtcttctatggttttgtaacaaattgtacaca
tgactgtaaaaaaaaaatacaattttatcaagtatgtgttata

The above sequence encodes the following protein:

bK175E3.C22.6 Amino Acid Sequence

(SEQ ID NO:35)

MHPLGLCNNNDEEDLYEYGWVGVVKLEQPELDPKPCLTVLGKAKRAVQRG
ATAVIFDVSENPEAIDQLNQGSEDPLKRPVVYVKGADAIKLMNIVNKQKV
ARARIQHRPPRQPTEYFDMGIFLAFFVVVSLVCLILLVKIKLKQRRSQNS
MNRLAVQALEKMETRKFNSKSKGRREGSCGALDTLSSSSTSDCAICLEKY
IDGEELRVIPCTHRFHRKCVDPWLLQHHTCPHCRHNIIEQKGNPSAVCVE
TSNLSRGRQQRVTLPVHYPGRVHRTNAIPAYPTRTSMDSHGNPVTLLTMD
RHGEQSLYSPQTPAYIRSYPPLHLDHSLAAHRCGLEHRAYSPAHPFRRPK
LSGRSFSKAACFSQYETMYQHYYFQGLSYPEQEGQSPPSLAPRGPARAFP
PSGSGSLLFPTVVHVAPPSHLESGSTSSFSCYHGHRSVCSGYLADCPGSD
SSSSSSSGQCHCSSSDSVVDCTEVSNQGVYGSCSTFRSSLSSDYDPFIYR
SRSPCRASEAGGSGSSGRGPALCFEGSPPPEELPAVHSHGAGRGEPWPGF
ASFSGDQVSTCSLEMNYSSNSSLEHRGPNSSTSEVGLEASPGAAPDLRRT
WKGGHELPSCACCCEPQPSPAGPSAGAAGSSTLFLGPHLYEGSGPAGGEP
QSGSSQGLYGLHPDHLPRTDGVKYEGLPCCFYEEKQVARGGGGGSGCYTE
DYSVSVQYTLTEEPPPGCYPGARDLSQRIPIIPEDVDCDLGLPSDCQGTH
SLGSWGGTRGPDTPRPHRGLGATREEERALCCQARALLRPGCPPEEAGAV
RANFPSALQDTQESSTTATEAAGPRSHSADSSSPGA

This protein contains a transmembrane domain as determined by SMART (rectangle), SOSUI, and TmPred. SMART also predicts that this protein contains a RING domain (triangle), which is a zinc finger domain involved in protein-protein interactions. The structure of the protein is depicted schematically below:

### Example 4

Using the GENE LOGIC® database and the methods described generally in Example 2, the following additional DNA sequences were identified as being overexpressed in colon tumor tissue:

### AA781143/Hs19_{—}11415_{—}28_{—}1_{—}1699a

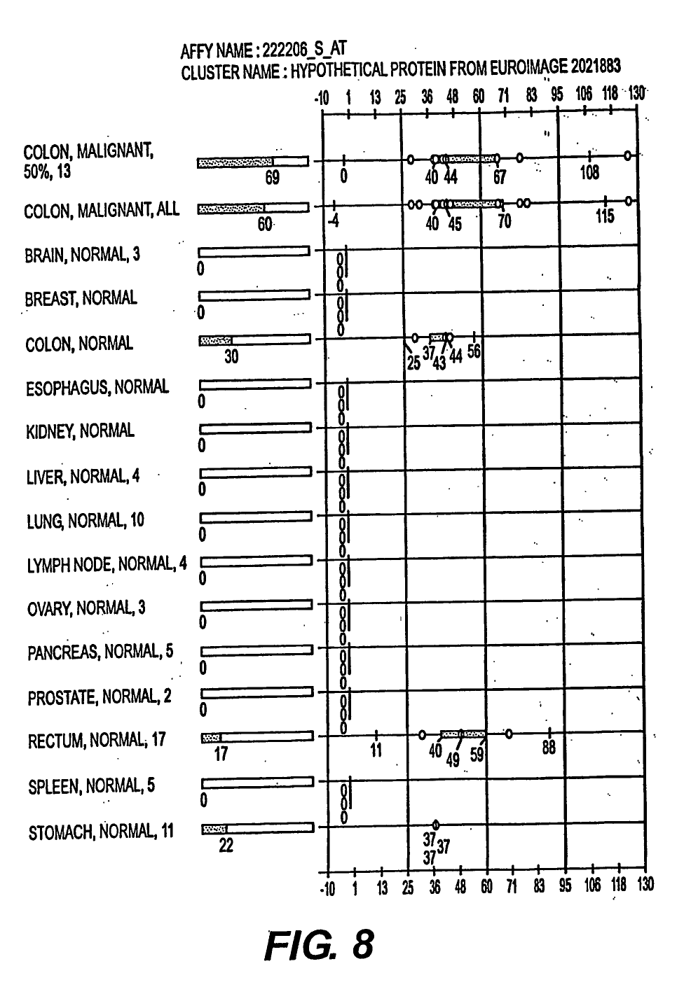

Fragment AA781143 was upregulated 4.16-fold in the colon samples when compared to mixed normal tissue. E-Northern analysis of this fragment demonstrates that it is expressed in 69% of the colon tumors with greater than 50% malignant cells and shows little or no expression in normal tissues. See FIG. 8.

AA781143 Nucleotide Sequence

(SEQ ID NO:36)

TTGTCTTCTACGACCAGCTGAAGCAAGTGATGAATGCGTACAGAGTCAAG
CCGGCCGTCTTTGACCTGCTCCTGGCTGTTGGCATTGCTGCCTACCTCGG
CATGGCCTACGTGGCTGTCCAGGTGAGCAGTGCCCAGGCTCAGCACTTCA
GCCTCCTCTACAAGACCGTCCAGAGGCTGCTCGTGAAGGCCAAGACACAG
TGACACAGCCACCCCCACAGCCGGAGCCCCCGCCGCTCCACAGTCCCTGG
GGCCGAGCACGAGTTGGNAGGGGACCCTCTTCTCCCGTCNTGCCNTCGGG
TTGCCCGCCTCCTCCAGAGACTTNNCAAGGGCCCATCACCACTGGCCTCT
GGGCACTTGTGCTGAGACTCTGGGACCCAGGCAGCTGCCACCTTGTCACC
ATGAGAGAATTTGGGGAGTGCTTGCATGCTAGCCAGCAGGCTCCTGTCTG
GGTGCCACGGGGCCAGCATTTTGGAGGGAGCTTCCTTCCTTCCTTCCTGG
ACAGGTCGTCATGATGGATGCACTGACTGACCGTCTGGGGCTCAGGCTGG
TGTGGGATGCAGCCGGCCG

The GENE LOGIC® database calls this protein “hypothetical protein from EUROIMAGE 2021883.”

EUROLMAGE 2021883 Nucleotide Sequence

(SEQ ID NO:37)

CCAGAGTTTGTCTTCTACGACCAGCTGAAGCAAGTGATGAATGCGTACAG
AGTCAAGCCGGCCGTCTTTGACCTGCTCCTGGCTGTTGGCATTGCTGCCT
ACCTCGGCATGGCCTACGTGGCTGTCCAGCACTTCAGCCTCCTCTACAAG
ACCGTCCAGAGGCTGCTCGTGAAGGCCCAGACACAGTGACACAGCCACCC
CCACAGCCGGAGCCCCCGCCGCTCCACAGTCCCTGGGGCCdAGCACGAGT
GAGTGGACACTGCCCCGCCGCGGGCGGCCCTGCAGGGACAGGGGCCCTCT
CCCTCCCCGGCGGTGGTTGGAACACTGAATTACAGAGCTTTTTTCTGTTG
CTCTCCGAGACTGGGGGGGGATTGTTTCTTCTTTTCCTTGTCTTTGAACT
TCCTTGGAGGAGAGCTTGGGAGACGTCCCGGGGCCAGGCTACGGACTTGC
GGACGAGCCCCCCAGTCCTGGGAGCCGGCCGCCCTCGGTCTGGTGTAAGC
ACACATGCACGATTAAAGAGGAGACGCCGGGACCCCCTGCCCGATCGCGC
GCGGCCTCCGCCCACCGCCTCCTGCCGCAAGGGGCCTGGACTGCAGGCCT
GACCTGCTCCCTGCTCCGTGTCTGTCCTAGGACGTCCCCTCCCGCTCCCC
GATGGTGGCGTGGACATGGTTATTTATCTCTGCTCCTTCTTGCCTGGAGG
AGGGCAGTGCCAGCCCTGGGGTTCTGGGATTCCAGCCCTCCTGGAGCCTT
TTGTTCCCCATGTGGTCTCAGTGACCCGTCCCCCTGACAGTGGGCTCGGG
GAGCTGCATCACCCAGCCTTCCCCTTCTCCGACTGCAGGGTCTGATGTCA
TCATTGACAGCCTTTGCTTCGTGGGGGCCTGGCAGGGCCCCTGCCTCCCC
GACCCCCGACCCACTGCAAATCCCCGTTCCCCTGCACTCCTCTTCTCCCA
GCCCATCCCTCCGGCCCCTGTGCCTCTGCGGCCCCAGCCCAGCTCCCAGG
GCCGTCACCTGCTTGGCCCTGGCCCAGCTCCCTGCCCTGAGTCCTGAGCC
AGTGCCTGGTGTTTCCTGGGCTCGGTACTGGGCCCCCAGGCCATCCAGGC
TTTGCCACGGCCAGTTGGTCCTCCCTGGGGAACTGGGTGCGGGTGGAGTA
CTGGGAGGCAGGAGGTGGCCCGGGGAGGCCTTGTGGCTCCTCCCCTCGCT
CCTCGCCCTGGGCCTCAGCTTCCTCATCAATAGAAAGGATGTGTTCGGGG
TGGGGGCGTCAGGTGAGAACGTTTGCTGGGAAGGAGAGGACTTGGGGCAT
GGCCTCTGGGGCCACCCTTCCTGGAACTCAGAGAGGAAGGTCCGGGCCCT
CGGGAAGCCTTGGACAGAACCCTCCACCCCGCAGACCAGGCGTCGTGTGT
GTGTGGGAGAGAAGGAGGCCCGTGTTGAGCTCAGGGAGACCCCGGTGTGT
CCGTTCTTAGCAATATAACCTACCCAGTGCGTGCCGAGCAGGCTTGGTGG
GGAAGGGACTTGAGCTGGGCAAGTCCTGGCCTGGCACCCGCAGCCGTCTC
CCTTCCGTGGCCCAGGGAGGTGTTTGCTGTCCGAAGGACCTGGGCCGGCC
CATGGGAGCCTGGGGTTCTGTCCAGATAGGACCAGGGGGTCTCACTTTGG
CCACCAGTTCTTCGGCCAGCACCTCTGCCCTCCAGAACCTGCAGCCTGGA
GGGGTGAGGGGACAACCACCCCTCTTTCCTCCAGGTTGGCAGGGGACCCT
CTTCTCCCGTCTGCCCTGCGGGTTGCCCGCCTCCTCCAGAGACTTGCCCA
AGGGCCCATCACCACTGGCCTCTGGGCACTTGTGCTGAGACTCTGGGACC
CAGGCAGCTGCCACCTTGTCACCATGAGAGAATTTGGGGAGTGCTTGCAT
GCTAGCCAGCAGGCTCCTGTCTGGGTGCCACGGGGCCAGCATTTTGGAGG
GAGCTTCCTTCCTTCCTTCCTGGACAGGTCGTCATGATGGATGCACTGAC
TGACCGTCTGGGGCTCAGGCTGGTGTGGGATGCAGCCGGCCGATGAGAAA
ATAAAGCCATATTGAATGAT

EUROIMAGE 2021883 Amino Acid Sequence

(SEQ ID NO:38)

PEFVFYDQLKQVMNAYRVKPAVFDLLLAVGIAAYLGMAYVAVQHFSLLYK
TVQRLLVKAKTQ

The protein set forth above contains one TM (transmembrane domain) by SMART, SOSUI, and TmPred prediction programs. However, the BLAST database and EST sequences suggest that the following alternative nucleotide and protein sequences correspond to AA781143:

Hs19_11415_28_1_1699.a Nucleotide Sequence

(SEQ ID NO:39)

gcaaggtcacgtcctgtccccacctttcgcccctcaccctagctccccca
acgccaaagacaaggttaagaaagtgatatcgcgaaatagttttttaaag
cattttattgcattttatgacttggagtttatgtgaaacctcaacggtat
tagccgaacagcctgccgcaccttccgggagttccagagtgggcctacaa
ctcccacagggctccgcgagcgccggacggacggactacaattcccgaca
ggcagcgcggctggcggggcggttcgccgcggtgcccacaggacctcagg
gcgagtgcgggctgccccgcgcggcgcccgcaggaccccggcggctaccc
atgccgaggtgagtccgcgggagccgccgccgccgccgtcccgtcccagc
tgccgccccgcgcggccccgccgccggccaggATGCTGGAGGAAGCGGGC
GAGGTGCTGGAGAACATGCTGAAGGCGTCTTGTCTGCCGCTCGGCTTCAT
CGTCTTCCTGCCCGCTGTGCTGCTGCTGGTGGCGCCGCCGCTGCCTGCCG
CCGACGCCGCGCACGAGTTCACCGTGTACCGCATGCAGCAGTACGACCTG
CAGGGCCAGCCCTACGGCACACGGAATGCAGTGCTGAACACGGAGGCGCG
CACGATGGCGGCGGAGGTGCTGAGCCGCCGCTGCGTGCTCATGCGGCTAC
TGGACTTCTCCTACGAGCAGTACCAGAAGGCCCTGCGGCAGTCGGCGGGC
GCCGTGGTCATCATCCTGCCCAGGGCCATGGCCGCCGTGCCCCAGGACGT
CGTCCGGCAATTCATGGAGATCGAGCCGGAGATGCTGGCCATGGAGACCG
CCGTCCCCGTGTACTTTGCCGTGGAGGACGAGGCCCTGCTGTCTATCTAC
AAGCAGACCCAGGCTGCCTCCGCCTCCCAGGGCTCCGCCTCTGCTGCTGA
AGTACTGCTGCGCACGGCCACTGCCAACGGCTTCCAGATGGTCACCAGCG
GGGTACAGAGCAAGGCCGTGAGTGACTGGCTGATTGCCAGCGTGGAGGGG
CGGCTGACGGGGCTGGGCGGAGAGGACCTTCCCACCATCGTCATCGTGGC
CCACTACGACGCCTTTGGAGTGGCCCCCTGGCTGTCGCTGGGCGCGGACT
CCAACGGGAGCGGCGTCTCTGTGCTGCTGGAGCTGGCACGCCTCTTCTCC
CGGCTCTACACCTACAAGCGCACGCACGCCGCCTACAACCTCCTGTTCTT
TGCGTCTGGAGGAGGCAAGTTTAACTACCAGGGAACCAAGCGCTGGCTGG
AAGACAACCTGGACCACACAGACTCCAGCCTGCTTCAGGACAATGTGGCC
TTCGTGCTGTGCCTGGACACCGTGGGCCGGGGCAGCAGCCTGCACCTGCA
CGTGTCCAAGCCGCCTCGGGAGGGCACCCTGCAGCACGCCTTCCTGCGGG
AGCTGGAGACGGTGGCCGCGCACCAGTTCCCTGAGGTACGGTTCTCCATG
GTGCACAAGCGGATCAACCTGGCGGAGGACGTGCTGGCCTGGGAGCACGA
GCGCTTCGCCATCCGCCGACTGCCCGCCTTCACGCTGTCCCACCTGGAGA
GCCACCGTGACGGCCAGCGCAGCAGCATCATGGACGTGCGGTCCCGGGTG
GATTCTAAGACCCTGACCCGTAACACGAGGATCATTGCAGAGGCCCTGAG
TCGAGTCATCTACAACCTGACAGAGAAGGGGACACCCCCAGACATGCCGG
TGTTCACAGAGCAGATGCAGATCCAGCAGGAGCAGCTGGACTCGGTGATG
GACTGGCTCACCAACCAGCCGCGGGCCGCGCAGCTGGTGGACAAGGACAG
CACCTTCCTCAGCACGCTGGAGCACCACCTGAGCCGCTACCTGAAGGACG
TGAAGCAGCACCACGTCAAGGCTGACAAGCGGGACCCAGAGTTTGTCTTC
TACGACCAGCTGAAGCAAGTGATGAATGCGTACAGAGTCAAGCCGGCCGT
CTTTGACCTGCTCCTGGCTGTTGGCATTGCTGCCTACCTCGGCATGGCCT
ACGTGGCTGTCCAGCACTTCAGCCTCCTCTACAAGACCGTCCAGAGGCTG
CTCGTGAAGGCCAAGACACAGTGAcacagccacccccacagccggagccc
ccgccgctccacagtccctggggccgagcacgagtgagtggacactgccc
cgccgcgggcggccctgcagggacaggggccctctccctccccggcggtg
gttggaacactgaattacagagcttttttctgttgctctccgagactggg
gggggattgtttcttcttttccttgtctttgaacttccttggaggagagc
ttgggagacgtcccggggccaggctacggacttgcggacgagccccccag
tcctgggagccggccgccctcggtctggtgtaagcacacatgcacgatta
aagaggagacgccgggaccccctgcccgatcgcgcgcggcctccgcccac
cgcctcctgccgcaaggggcctggactgcaggcctgacctgctccctgct
ccgtgtctgtcctaggacgtcccctcccgctccccgatggtggcgtggac
atggttatttatctctgctccttcttgcctggaggagggcagtgccagcc
ctggggttctgggattccagccctcctggagccttttgttccccatgtgg
tctcagtgacccgtccccctgacagtgggctcggggagctgcatcaccca
gccttccccttctccgactgcagggtctgatgtcatcattgacagccttt
gcttcgtgggggcctggcagggcccctgcctccccgacccccgacccact
gcaaatccccgttcccctgcactcctcttctcccagcccatccctccggc
ccctgtgcctctgcggccccagcccagctcccagggccgtcacctgcttg
gccctggcccagctccctgccctgagtcctgagccagtgcctggtgtttc
ctgggctcggtactgggcccccaggccatccaggctttgccacggccagt
tggtcctccctggggaactgggtgcgggtggagtactgggaggcaggagg
tggcccggggaggccttgtggctcctcccctcgctcctcgccctgggcct
cagcttcctcatcaatagaaaggatgtgttcggggtgggggcgtcaggtg
agaacgtttgctgggaaggagaggacttggggcatggcctctggggccac
ccttcctggaactcagagaggaaggtccgggccctcgggaagccttggac
agaaccctccaccccgcagaccaggcgtcgtgtgtgtgtgggagagaagg
aggcccgtgttgagctcagggagaccccggtgtgtccgttctttagcaat
ataacctacccagtgcgtgccgagcaggcttggtggggaagggacttgag
ctgggcaagtcctggcctggcacccgcagccgtctcccttccgtggccca
gggaggtgtttgctgtccgaaggacctgggccggcccatgggagcctggg
gttctgtccagataggaccagggggtctcactttggccaccagttcttcg
gccagcacctctgccctccagaacctgcagcctggaggggtgaggggaca
accacccctctttcctccaggttggcaggggaccctcttctcccgtctgc
cctgcgggttgcccgcctcctccagagacttgcccaagggcccatcacca
ctggcctctgggcacttgtgctgagactctgggacccaggcagctgccac
cttgtcaccatgagagaatttggggagtgcttgcatgctagccagcaggc
tcctgtctgggtgccacggggccagcattttggagggagcttccttcctt
ccttcctggacaggtcgtcatgatggatgcactgactgaccgtctggggc
tcaggctggtgtgggatgcagccggccgatgagaaaataaagccatattg
aatgatcg

Hs19_11415_28_1_1699.a Amino Acid Sequence

(SEQ ID NO:40)

MLEEAGEVLENMLKASCLPLGFIVFLPAVLLLVAPPLPAADAAHEFTVYR
MQQYDLQGQPYGTRNAVLNTEARTMAAEVLSRRCVLMRLLDFSYEQYQKA
LRQSAGAVVIILPRAMAAVPQDVVRQFMEIEPEMLAMETAVPVYFAVEDE
ALLSIYKQTQAASASQGSASAAEVLLRTATANGFQMVTSGVQSKAVSDWL
IASVEGRLTGLGGEDLPTIVIVAHYDAFGVAPWLSLGADSNGSGVSVLLE
LARLFSRLYTYKRTHAAYNLLFFASGGGKFNYQGTKRWLEDNLDHTDSSL
LQDNVAFVLCLDTVGRGSSLHLHVSKPPREGTLQHAFLRELETVAAHQFP
EVRFSMVHKRINLAEDVLAWEHERFAIRRLPAFTLSHLESHRDGQRSSIM
DVRSRVDSKTLTRNTRIIAEALTRVIYNLTEKGTPFDMPVFTEQMQIQQE
QLDSVNDWLTNQPRAAQLVDKDSTFLSTLEHHLSRYLKDVKQHHVKADKR
DPEFVFYDQLKQVMNAYRVKPAVFDLLLAVGIAAYLGMAYVAVQHFSLLY
KTVQRLLVKAKTQ

GenBank also identifies RefSeq Loc56926 as corresponding to AA781143, which nucleotide and protein sequences are set forth below:

RefSeq Loq56926 Nucleotide Sequence

(SEQ ID NO:49)

GGCGAGGTGCTGGAGAACATGCTGAAGGCGTCTTGTCTGCCGCTCGGCTT
CATCGTCTTCCTGCCCGCTGTGCTGCTGCTGGTGGCGCCGCCGCTGCCTG
CCGCCGACGCCGCGCACGAGTTCACCGTGTACCGCATGCAGCAGTACGAC
CTGCAGGGCCAGCCCTACGGCACACGGAATGCAGTGCTGAACACGGAGGC
GCGCACGATGGCGGCGGAGGTGCTGAGCCGCCGCTGCGTGCTCATGCGGG
TACTGGACTTCTCCTACGAGCAGTACCAGAAGGCCCTGCGGCAGTCGGCG
GGCGCCGTGGTCATCATCCTGCCCAGGGCCATGGCCGCCGTGCCCCAGGA
CGTCGTCCGGCAATTCATGGAGATCGAGCCGGAGATGCTGGCCATGGAGA
CCGCCGTCCCCGTGTACTTTGCCGTGGAGGACGAGGCCCTGCTGTCTATC
TACAAGCAGACCCAGGCTGCCTCCGCCTCCCAGGGCTCCGCCTCTGCTGC
TGAAGTACTGCTGCGCACGGCCACTGCCAACGGCTTCCAGATGGTCACCA
GCGGGGTACAGAGCAAGGCCGTGAGTGACTGGCTGATTGCCAGCGTGGAG
GGGCGGCTGACGGGGCTGGGCGGAGAGGACCTTCCCACCATCGTCATCGT
GGCCCACTACGACGCCTTTGGAGTGGCCCCCTGGCTGTCGCTGGGCGCGG
ACTCCAACGGGAGCGGCGTCTCTGTGCTGCTGGAGCTGGCACGCCTCTTC
TCCCGGCTCTACACCTACAAGCGCACGCACGCCGCCTACAACCTCCTGTT
CTTTGCGTCTGGAGGAGGCAAGTTTAACTACCAGGGAACCAAGCGCTGGC
TGGAAGACAACCTGGACCACACAGACTCCAGCCTGCTTCAGGACAATGTG
GCCTTCGTGCTGTGCCTGGACACCGTGGGCCGGGGCAGCAGCCTGCACCT
GCACGTGTCCAAGCCGCCTCGGGAGGGCACCCTGCAGCACGCCTTCCTGC
GGGAGCTGGAGACGGTGGCCGCGCACCAGTTCCCTGAGGTACGGTTCTCC
ATGGTGCACAAGCGGATCAACCTGGCGGAGGACGTGCTGGCCTGGGAGCA
CGAGCGCTTCGCCATCCGCCGACTGCCCGCCTTCACGCTGTCCCACCTGG
AGAGCCACCGTGACGGCCAGCGCAGCAGCATCATGGACGTGCGGTCCCGG
GTGGATTCTAAGACCCTGACCCGTAACACGAGGATCATTGCAGAGGCCCT
GACTCGAGTCATCTACAACCTGAGAGAGAAGGGGACACCCCCAGACATGC
CGGTGTTCACAGAGCAGATGCAGATCCAGCAGGAGCAGCTGGACTCGGTG
ATGGACTGGCTCACCAACCAGCCGCGGGCCGCGCAGCTGGTGGACAAGGA
CAGCACCTTCCTCAGCACGCTGGAGCACCACCTGAGCCGCTACCTGAAGG
ACGTGAAGCAGCACCACGTCAAGGCTGACAAGCGGGACCCAGAGTTTGTC
TTCTACGACCAGCTGAAGCAAGTGATGAATGCGTACAGAGTCAAGCCGGC
CGTCTTTGACCTGCTCCTGGCCGTTGGCATTGCTGCCTACCTCGGCATGG
CCTACGTGGCTGTCCAGCACTTCAGCCTCCTCTACAGGACCGTCCAGAGG
CTGCTCGTGAAGGCCAAGACACAGTGACACAGCCACCCCCACAGCCGGAG
CCCCCGCCGCTCCACAGTCCCTGGGGCCGAGCACGAGTGAGTGGACACTG
CCCCGCCGCGGGCGGCCCTGCAGGGACAGGGGCCCTCTCCCTCCCCGGCG
GTGGTTGGAACACTGAATTACAGAGCTTTTTTCTGTTGCTCTCCGAGACT
GGGGGGGGATTGTTTCTTCTTTTCCTTGTCTTTGAACTTCCTTGGAGGAG
AGCTTGGGAGACGTCCCGGGGCCAGGCTACGGACTTGCGGACGAGCCCCC
CAGTCCTGGGAGCCGGCCGCCCTCGGTCTGGTGTAAGCACACATGCACGA
TTAAAGAGGAGACGCCGGGACCCCCTGCCCGATCGCGCGCGGCCTCCGCC
CACCGCCTCCTGCCGCAAGGGGCCTGGACTGCAGGCCTGACCTGCTCCCT
GCTCCGTGTCTGTCCTAGGACGTCCCCTCCCGCTCCCCGATGGTGGCGTG
GACATGGTTATTTATCTCTGCTCCTTCTTGCCTGGAGGAGGGCAGTGCCA
GCCCTGGGGTTCTGGGATTCCAGCCCTCCTGGAGCCTTTTGTTCCCCATG
TGGTCTCAGTGACCCGTCCCCCTGACAGTGGGCTCGGGGAGCTGCATCAC
CCAGCCTTCCCCTTCTCCGACTGCAGGGTCTGATGTCATCGTTGACAGCC
TTTGCTTCGTGGGGGCCTGGCAGGGCCCCTGCCTCCCCGACCCCCGACCC
ACTGCAAACCCCCGTTCCCCTGCACTCCTCTTCTCCCAGCCCATCCCTCC
GGCCCCTGTGCCTCTGCGGCCCCAGCCCAGCTCCCAGGGCCGTCACCTGC
TTGGCCCTGGCCCAGCTCCCTGCCCTGAGTCCTGAGCCAGTGCCTGGTGT
TTCCTGGGCTCGGTACTGGGCCCCCAGGCCATCCAGGCTTTGCCACGGCC
AGTTGGTCCTCCCTGGGGAACTGGGTGCGGGTGGAGTACTGGGAGGCAGG
AGGTGGCCCGGGGAGGCCTTGTGGCTCCTCCCCTCGCTCCTCGCCCTGGG
CCTCAGCTTCCTCATCAATAGAAAGGATGTGTTCGGGGTGGGGGCGTCAG
GTGAGAACGTTTGCTGGGAAGGAGAGGACTTGGGGCATGGCCTCTGGGGC
CACCCTTCCTGGAACTCAGAGAGGAAGGTCCGGGCCCTCGGGAAGCCTTG
GACAGAACCCTCCACCCCGCAGACCAGGCGTCGTGTGTGTGTGGGAGAGA
AGGAGGCCCGTGTTGAGCTCAGGGAGACCCCGGTGTGTCCGTTCTTTAGC
AATATAACCTACCCAGTGCGTGCCGAGCAGGCTTGGTGGGGAAGGGACTT
GAGCTGGGCAAGTCCTGGCCTGGCACCCGCAGCCGTCTCCCTTCCGTGGC
CCAGGGAGGTGTTTGCTGTCCGAAGGACCTGGGCCGGCCCATGGGAGCCT
GGGGTTCTGTCCAGATAGGACCAGGGGGTCTCACTTTGGCCACCAGTTCT
TCGGCCAGCACCTCTGCCCTCCAGAACCTGCAGCCTGGAGGGGTGAGGGG
ACAACCACCCCTCTTTCCTCCAGGTTGGCAGGGGACCCTCTTCTCCCGTC
TGCCCTGTGGGTTGCCCGCCTCCTCCAGAGACTTGCCCAAGGGCCCATCA
CCACTGGCCTCTGGGCACTTGTGCTGAGACTCTGGGACCCAGGCAGCTGC
CACCTTGTCACCATGAGAGAATTTGGGGAGTGCTTGCATGCTAGCCAGCA
GGCTCCTGTCTGGGTGCCACGGGGCCAGCATTTTGGAGGGAGCTTCCTTC
CTTCCTTCCTGGACAGGTCGTCAGGATGGATGCACTGACTGACCGTCTGG
GGCTCAGGCTGGTGTGGGATGCAGCCGGCCGATGAGAAAATAAAGCCATA
TTGAATGATAAAAAAAAAAAAAAAAAA

RefSeq Loq56926 Amino Acid Sequence

(SEQ ID NO:50)

MLKASCLPLGFIVFLPAVLLLVAPPLPAADAAHEFTVYRMQQYDLQGQPY
GTRNAVLNTEARTMAAEVLSRRCVLMRLLDFSYEQYQKALRQSAGAVVII
LPRAMAAVPQDVVRQFMEIEPEMLAMETAVPVYFAVEDEALLSIYKQTQA
ASASQGSASAAEVLLRTATANGFQMVTSGVQSKAVSDWLIASVEGRLTGL
GGEDLPTIVIVAHYDAFGVAPWLSLGADSNGSGVSVLLELARLFSRLYTY
KRTHAAYNLLFFASGGGKFNYQGTKRWLEDNLDHTDSSLLQDNVAFVLCL
DTVGRGSSLHLHVSKPPREGTLQHAFLRELETVAAHQFPEVRFSMVHKRI
NLAEDVLAWEHERFAIRRLPAFTLSHLESHRDGQRSSIMDVRSRVDSKTL
TRNTRIIAEALTRVIYNLTEKGTPPDMPVFTEQMQIQQEQLDSVMDWLTN
QPRAAQLVDKDSTFLSTLEHHLSRYLKDVKQHHVKADKRDPEFVFYDQLK
QVMNAYRVKPAVFDLLLAVGIAAYLGMAYVAVQHFSLLYRTVQRLLVKAK
TQ

The RefSeq Loq56926 protein has a transmembrane domain as predicted by SOSUI and TmPred. It also has both a signal peptide and a transmembrane domain predicted by SMART, suggesting that this is a type I membrane protein with the majority of the protein being extracellular.

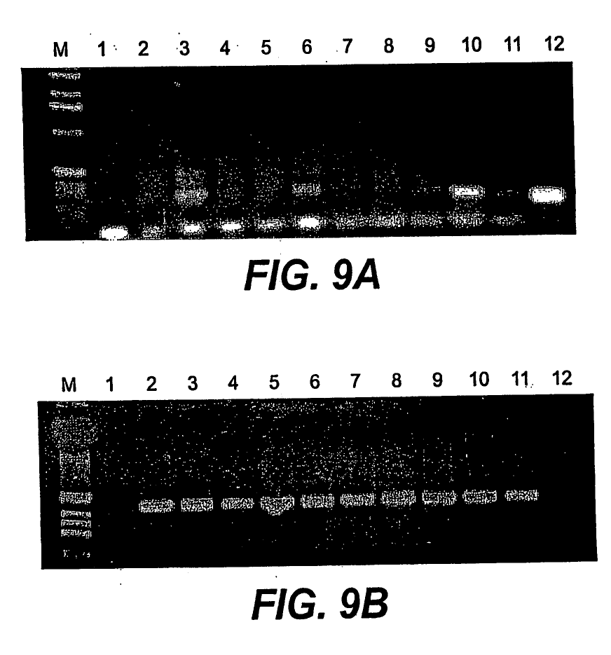

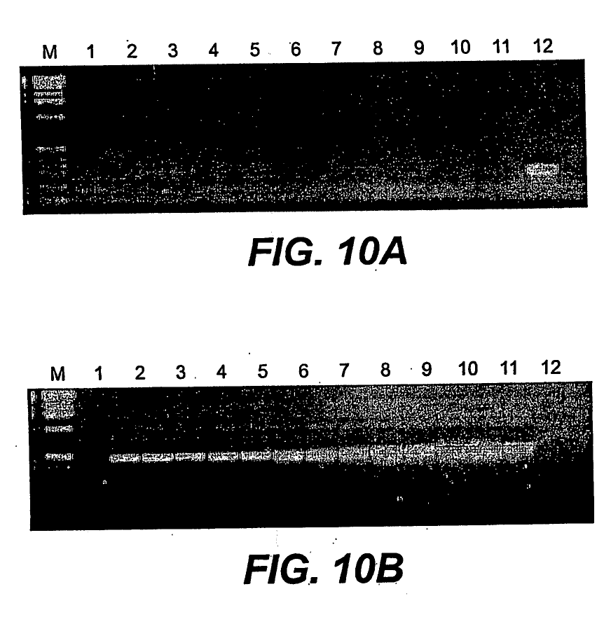

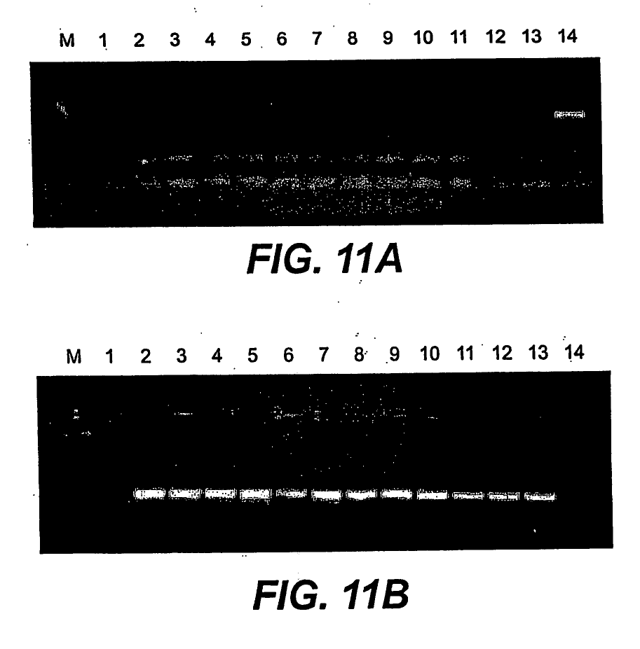

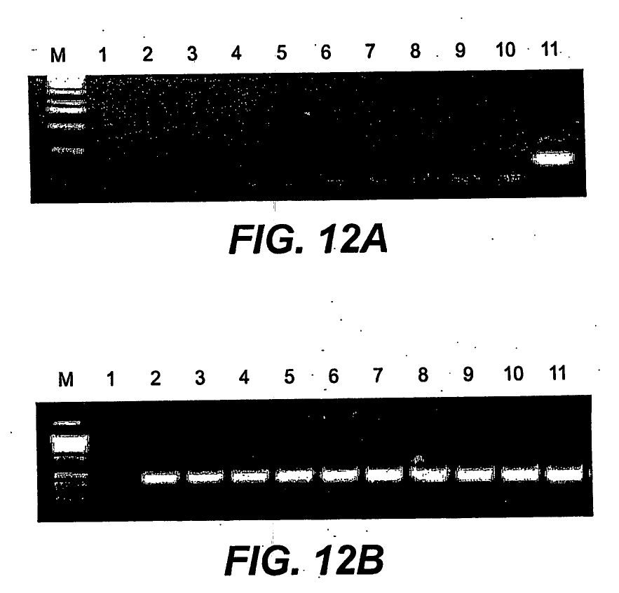

The expression of Loc56926 in normal and malignant human tissues was further investigated by PCR experiments using commercially available human cDNA panels and cDNA samples prepared in-house from human tissues and cell lines. See FIGS. 9A-9B, 10A-10B, 11A-11B, and 12A-12B. Expression of Glyceraldehyde 3-phosphate dehydrogenase (GAPDH) was measured in these experiments as a control for cDNA integrity. GAPDH is a housekeeping gene expressed abundantly in all human tissues. The following primers were used to amplify a 482 base pair product of the GAPDH gene:

| | |
|---|---|
| 5′′ACCACAGTCCATGCCATCAC 3′ | (SEQ ID NO:62) |
| 5′ TCCACCACCCTGTTGCTGTA 3′ | (SEQ ID NO:63) |

For expression studies, malignant colon samples were obtained from Analytical Pathology Medical Group and frozen within thirty minutes of surgery. The HCT116 colon cancer cell line was obtained from American Type Culture Collection (ATCC of Manassas, Va.). RNA was extracted from the samples using RNEASY® Maxi Kit (Qiagen #75162) or from fresh HCT116 cells using the RNEASY® Mini kit (Qiagen, #74104) according to the manufacture's instructions and reverse transcribed into cDNA using SUPERSCRIPT® II Kit (Invitrogen # 12371-019). The positive control for Loc56926 IMAGE clone 4428206 was obtained from the ATCC. Primers used to amplify a 283 base pair product of Loc56926 were:

| | |
|---|---|
| 5′ AATGCAGTGCTGAACACGGAG 3′ | (SEQ ID NO:64) |
| 5′ TCTGCTTGTAGATAGACAGCAGG 3′ | (SEQ ID NO:65) |

### AW779536

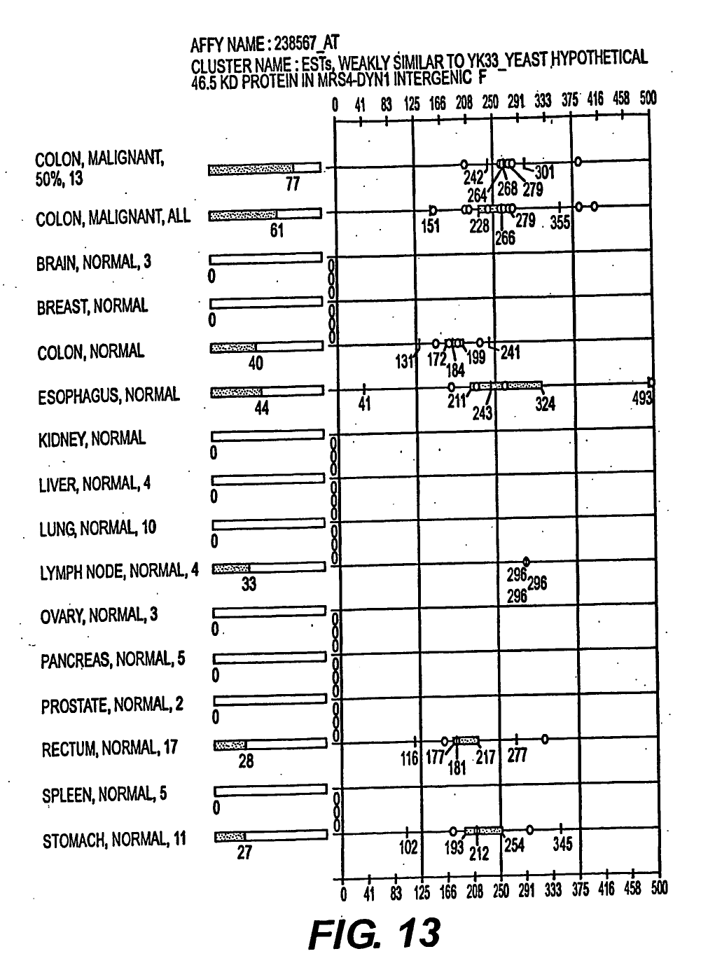

In a comparison of malignant colon samples containing greater than 50% malignant cells in the sample against mixed normal tissues, fragment AW779536 was upregulated 3.7 fold. E-Northern analysis shown in FIG. 13 demonstrates that the fragment is expressed in 77% of the tumors and poorly expressed in normal tissue.

AW779536 Nucleotide Sequence

(SEQ ID NO:41)

TTCTTCCTGTGTTACAATTACCCTGTTTCTGATTACTACAGCCCAACCCG
GGCGGACACCACCACCATTCTGGCTGCCGGGGCTGGAGTGACCATAGGAT
TCTGGATCAACCATTTCTTCCAGCTTGTATCCAAGCCCGCTGAATCTCTC
CCTGTTATTCAGAACATCCCACCGNTCACCACCTACATGTTAGNTTTGGG
TCTGACCAAATTTGCAGTGGGAATTGTGTTGATCCTCTTGGTTCGTCAGC
TTGTACAAAATCTCTCACTGCAAGTATTATACTCATGGTTCNAGGTNGGT
CNCCAGGAACAAGGAGGCCAGGCGGAGACTGGAGATTGAAGTGCCTTACA
AGTTTGTTACCTACACATCTGTTGGCATCTGCGCTACAACCTTTGTGCCG
ATGCTTCACAGGTTTCTGGGATTACCCTGAGTCTCAAACAGTTGGAAACT
AGCCCACTGGACATGAAAGCCAAGACATAGGAAAGTTATTGGTAGGCAAA
TCTTGACAACTTATTTTTCTTTAACAACAACAAAAAGTCATACGGCTGTC
TTGCTACT

BLAST searching with this sequence revealed a hypothetical protein predicted by Acembly, Ensembl and Fgenesh++, Hs2_{—}5283_{—}28_{—}1_{—}1143.b with the following nucleotide sequence:

Hs2_5283_28_1_1143.b Nucleotide Sequence

(SEQ ID NO:42)

GCTTATGTACAGAAGTACGTCGTGAAGAATTATTTCTACTATTACCTATT
CCAATTTTCAGCTGCTTTGGGCCAAGAAGTGTTCTACATCACGTTTCTTC
Cattcactcactggaatattgacccttatttatccagaagattgatcatc
atatgggttttggtgatgtatattggccaagtggccaaggatgtcttgaa
gtggccccgtccctcctcccctccagttgtaaaactggaaaagagactga
tcgctgaatatggaatgccatccacccacgccatggcggccactgccatt
gccttcaccctccttatctctactatggacagataccagtatccatttgt
gttgggactggtgatggccgtggtgttttccaccttggtgtgtctcagca
ggctctacactgggatgcatacggtcctggatgtgctgggtggcgtcctg
atcaccgcactcctcatcgtcctcacctaccctgcctggaccttcatcga
ctgcctggactcggccagccccctcttccccgtgtgtgtcatagttgtgc
cattcttcctgtgttacaattaccctgtttctgattactacagcccaacc
cgggcggacaccaccaccattctggctgccggggctggagtgaccatagg
attctggatcaaccatttcttccagcttgtatccaagcccgctgaatctc
tccctgttattcagaacatcccaccactcaccacctacatgttagttttg
ggtctgaccaaatttgcagtgggaattgtgttgatcctcttggttcgtca
gcttgtacaaaatctctcactgcaagtattatactcatggttcaaggtgg
tcaccaggaacaaggaggccaggcggagactggagattgaagtgccttac
aagtttgttacctacacatctgttggcatctgcgctacaacctttgtgcc
gatgcttcacaggtttctgggattaccctgagtctcaaacagttggaaac
tagcccactggacatgaaagccaagacataggaaagttattggtaggcaa
atcttgacaacttatttttctttaacaacaacaaaaagtcatacggctgt
cttgctactaccagataaatgatgctgctgtgtgaaaggaagaactgtct
catagcggtcattggtcgtccgtggtggttggttgtgctacagttgaacc
caggctaaagaccataatccggatctttaaaggcacacaccgcgcccccc
ccccccccgcccggcccctgctcctctcgctgttgcacgggctttggatc
tagtcatgggctggcaggaattgtggcctggcttaggaatagctatgagc
cccactgggttctggagagccagtagagatggggtgatctgggaggctgg
aggtagagcctttcttttccgttacaaccttgcctagcatggagttaact
gtgcctggttgggtggtaagatcactctgaaagaaagctcactgtgaaga
gatgaaaggtggaggcagagctgtgaggtcatggggaaaagcctgctttc
cttataagtcctgctgttcatgttggaataaggatctgctcttccttgtt
tccatgcattttgcaggattccaggtaccattaccacactcttctgaccc
atgaaaccaactggctgctcacacatcaccaaacaggttgggggttagcc
ttcagcacaggtggatacatctgggattcactgagattcctgccctctcc
tgcttcctagtggtttgggacaggccctctgcccatcgtcagcagttttt
tgctttcatacaaacctggaaggcactggcatctgcctaggaaagtggat
ctgtgaagaacagatgaactcaatcctttctggagtctgacaaagaaggg
ataggcttccttgacattgcctgtcctgacaaggcctccctgacattact
cctccaatttcacagttaccttctgtaaatctattttctcatctactgaa
tagaatcaggcgccctttttgtcttcccacctcttatctcttggcaattt
taaggggaattaatgcaagaacaactttagtgtctcttgggaaaacaagc
caaccaaatacaaaacccattaagcctactagggtgagtcctcttaacat
gggaaggcgatgattatgcaaacaccggagttccctcctcttcagttcct
aagaataaagaacaggtatcaagaactttctttaaagttagtgtaactat
agttaacaaagtatccattgaagtttagtgcctgtaggactgagccagtg
ctttatcaacccaacacatcatcaccatgtgcatactctagaaaaaaaaa
tagcttccttaaaagttacagaggctcttaacgtgttaaaaccgaaaaat
cacatttttcttgatttcaaatatgttctacggccttactgttgggatga
tatttagtatgtaacttagcattccaatttctcaagaatttttaggccgg
gtgcggtggctcatgcctgtaatcccagcactttgggaggccgaggtggg
cggaccacgaggtcaggagatcgagaccatcctggctaacacggtacccc
gtctctactgaaaatacaaaaaaattagccggacgtggtggagggcgcct
gtagtcccagctactcaggaggctgaggcaggagaatggcgtgaacccgg
tgagcggagcttgcagtgagccgagattgcgccactgcactccagcctgg
gcgacagagcgagactctctcaaaaaaaaaaaaaaagaatttttagcaaa
acatcctgtttttacttaaaattcttctcatatttattatagttagaagg
caaagatcaagatgacctgccgtttgactgcttttacatcaaactctgcc
cagtatttgcagcacaactcaggggaagggccttagcttacaggtactcc
cagccttcatctgcccctgcagagcagtggctgtcagccggatgcggcac
ttttctgtattttcatccacacagctgcccagccagagttcgcaacactg
gatatttacaccaaataattgtggttgacttgtctgaagccagctgacaa
aaggatcagcttttcccacttgtattttttaaaaagagggattgtgatca
ttgtcacagagtgggtgctggcctctcatatatatgatatatatatatca
ttttatatatatatatatatcatatacataatttttactgctgtctctag
ttttaagtcccaacaataggaaggccgatcagctatattgatatatttaa
ggctgtacttaactaatttgggctgaggatgaatatatcagccacagcac
attaaagaatgagccaaggatttgtcatggttggtcactttttaaagtat
ttgattactgcaactggagaatgaaaagtgtatattggtgacgccaacct
cagtttctgagcactcctgctctgtggtgagaatcagacaaaaattcatc
ggggtgaaaaaggcattacctgattcacacccttgtcttgctagccctct
tccattcatttctcacacagcactttgctctgttaaatcctctctctgtc
tcagaccattgcttgccccttcaaagggtatggttcaggctcctttcaag
acatttggagtttctctctggggaaagagagccccctactggtttggctt
cagtctaggtccaccatccctctcgatctggcatcttggagattaattta
aaaggcaagctcaccacaatgtaagcctatggtctggccaaccttgcttt
tgggaactgtgacaccaaagcccccaggactatctgcctctccaggagcc
agatagaatgacatgcctttttcctaattgtccacattccacccccaacc
cactgccactgtgggccaagccatccatcttgcaatcttcatctaaaaca
gctctcatttcatgccagttttgctcaaacctgcaccgtcacaagatatt
cagaagatgaaaacgtagaagacacccctgaattaaaaacacttacatag
cagtggctggaattactccaaaacgtgcccagtgatcgcactgtaacatg
ggattttctcacccaaataggcaactcatgcttcctgagtgtaatcaaag
catgtggtgttttggggccatatgcaccaggtttctattttagaaacctt
cagctgtcttgcttatgtactgtatgtaaatttattctttttaaaaatca
cttttatttgattttgacttattaaatgctttaaaagccag

The amino acid sequence of Hs2_{—}5283_{—}281_{—}1_{—}143.b is set forth below:

Hs2_5283_28_1_1143.b Amino Acid Sequence

(SEQ ID NO:43)

AYVQKYVVKNYFYYYLFQFSAALGQEVFYITFLPFTHWNIDPYLSRRLII
IWVLVMYIGQVAKDVLKWPRPSSPPVVKLEKRLIAEYGMPSTHAMAATAI
AFTLLISTMDRYQYPFVLGLVMAVVFSTLVCLSRLYTGMHTVLDVLGGVL
ITALLIVLTYPAWTFIDCLDSASPLFPVCVIVVPFFLCYNYPVSDYYSPT
RADTTTILAAGAGVTIGFWINHFFQLVSKPAESLPVIQNIPPLTTYMLVL
GLTKFAVGIVLILLVRQLVQNLSLQVLYSWFKWTRNKEARRRLEIEVPY
KFVTYTSVGICATTFVPMLHRFLGLP

This amino acid sequence is predicted to contain 9 transmembrane domains by SMART and TmPred and 8 transmembrane domains by SOSUI. By contrast, when analyzed by use of the GENEID™ program, the following gene is identified as being overexpressed in colon tissue:

chr2_2054 Nucleotide Sequence

(SEQ ID NO:44)

ATGGCGGCCACTGCCATTGCCTTCACCCTCCTTATCTCTACTATGGACAG
ATACCAGTATCCATTTGTGTTGGGACTGGTGATGGCCGTGGTGTTTTCCA
CCTTGGTGTGTCTCAGCAGGCTCTACACTGGGATGCATACGGTCCTGGAT
GTGCTGGGTGGCGTCCTGATCACCGCACTCCTCATCGTCCTCACCTACCC
TGCCTGGACCTTCATCGACTGCCTGGACTCGGCCAGCCCCCTCTTCCCCG
TGTGTGTCATAGTTGTGCCATTCTTCCTGTGTTACAATTACCCTGTTTCT
GATTACTACAGCCCAACCCGGGCGGACACCACCACCATTCTGGCTGCCGG
GGCTGGAGTGACCATAGGATTCTGGATCAACCATTTCTTCCAGCTTGTAT
CCAAGCCCGCTGAATCTCTCCCTGTTATTCAGAACATCCCACCACTCACC
ACCTACATGTTAGTTTTGGGTCTGACCAAATTTGCAGTGGGAATTGTGTT
GATCCTCTTGGTTCGTCAGCTTGTACAAAATCTCTCACTGCAAGTATTAT
ACTCATGGTTCAAGGTGGTCACCAGGAACAAGGAGGCCAGGCGGAGACTG
GAGATTGAAGTGCCTTACAAGTTTGTTACCTACACATCTGTTGGCATCTG
CGCTACAACCTTTGTGCCGATGCTTCACAGGTTTCTGGGATTACCCTGA

This gene encodes a protein having the following predicted structure:

chr2_2054 Amino Acid Sequence

(SEQ ID NO: 45)

MAATAIAFTLLISTMDRYQYPFVLGLVMAVVFSTLVCLSRLYTGMHTVLD
VLGGVLITALLIVLTYPAWTFIDCLDSASPLFPVCVIVVPFFLCYNYPVS
DYYSPTRADTTTILAAGAGVTIGFWINHFFQLVSKPAESLPVIQNIPPLT
TYMLVLGLTKFAVGIVLILLVRQLVQNLSLQVLYSWFKVVTRNKEARRRL
EIEVPYKFVTYTSVGICATTFVPMLHRFLGLP*

When this sequence is analyzed by SOSUI and TmPred it is predicted to possess 7 transmembrane domains. By contrast, analyses by SMART suggests that the protein has 5 transmembrane domains and a signal sequence. These analyses also indicate that the protein contains a PFAM domain indicating that the protein contains an acid phosphatase domain.

### AL531683

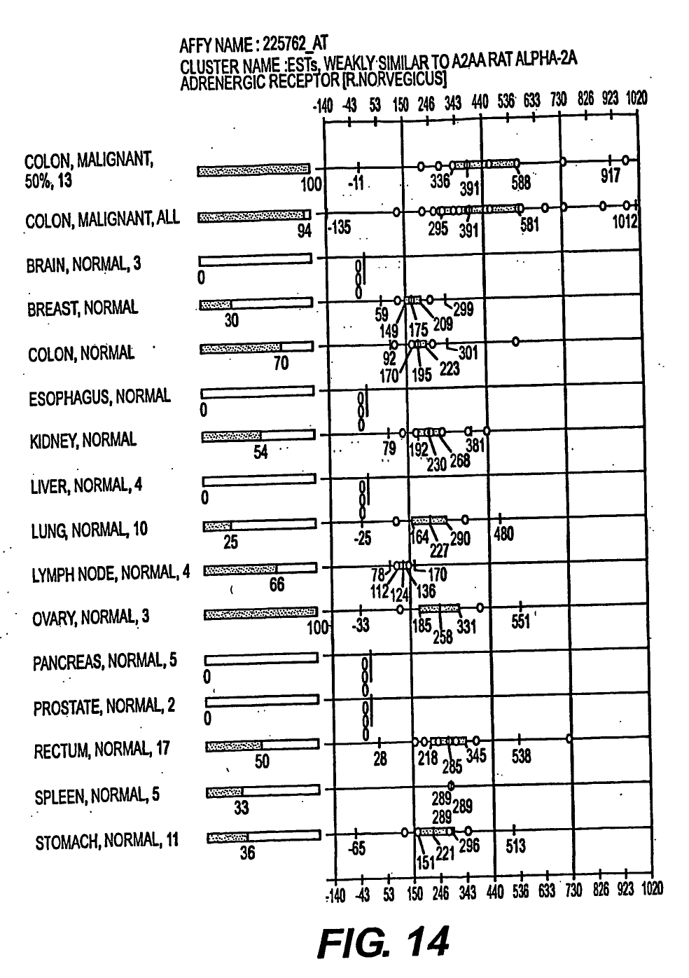

In a comparison of malignant colon samples with greater than 50% malignant cells in the sample against mixed normal tissues, fragment AL531683 was found to be upregulated 3.76-fold. The E-Northern analysis shown in FIG. 14 demonstrates that the fragment is expressed in 100% of the tumors analyzed and poorly expressed in normal tissue.

AL53168 Nucleotide Sequence

(SEQ ID NO:46)

CGCCGGCGGTGCGTGTGGGAAGGCGTGGGGTGCGGACCCCGGCCCGACCT
CNCCGTCCCGCCCGCCGCCTTCTGCGTCGCGGGNGCGGGCCGGCGGGGTC
CTCTGACGCGGCAGACAGNCCCTCGCTGTCGCCTCCAGTGGTTGTCGACT
TGCGGGCGGCCCCCCTCCGCGGCGGTGGGGGTGCCGTCCCGCCGGCCCGT
CGTGCTGCCCTCTCNNGGGGGGTTTGCGCGAGCGTCGGCTCCGCCTGGGC
CCTTGCGGTGCTCCTGGAGCGCTCCGGGTTGTCCCTCAGGTGCCCGAGGC
CGAACGGTGGTGTGTCGTTCCCGCCCCCGGCGCCCCCTCCTCCGGTCGCC
GCCGCGGTGTCCGCGCGTGGGTCCTGAGGGAGCTCGTCGGTGTGGGGTTC
GAGGCGGTTTGAGTGAGACGAGACGAGAC

### AI202201

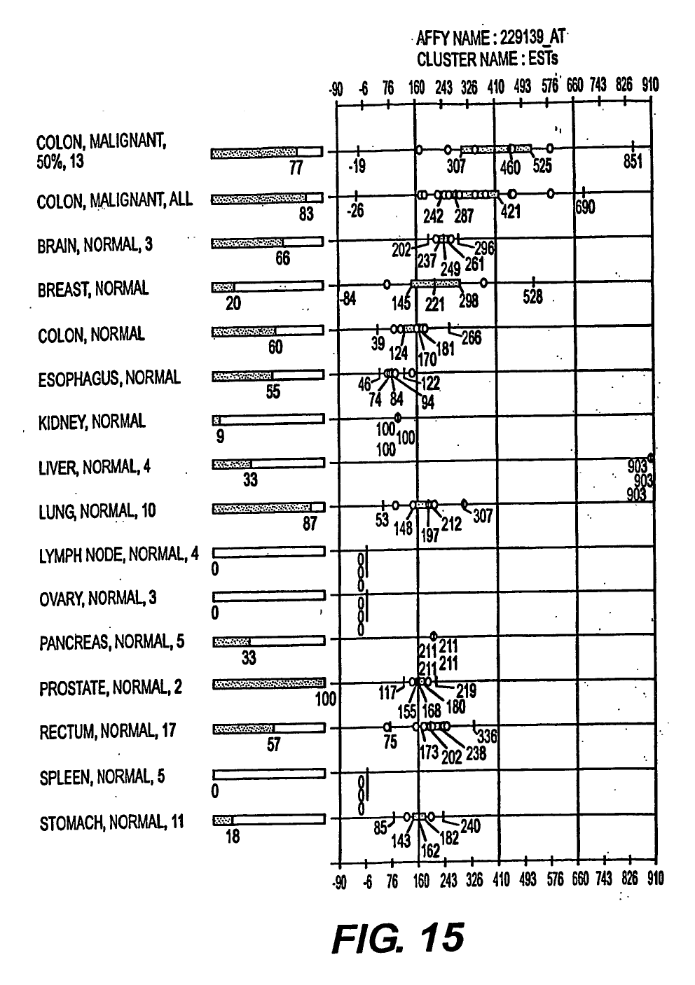

In a comparison of malignant colon samples with greater than 50% malignant cells in the sample against mixed normal tissues, fragment AI202201 was upregulated 3.18-fold. E-Northern analysis shown in FIG. 15 demonstrates that the fragment is expressed in 77% of the tumors and poorly expressed in normal tissue.

AI202201 Nucleotide Sequence

(SEQ ID NO:47)

ACCCTATAGCTCCTTACGCTGGGAAAGCTGGTTTTTTAAAAAAATAATAA
TAAAATATTTAATCTTATTAAGTGTTCATTTAAAATGCGTAATGCTTTGG
AAATAATGGGTAACAGATAGCGAGAGGATATGTTTATAAAGTGAGCATGT
TGGTCCCATTTATAAATATATGTATGATTTATAAGCTTTTTTAAAACAAA
GCTCAAATTGTTGGTATTTTTCTAAAATGTGCACAGCTGTATTTTACATG
AAGGCTCTTTCTAATGGGTTGTTATACTGTACTCAACATTTTGGACAGCA
CATGAAGTCTGCCAATGTACTTAATAAAACATGACTTTGTTTATTTAAAG
TTTCTTGCTGTGAAAAAGAACTCCCTACCTGTGAGTTCCTTTATTTATAA
TTCTTGAAACCAAAATGTATAATGTACAGTTTTCACAACTGTATCTGCTC
TAATA

### AL389942

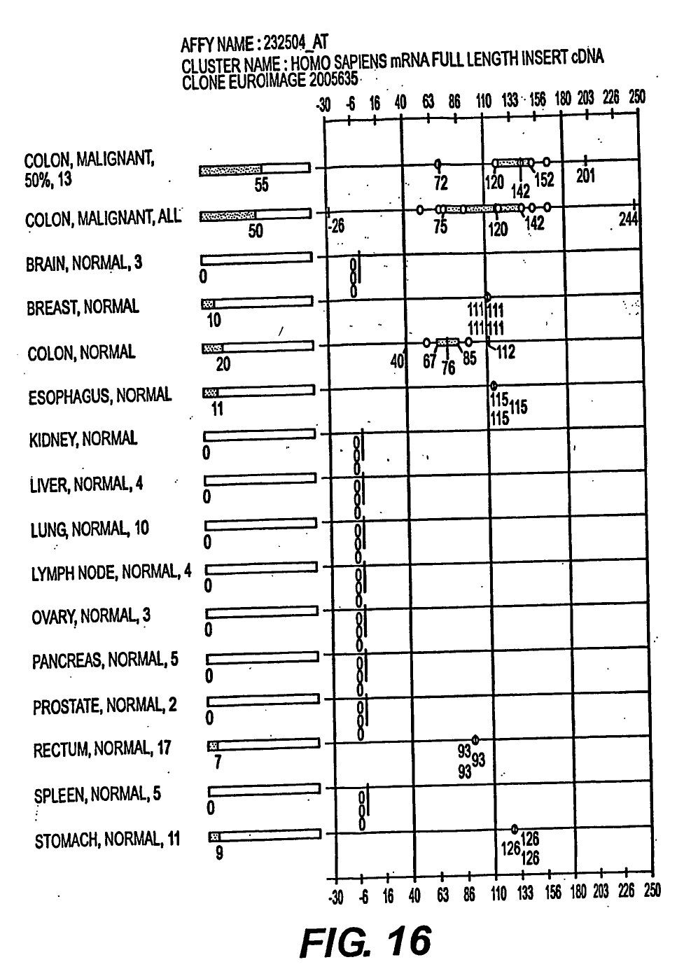

In a comparison of malignant colon samples with greater than 50% malignant cells in the sample against mixed normal tissues, fragment AL389942 was upregulated 3.83-fold. E-Northern analysis shown in FIG. 16 demonstrates that the fragment is expressed in 55% of the tumors and poorly expressed in normal tissue.

AL389942 Nucleotide Sequence

(SEQ ID NO:48)

GAAGCTCCAAATGCTCTGGGTTTCAGCTCCTCTGTGCTGTGGACNCTGAC
TTTGGCTCAGAACTCCGATTTAGTACAAAAGGCTCATTTTTATTTCAGGG
GCACTCTTCCTAAAGCAAACCTAATAAATGAAATATGGAATTCACAGATA
CACACACACATTAAAAAATTAACCTAGTGTATCTGTGAGGAGTAGGCAGA
AATTCNCTGTATAAAAGAATGCTTCATTTCATAGAGAATTTGTGTTAAGA
TTCCATTAGATAGTACATTTCTCAAAGATTTTTGAGGTTGTATTTGCTTT
ACCAAAACTTGGTTTATGTAAGTGGAAAAAGCATGTTGCAAAATAACTTG
GTGTCTATGATTCAGTTTATGTAAAATAATAAATGTATGTAGGAATACGT
GTGTTGAAAGATGTACATCAATTTGCTAACAATGGTTATCTCTGACGTGG
TGGGATTTGAGATGTGTTTTTCTTTTTGGTTGTATTTTTCTCTATTGTTT
GACTTA

### Example 5

### Identification of Gene Upregulated in Colon Cancer

Using the GENE LOGIC® database and the methods described generally in Example 2, the following additional DNA sequences were identified as being overexpressed in colon tumor tissue:

DNA fragment NM_{—}021246 is 5-fold upregulated as shown by hybridization in the malignant colon when compared with mixed normal samples, greater than 3-fold upregulated compared with normal kidney, liver and lung, and greater than 2-fold upregulated in all other tissues.

NM_021246 Nucleotide Sequence

(SEQ ID NO:56)

AACCGAATGCGGTGCTACAACTGTGGTGGAAGCCCCAGCAGTTCTTGCAA
AGAGGCCGTGACCACCTGTGGCGAGGGCAGACCCCAGCCAGGCCTGGAAC
AGATCAAGCTACCTGGAAACCCCCCAGTGACCTTGATTCACCAACATCCA
GCCTGCGTCGCAGCCCATCATTGCAATCAAGTGGAGACAGAGTCGGTGGG
AGACGTGACTTATCCAGCCCACAGGGACTGCTACCTGGGAGACCTGTGCA
ACAGCGCCGTGGCAAGCCATGTGGCCCCTGCAGGCATTTTGGCTGCAGCA
GCTACCGCCCTGACCTGTCTCTTGCCAGGACTGTGGAGCGGATAGGGGGA
GTAGGAGTAGAGAAGGGAACAAGGGAGCAAGGGAACAAGGGACATCTGAA
CATCT

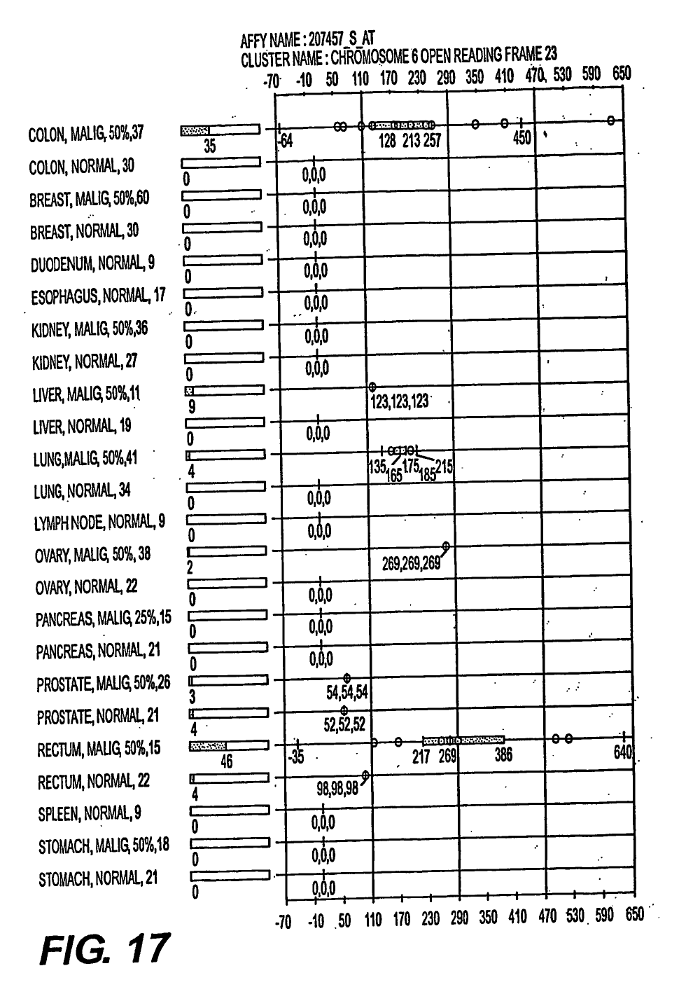

The E-northern results in FIG. 17 indicate that this fragment is upregulated in colon and rectal malignancies. Accordingly, this gene can be targeted for the treatment of colon or rectal cancer. A search of commercial databases reveals that NM_{—}021246 is apparently part the Ly6G6D gene set forth below:

Ly6G6D mRNA Sequence

(SEQ ID NO:57)

cccatggcagtcttattcctcctcctgttcctatgtggaactccccaggc
tgcagacaacatgcaggccatctatgtggccttgggggaggcagtagagc
tgccatgtccctcaccacctactctacatggggacgaacacctgtcatgg
ttctgcagccctgcagcaggctccttcaccaccctggtagcccaagtcca
agtgggcaggccagccccagaccctggaaaaccaggaagggaatccaggc
tcagactgctggggaactattctttgtggttggagggatccaaagaggaa
gatgccgggcggtactggtgcgctgtgctaggtcagcaccacaactacca
gaactggagggtgtacgacgtcttggtgctcaaaggatcccagttatctg
caagggctgcagatggatccccctgcaatgtcctcctgtgctctgtggtc
cccagcagacgcatggactctgtgacctggcaggaagggaagggtcccgt
gaggggccgtgttcagtccttctggggcagtgaggctgccctgctcttgg
tgtgtcctggggaggggctttctgagcccaggagccgaagaccaagaatc
atccgctgcctcatgactcacaacaaaggggtcagctttagcctggcagc
ctccatcgatgcttctcctgccctctgtgccccttccacgggctgggaca
tgccttggattctgatgctgctgctcacaatgggccagggagttgtcatc
ctggccctcagcatcgtgctctggaggcagagggtccgtggggctccagg
cagaggaaaccgaatgcggtgctacaactgtggtggaagccccagcagtt
cttgcaaagaggccgtgaccacctgtggcgagggcagaccccagccaggc
ctggaacagatcaagctacctggaaaccccccagtgaccttgattcacca
acatccagcctgcgtcgcagcccatcattgcaatcaagtggagacagagt
cggtgggagacgtgacttatccagcccacagggactgctacctgggagac
ctgtgcaacagcgccgtggcaagccatgtggcccctgcaggcattttggc
tgcagcagctaccgccctgacctgtctcttgccaggactgtggagcggat
agggggagtaggagtagagaagggaacaagggagcaagggaacaagggac
atctgaacatctaatgtgagaagagaaacatccttctgtgagtcattaaa
atctatgaaccactct

The amino acid sequence for Ly6G6D is set forth below:

Ly6G6D Amino Acid Sequence

(SEQ ID NO:58)

MAVLFLLLFLCGTPQAADNMQAIYVALGEAVELPCPSPPTLHGDEHLSWF
CSPAAGSFTTLVAQVQVGRPAPDPGKPGRESRLRLLGNYSLWLEGSKEED
AGRYWCAVLGQHHNYQNWRVYDVLVLKGSQLSARAADGSPCNVLLCSVVP
SRRNDSVTWQEGKGPVRGRVQSFWGSEAALLLVCPGEGLSEPRSRRPRII
RCLMTHNKGVSFSLAASIDASPALCAPSTGWDMPWILMLLLTMGQGVVIL
ALSIVLWRQRVRGAPGRGNRMRCYNCGGSPSSSCKEAVTTCGEGRPQPGL
EQIKLPGNPPVTLIHQHPACVAAHHCNQVETESVGDVTYPAHRDCYLGDL
CNSAVASHVAPAGILAAAATALTCLLPGLWSG

Analysis of the Ly6G6D protein sequence using the SMART program identified two potential transmembrane domains and an Ig domain, suggesting that this protein is a cell surface protein.

### Example 6

### Identification of Colon-Cancer Associated Gene AI821606

### FLJ32334

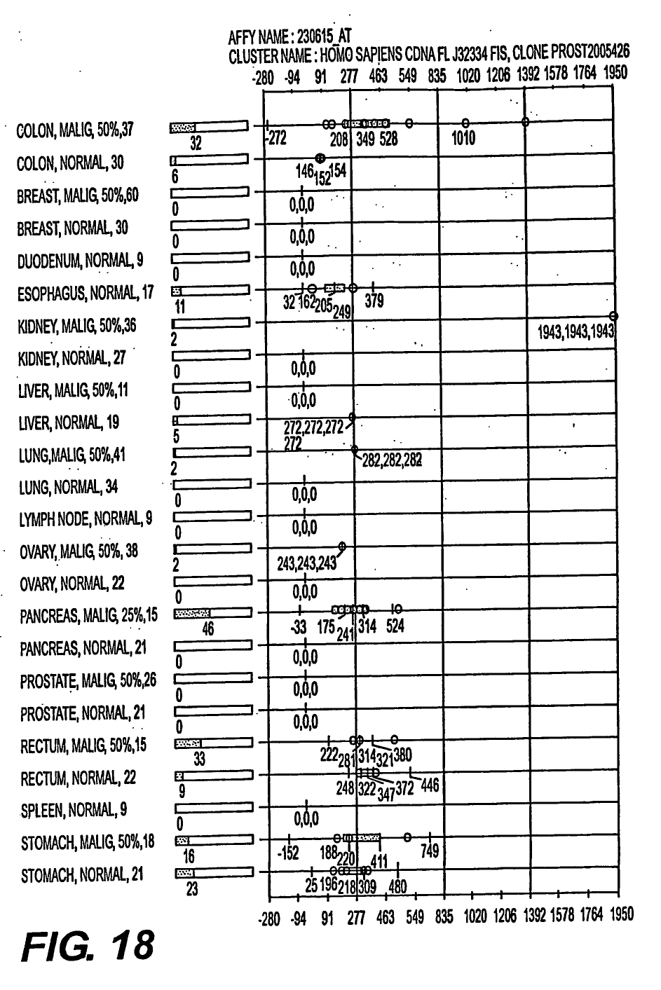

Fragment AI821606 set forth below, was shown to be upregulated in colon, pancreas and rectal malignancies. This is supported by the E-Northern results in FIG. 18.

AI821606 Nucleotide Sequence

(SEQ ID NO:51)

TTCCTCGGAGGGGCCGTGGTGAGTCTCCAGTATGTTCGGCCCAGCGCTCT
TCGCACCCTTCTGGACCAAAGCGCCAAGGACTGCAGCCAGGAGAGAGGGG
GCTCACCTCTTATCCTCGGCGACCCACTGCACAAGCAGGCCGCTCTCCCA
GACTTAAAATGTATCACCACTAACCTGTGAGGGGGACCCAATCTGGACTC
CTTCCCCGCCTTGGGACATCGCAGGCCGGGAAGCAGTGCCCGCCAGGCCT
GGGCCAGGAGAGCTCCAGGAAGGGCACTGAGCGCTGCTGGCGCGAGGCCT
CGGACATCCGCAGGCACCAGGGAAAGTCTCCTGGGGCGATCTGTAAAT

A database search revealed that AI821606 is in the 3′UTR of predicted genes corresponding to both strands of a chromosome. Based thereon, this fragment could be part of the following genes:

ENST00000267803 Nucleotide Sequence

(SEQ ID NO:52)

gcttccagcggacggcagcgcgcgagcattgccccccctgcaccacctca
ccaagATGGCTACTTTGGGACACACATTCCCCTTCTATGCTGGCCCCAAG
CCAACCTTCCCGATGGACACCACTTTGGCCAGCATCATCATGATCTTTCT
GACTGCACTGGCCACGTTCATCGTCATCCTGCCTGGCATTCGGGGAAAGA
CGAGGCTGTTCTGGCTGCTTCGGGTGGTGACCAGCTTATTCATCGGGGCT
GCAATCCTGGGGACCCCCGTGCAGCAGCTGAATGAGACCATCAATTACAA
CGAGGAGTTCACCTGGCGCCTGGGTGAGAACTATGCTGAGGAGTATGCAA
AGGCTCTGGAGAAGGGGCTGCCAGACCCTGTGTTGTACCTAGCTGAGAAG
TTCACTCCAAGAAGCCCATGTGGCCTATACCGCCAGTACCGCCTGGCGGG
ACACTACACCTCAGCCATGCTATGGGTGGCATTCCTCTGCTGGCTGCTGG
CCAATGTGATGCTCTCCATGCCTGTGCTGGTATATGGTGGCTACATGCTA
TTGGCCACGGGCATCTTCCAGCTGTTGGCTCTGCTCTTCTTCTCCATGGC
CACATCACTCACCTCACCCTGTCCCCTGCACCTGGGCGCTTCTGTGCTGC
ATACTCACCATGGGCCTGCCTTCTGGATCACATTGACCACAGGACTGCTG
TGTGTGCTGCTGGGCCTGGCTATGGCGGTGGCCCACAGGATGCAGCCTCA
CAGGCTGAAGGCTTTCTTCAACCAGAGTGTGGATGAAGACCCCATGCTGG
AGTGGAGTCCTGAGGAAGGTGGACTCCTGAGCCCCCGCTACCGGTCCATG
GCTGACAGTCCCAAGTCCCAGGACATTCCCCTGTCAGAGGCTTCCTCCAC
CAAGGCATACTGTAAGGAGGCACACCCCAAAGATCCTGATTGTGCTTTAt
aacattcctccccgtggaggccacctggacttccagtctggctccaaacc
tcattggcgccccataaaaccagcagaactgccctcagggtggctgttac
cagacacccagcaccaatctacagacggagtagaaaaaggaggctctata
tactgatgttaaaaaacaaaacaaaacaaaaagccctaagggactgaaga
gatgctgggcctgtccataaagcctgttgccatgataaggccaagcaggg
gctagcttatctgcacagcaacccagcctttccgtgctgccttgcctctt
caagatgctattcactgaaacctaacttcacccccataacaccagcaggg
tgggggttacatatgattctcctatggtttcctctcatccctcggcacct
cttgttttcctttttcctgggttccttttgttcttcctttacttctccag
cttgtgtggccttttggtacaatgaaagacagcactggaaaggaggggaa
accaaacttctcatcctaggtctaacattaaccaactatgccacattctc
tttgagcttcagttcccaaatttgctacataagattgcaagacttgccaa
gaatcttgggatttatctttctatgccttgctgacacctaccttggccct
caaacaccacctcacaagaagccaggtgggaagttagggaatcaactcca
aaacgctattccttcccaccccactcagctgggctagctgagtggcatcc
aggacgggggagtgggtgacctgcctcatcactgccacctaacgtccccc
tggggtggttcagaaagatgctagctctggtagggtccctccggcctcac
tagagggcgcccctattactctggagtcgacgcagagaatcaggtttcac
agcactgcggagagtgtactaggctgtctccagcccagcgaagctcatga
ggacgtgcgaccccggcgcggagaagccatgaaaattaatgggaaaaaca
gtttttaaaaaacaaaagaaaaaaaggtttatttacagatcgccccagga
gactttccctggtgcctgcggatgtccgaggcctcgcgccagcagcgctc
agtgcccttcctggagctctcctggcccaggcctggcgggcactgcttcc
cggcctgcgatgtcccaaggcggggaaggagtccagattgggtccccctc
acaggttagtggtgatacattttaagtctgggagagcggcctgcttgtgc
agtgggtcgccgaggataagaggtgagccccctctctcctggctgcagtc
cttggcgctttggtccagaagggtgcgaagagcgctgggccgaacatact
ggagactcaccacggcccctccgaggaagaggcacaggacgcctgtggcg
gtggggatcgaaagaaaggagggcatgtggagtcagggctatgttgccca
ggctggtctcgaactctggcctcaaacgaccttcctgcctcgacctccca
aagtgctgggattacaggcgtgatgcccgggccttcttccatcttttgga
gcctaccccttgtgttacctcccgccacacacctctaatctgaattacat
gaaacacggcaagacaccaaacccttctgagccccccacttttcatctgt
aaaatggtcataacagtgcctgtttctgcgaactattgagaggggcaaat
agggtaatagatgtgaattcattctgtaaactgg

The predicted coding sequence for ENST00000267803 is set forth below:

ENST00000267803 Amino Acid Sequence

(SEQ ID NO:53)

MATLGHTFPFYAGPKPTFPMDTTLASIIMIFLTALATFIVILPGIRGKTR
LFWLLRVVTSLFIGAAILGTPVQQLNETINYNEEFTWRLGENYAEEYAKA
LEKGLPDPVLYLAEKFTPRSPCGLYRQYRLAGHYTSAMLWVAFLCWLLAN
VMLSMPVLVYGGYMLLATGIFQLLALLFFSMATSLTSPCPLHLGASVLHT
HHGPAFWITLTTGLLCVLLGLAMAVAHRMQPHRLKAFFNQSVDEDPMLEW
SPEEGGLLSPRYRSMADSPKSQDIPLSEASSTKAYCKEAHPKDPDCAL

SMART analysis predicted that the protein contains several transmembrane domains (rectangles) and a signal sequence, as depicted schematically below:

Based on a sequence contained on the opposite strand of the chromosome, the following gene sequence is predicted:

chr15.41.013.a Nucleotide Sequence

(SEQ ID NO:54)

ATGACCCTGTGGAACGGCGTACTGCCTTTTTACCCCCAGCCCCGGCATGC
CGCAGGCTTCAGCGTTCCACTGCTCATCGTTATTCTAGTGTTTTTGGCTC
TAGCAGCAAGCTTCCTGCTCATCTTGCCGGGGATCCGTGGCCACTCGCGC
TGGTTTTGGTTGGTGAGAGTTCTTCTCAGTCTGTTCATAGGCGCAGAAAT
TGTGGCTGTGCACTTCAGTGCAGAATGGTTCGTGGGTACAGTGAACACCA
ACACATCCTACAAAGCCTTCAGCGCAGCGCGCGTTACAGCCCGTGTCCGT
CTGCTCGTGGGCCTGGAGGGCATTAATATTACACTCACAGGGACCCCAGT
GCATCAGCTGAACGAGACCATTGACTACAACGAGCAGTTCACCTGGCGTC
TGAAAGAGAATTACGCCGCGGAGTACGCGAACGCACTGGAGAAGGGGCTG
CCGGACCCAGTGCTCTACCTGGCGGAGAAGTTCACACCGAGTAGCCCTTG
CGGCCTGTACCACCAGTACCACCTGGCGGGACACTACGCCTCGGCCACGC
TATGGGTGGCGTTCTGCTTCTGGCTCCTCTCCAACGTGCTGCTCTCCACG
CCGGCCCCGCTCTACGGAGGCCTGGCACTGCTGACCACCGGAGCCTTCGC
GCTCTTCGGGGTCTTCGCCTTGGCCTCCATCTCTAGCGTGCCGCTCTGCC
CGCTCCGCCTAGGCTCCTCCGCGCTCACCACTCAGTACGGCGCCGCCTTC
TGGGTCACGCTGGCAACCGGTGAGGACCGAGAGAATGGGCCCCGGGGGCT
AAGGGTGGAGACAGGATTCACACCGGGCGTCCTGTGCCTCTTCCTCGGAG
GGGCCGTGGCCGGGAAGCAGTGCCCGCCAGGCCTGGGCCAGGAGAGCTCC
AGGAAGGGCACTGAGCGCTGCTGGCGCGAGGCCTCGGACATCCGCAGGCA
CCAGGGAAAGTCTCCTGGGGCGATCTGTAAA

This sequence is predicted to encode the following protein:

chr15.41.013.a Amino Acid Sequence

(SEQ ID NO:55)

MTLWNGVLPFYPQPRHAAGFSVPLLIVILVFLALAASFLLILPGIRGHSR
WFWLVRVLLSLFIGAEIVAVHFSAEWFVGTVNTNTSYKAFSAARVTARVR
LLVGLEGINITLTGTPVHQLNETIDYNEQFTWRLKENYAAEYANALEKGL
FDPVLYLAEKFTPSSPCGLYHQYHLAGHYASATLWVAFCFWLLSNVLLST
PAPLYGGLALLTTGAFALFGVFALASISSVPLCPLRLGSSALTTQYGAAF
WVTLATGEDRENGPRGLRVETGFTPGVLCLFLGGAVAGKQCPPGLGQESS
RKGTERCWREASDIRRHQGKSPGAICK

SMART analysis identified three transmembrane domains (rectangles) and a signal sequence. The predicted structure of the protein is depicted schematically below:

### Example 7

### Identification of Cancer Associated Gene CHEM 1

The following DNA sequences were identified as overexpressed in malignant colon tissues as well as other cancers. Expression data was obtained using GENETAG® analysis at Celera/Applied Biosystems as described in Example 1.

The bs243 ms232-222 sequence, set forth below, was initially found to be overexpressed in colon cancer.

bs243ms232-222

(SEQ ID NO:66)

GATCCTGGGACCCCTGGGCCGTGCCTGCCCTCCACCTTGAGTGCCATACT
CCCAACAGCTCCAGGTACCCACCGGGGGATGTGCCTGCTCAGGAAACCTC
TTTGCTCCACACAGCATGGGGCTTCAGCTGCTGGCCCAAGGCCAGGAGCG
CTGGGTTCTGCAGCAGGGCTCAGCCTCAGGGGCGTTA

This sequence corresponds to the 3′UTR of the hypothetical protein Hs16_{—}15516_{—}28_{—}2_{—}1402.a predicted by the Acembly program, C16000171 predicted by the FGENESH program, chr16_{—}148 predicted by the GeneID program and NT_{—}015360.30 predicted by the GeneScan program. The Hs16_{—}15516_{—}28_{—}2_{—}1402a sequence is set forth below, which contains 5′ and 3′ UTRs.

>Hs16_15516_28_2_1402.a

(SEQ ID NO:67)

ccctcccgcgtccggccgcgcccgtcctcctggctgcagagagactaccg
gccaccgccgccgccgccgccgcgagctgtccctgcggcgcgtctgcctt
ggcggagccgaccgcagtgcgctcaggcgtccggtgcgtccccagcctcc
gccccggcgcgggggcgacggactcgcgcgtgcgcagcgccggaggggcg
cgggctgggaccccctagccagcgcgtgcgccgatcgagcgcagggcgat
gggtgggcgccgggcgccgggcgccaggcagtgatgggccttcccgcgct
gcggccccactgaggaggaggctcggggacagcaggagcacgggctgccc
gcgcggtgcggaccATGGCGTTCCTGGCCGGGCCGCGCCTGCTGGACTGG
GCCAGCTCGCCGCCGCACCTGCAGTTCAATAAGTTCGTGCTGACCGGGTA
CCGGCCCGCCAGCAGCGGCTCGGGCTGCCTGCGCAGCCTCTTCTACCTGC
ACAACGAACTGGGCAACATCTACACGCACGGGCTGGCCCTGCTGGGCTTC
CTGGTGCTGGTGCCAATGACCATGCCCTGGGGTCAGCTGGGCAAGGATGG
CTGGCTGGGAGGCACACATTGCGTGGCCTGCCTTGCACCCCCTGCAGGCT
CCGTGCTCTATCACCTCTTTATGTGCCACCAAGGGGGCAGCGCTGTGTAC
GCCCGGCTCCTCGCCCTGGACATGTGTGGGGTCTGCCTTGTCAACACCCT
TGGGGCCCTGCCCATCATCCACTGCACCCTGGCCTGCAGGCCCTGGCTGC
GCCCGGCTGCCCTGGTGGGCTACACTGTGTTGTCGGGTGTGGCCGGCTGG
CGTGCTCTCACCGCCCCCTCCACCAGTGCTCGGCTCCGGGCATTTGGATG
GCAGGCTGCTGCCCGCCTACTGGTATTTGGGGCCCGGGGAGTGGGTCTGG
GTTCAGGGGCTCCAGGCTCCCTGCCCTGCTACCTGCGCATGGACGCACTG
GCGCTGCTTGGGGGACTGGTAAATGTAGCCCGTCTGCCCGAGCGCTGGGG
ACCTGGCCGCTTTGACTACTGGGGCAACTCCCACCAGATCATGCACCTGC
TGAGCGTGGGCTCCATCCTGCAGCTGCACGCCGGCGTCGTGCCCGACCTG
CTCTGGGCTGCCCACCACGCCTGTCCCCGGGACTGAgctgccatgccagc
ctgcccacagcagcctcctagagttagcaacaccaggtgttcctcccaac
tcgtctgcaaggggctggctccttggatgcttccagctcatgagatgtct
cagcaggagccctgttcacccgttcttccctgtggactgacctcttccac
ccacgccgtggcgctccaacttccttccctgccttttccctccaagctcc
tattttactgtgtcagctggaaggaaacctttccctcttgggacctcttt
accctctgtgacctgtggggttagaccagagagggactctggggtcacgt
cttgctctgagagttcaagtcctgccaggccgccagcccagagcctcctc
accctatcctgttcctcccaccaggcctgtggccagtcttcctgatctcc
atctttctgccctgcataccagccctcccagcagccacaagcttgcccgc
cctggctccctctgcccagagactatggagtaaggcattcaggacaaaag
gaccaagggggcgtggacccgtcttgtaccagctggccacaggcacaagg
gctgcagctgcttcttccaggaaactgacacagggagctcagcggcctca
gatcctgggacccctgggccgtgcctgccctccaccttgagtgccatact
cccaacagctccaggtacccaccgggggatgtgcctgctcaggaaacctc
tttgctccacacagcatggggcttcagctgctggcccaaggccaggagcg
ctgggttctgcagcagggctcagcctcaggggcgttaagaccctggatga
catcaataaagggacaggaagggccatgttgccacatgagcaagcttggg
tgctcccaaggttcaaatactttttattagacacggccaggcagagaaga
ccatgggagttcccgaggggccccagctttcaagggcgacgggagagaca
caggataaaaggttaaaagtgcagaggcagagtctggggctcaggttggg
tctagggtgtcctcaaacaggctgaggaggttccgaggctcaaaggaggg
gaaggagccccgaggaggctctgagttgatgtcacttaggtccagggcat
ccctgggaggagagagtagtgacactcaggatccaaaagctagccctgcc
caccccagcccctggacctgcttacctgggtgtgcacctgctccgggggg
tggaggtgctccccacagtccgggccaggacagcctcaggggagagtgaa
ggcctgcaggagggcaggcgagacaaggagggtgtccagggctagggagt
gccggatgaaaccagctctgtccctgtgcaggctccaggctcccgcctga
caaacaggcagggagccacagtcagggacaataaaaacttggtgcactct
gaaagcagcacttggacagccttcaaagtccttccatctggctgcactcc
aaggccccctctgtccttttcagaacacatggacttggaggcagatttga
aataaacttttagtaaatgtaa

HS16_{—}15516_{—}28_{—}2_{—}1402.a encodes the following protein:

>Hs16_15516_28_2_1402.a

(SEQ ID NO:68)

MAFLAGPRLLDWASSPPHLQFNKFVLTGYRPASSGSGCLRSLFYLHNELG
NIYTHGLALLGFLVLVPMTMPWGQLGKDGWLGGTHCVACLAPPAGSVLYH
LFMCHQGGSAVYARLLALDMCGVCLVNTLGALPIIHCTLACRPWLRPAAL
VGYTVLSGVAGWRALTAPSTSARLRAFGWQAAARLLVFGARGVGLGSGAP
GSLPCYLRMDALALLGGLVNVARLPERWGPGRFDYWGNSHQIMHLLSVGS
ILQLHAGVVPDLLWAAHHACPRD

This protein may have between 2 and 6 transmembrane domains, based on sequence analysis using a variety of publicly available transmembrane prediction programs.

Further analysis of the bs243 ms232-222 sequence suggested that there may be an alternatively spliced transcript. This predicted splice variant, UPF0073.5.b is set forth below. UPF0073.5c, d, and e are alternatively spliced transcripts without changes to the coding sequence and are not depicted.

>UPF0073.5.b

(SEQ ID NO:69)

ctggcgtcccctcccgcgtccggccgcgcccgtcctcctggctgcagaga
gactaccggccaccgccgccgccgccgccgcgagctgtccctgcggcgcg
tctgccttggcggagccgaccgcagtgcgctcaggcgtccggtgcgtccc
cagcctccgccccggcgcgggggcgacggactcgcgcgtgcgcagcgccg
gaggggcgcgggctgggaccccctagccagcgcgtgcgccgatcgagcgc
agggcgatgggtgggcgccgggcgccgggcgccaggcagtgatgggcctt
cccgcgctgcggccccactgaggaggaggctcggggacagcaggagcacg
ggctgcccgcgcggtgcggaccATGGCGTTCCTGGCCGGGCCGCGCCTGC
TGGACTGGGCCAGCTCGCCGCCGCACCTGCAGTTCAATAAGTTCGTGCTG
ACCGGGTACCGGCCCGCCAGCAGCGGCTCGGGCTGCCTGCGCAGCCTCTT
CTACCTGCACAACGAACTGGGCAACATCTACACGCACGGCTCCGTGCTCT
ATCACCTCTTTATGTGCCACCAAGGGGGCAGCGCTGTGTACGCCCGGCTC
CTCGCCCTGGACATGTGTGGGGTCTGCCTTGTCAACACCCTTGGGGCCCT
GCCCATCATCCACTGCACCCTGGCCTGCAGGCCCTGGCTGCGCCCGGCTG
CCCTGGTGGGCTACACTGTGTTGTCGGGTGTGGCCGGCTGGCGTGCTCTC
ACCGCCCCCTCCACCAGTGCTCGGCTCCGGGCATTTGGATGGCAGGCTGC
TGCCCGCCTACTGGTATTTGGGGCCCGGGGAGTGGGTCTGGGTTCAGGGG
CTCCAGGCTCCCTGCCCTGCTACCTGCGCATGGACGCACTGGCGCTGCTT
GGGGGACTGGTAAATGTAGCCCGTCTGCCCGAGCGCTGGGGACCTGGCCG
CTTTGACTACTGGGGCAACTCCCACCAGATCATGCACCTGCTGAGCGTGG
GCTCCATCCTGCAGCTGCACGCCGGCGTCGTGCCCGACCTGCTCTGGGCT
GCCCACCACGCCTGTCCCCGGGACTGAgctgccatgccagcctgcccaca
gcagcctcctagagttagcaacaccaggtgttcctcccaactcgtctgca
aggggctggctccttggatgcttccagctcatgagatgtctcagcaggag
ccctgttcacccgttcttccctgtggactgacctcttccacccacgccgt
ggcgctccaacttccttccctgccttttccctccaagctcctattttact
gtgtcagctggaaggaaacctttccctcttgggacctctttaccctctgt
gacctgtggggttagaccagagagggactctggggtcacgtcttgctctg
agagttcaagtcctgccaggccgccagcccagagcctcctcaccctatcc
tgttcctcccaccaggcctgtggccagtcttcctgatctccatctttctg
ccctgcataccagccctcccagcagccacaagcttgcccgccctggctcc
ctctgcccagagactatggagtaaggcattcaggacaaaaggaccaaggg
ggcgtggacccgtcttgtaccagctggccacaggcacaagggctgcagct
gcttcttccaggaaactgacacagggagctcagcggcctcagatcctggg
acccctgggccgtgcctgccctccaccttgagtgccatactcccaacagc
tccaggtacccaccgggggatgtgcctgctcaggaaacctctttgctcca
cacagcatggggcttcagctgctggcccaaggccaggagcgctgggttct
gcagcagggctcagcctcaggggcgttaagaccctggatgacatcaataa
agggacaggaagggccatgttgccacatgagcaagcttgggtgctcccaa
ggttcaaatactttttattagacacggccaggcagagaagaccatgggag
ttcccgaggggccccagctttcaagggcgacgggagagacacaggataaa
aggttaaaagtgcagaggcagagtctggggctcaggttgggtctagggtg
tcctcaaacaggctgaggaggttccgaggctcaaaggaggggaaggagcc
ccgaggaggctctgagttgatgtcacttaggtccagggcatccctgggag
gagagagtagtgacactcaggatccaaaagctagccctgcccaccccagc
ccctggacctgcttacctgggtgtgcacctgctccggggggtggaggtgc
tccccacagtccgggccaggacagcctcaggggagagtgaaggcctgcag
gagggcaggcgagacaaggagggtgtccagggctagggagtgccggatga
aaccagctctgtccctgtgcaggctccaggctcccgcctgacaaacaggc
agggagccacagtcagggacaataaaaacttggtgcactctgaaagcagc
acttggacagccttcaaagtccttccatctggctgcactccaaggccccc
tctgtccttttcagaacacatggacttggaggcagatttgaaataaactt
ttagtaaatgtaagcctt

The amino acid sequence for this splice variant is shown below:

>UPF0073.5.b

(SEQ ID NO:70)

MAFLAGPRLLDWASSPPHLQFNKFVLTGYRPASSGSGCLRSLFYLHNELG
NIYTHGSVLYHLFMCHQGGSAVYARLLALDMCGVCLVNTLGALPIIHCTL
ACRPWLRPAALVGYTVLSGVAGWRALTAPSTSARLRAFGWQAAARLLVFG
ARGVGLGSGAPGSLPCYLRMDALALLGGLVNVARLPERWGPGRFDYWGNS
HQIMHLLSVGSILQLHAGVVPDLLWAAHHACPRD

Analysis of this protein sequence using protein analysis programs suggested that this protein may have one or three transmembrane domains. Although the hemolysin domain in the shorter version was not predicted using SMART, the UPF0073 domain was predicted using Profile with an E value of 4.9e-06.

When the bs243 ms232-222 sequence was searched against the PFAM motif database, (both through the SMART database and the Profile Scan Servers), amino acids 33-259 show homology to UPF0073 (Uncharacterized protein family (Hly-III/UPF0073)) with an E value of 4.8 e-08 (SMART) and 2.8 e-08 (Profile). This novel gene is referred to as “CHEM1” (Colon Hemolysin containing, Expressed in other Malignancies), based on its expression in malignancies other than colon cancer.

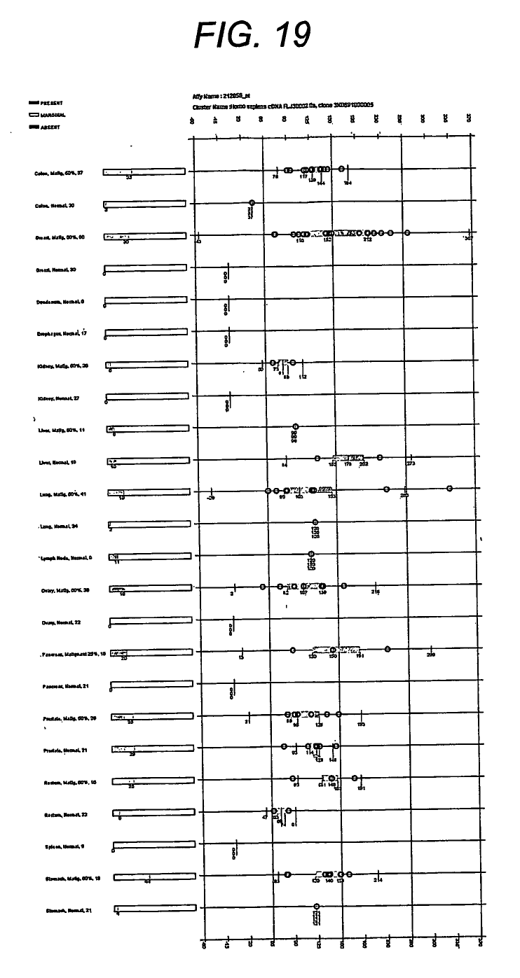

Based on analysis of CHEM1 using the GENE LOGIC® Gene Express datasuite, expression of the CHEM1 gene is upregulated in 30%-45% of breast, colon, prostate, rectum and stomach malignancies. CHEM1 is also detected in 15%-20% of lung, ovary, and pancreatic cancers. Thus, the CHEM1 gene and protein is a useful target for malignancies in a variety of tissues. The electronic northern of the CHEM1 expression obtained using the GENE LOGIC® datasuite is shown in FIG. 19.

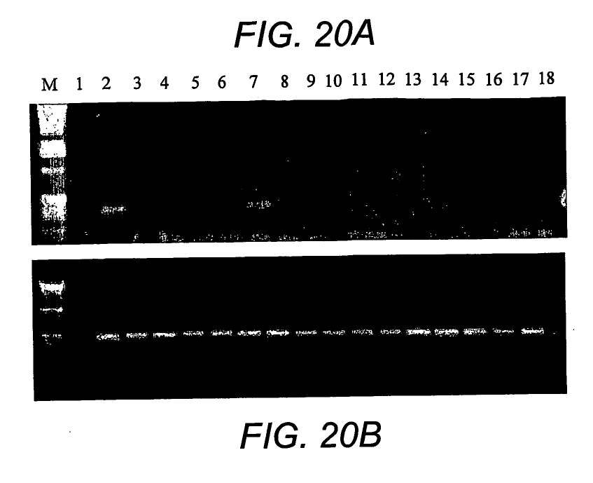

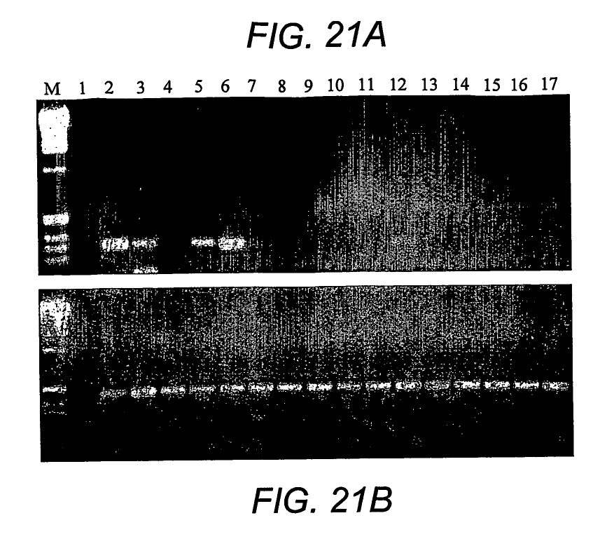

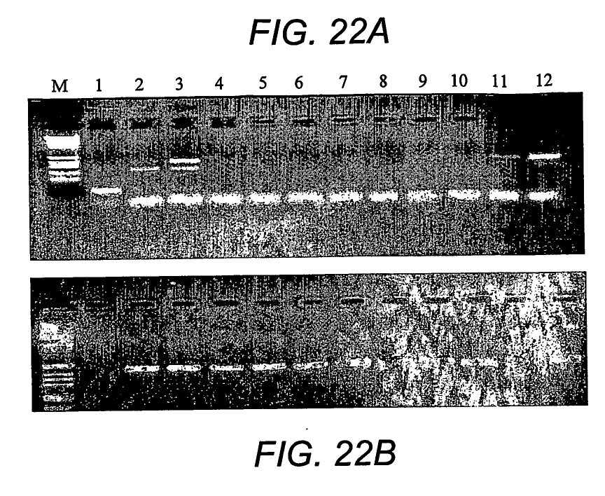

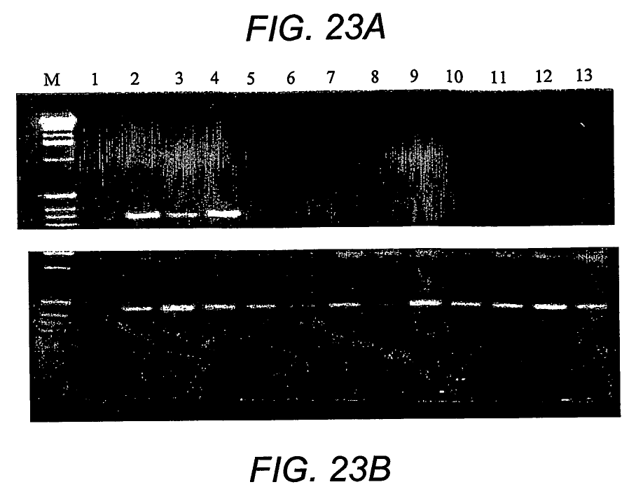

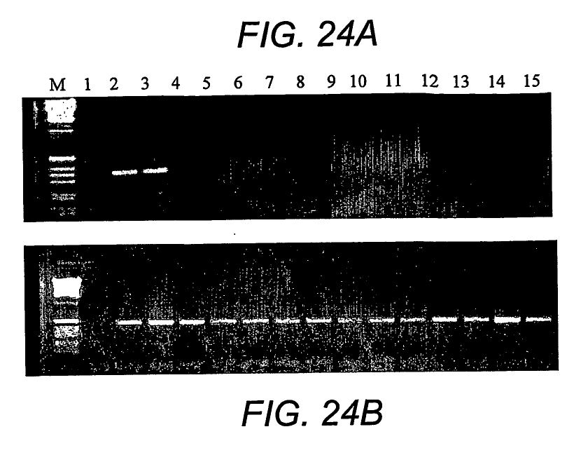

To confirm the data from the GeneExpress program, the expression of CHEM1 in normal and malignant human tissues was determined by PCR experiments using commercially available human cDNA panels (obtained from Clontech and Biochain) and additional cDNA samples prepared from human tissues and cell lines. For preparation of the additional samples, tissue samples were obtained from Grossmont Hospital (LaMesa, Calif.), and cell lines were obtained from ATCC (Manassas, Va.) or the Arizona Cancer Center (Tuscon, Ariz.). RNA from each of the tissues and cell lines was prepared using RNEASY® RNA purification kit (Qiagen). Complementary DNA was synthesized from the RNA templates using SUPERSCRIPT® II cDNA synthesis system (Invitrogen). To amplify CHEM1 products from cDNA samples, short, intron-spanning primers were used to amplify CHEM1 transcripts from multiple tissue panels (Clonetech). Amplification of GAPDH was performed as a control. The CHEM1 message is overexpressed in malignant colon and prostate when compared to normal organs. See FIGS. 20-24.

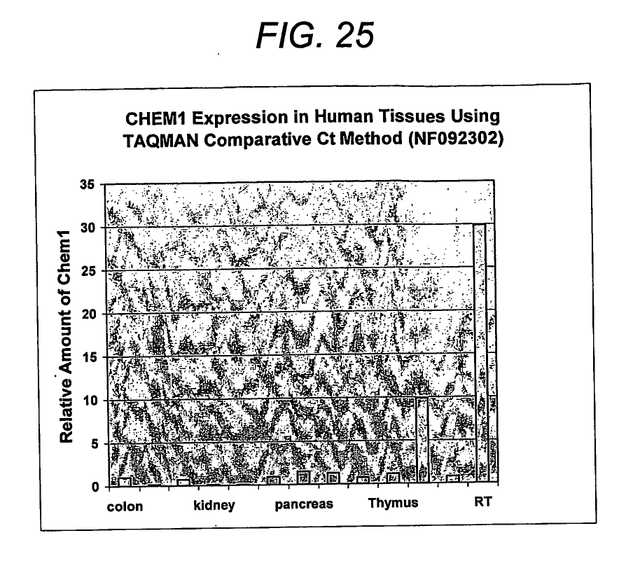

To quantify the levels of CHEM1 transcripts in different tissues, a TAQMAN® assay was performed. Levels of CHEM1 transcripts were compared in prostrate and colon tumor samples from the purchased samples and the prepared samples. As shown in FIG. 25, CHEM1 message is detected at 10-fold higher levels in prostate tumor N and colon tumor R when compared to normal colon.

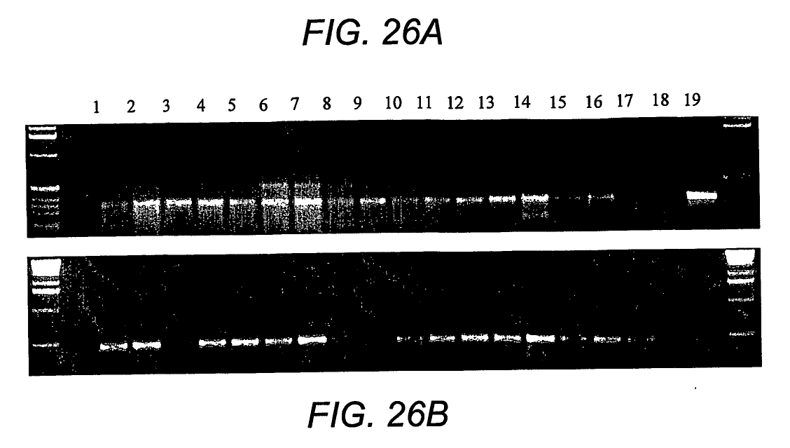

Expression of CHEM1 was also determined in human tumor cell lines using RT-PCR. See FIG. 26. Plasmid DNA from IMAGE clone #4899511 was used as a positive control. Amplification of GAPDH was also performed as a control.

To facilitate development of an animal model for studying CHEM1 function, a murine homolog of human CHEM1 was identified. Animal models are developed using antibodies that target mouse CHEM1, including non-labeled antibodies and antibodies that are conjugated to an effector moiety. For example, an antibody conjugated to a therapeutic radiolabel is used to test the ability of CHEM1 as an appropriate target for cancer therapy, especially for treatment of colon cancer and potentially also breast, rectal, stomach and prostate cancer, given that this protein seems to be overexpressed in these tissues.

The nucleotide sequence of murine CHEM1 is set forth below:

>gi|12963840|ref|NM_023824.1| Mus musculus RIKEN
cDNA 1500004C10 gene (1500004C10Rik), mRNA

(SEQ ID NO:71)

ATGCACTGAGCTCCGACCTGGGGTTGCCAGCTTTCTCTCCCTTGCGGGGG
CGTCGAACTCGCGCGTGCGCAGCGCGTGAGGGAAGGGGGCCGGGACCTCC
TTGCTGACCCGGGCAGGGCCACCGGATAGCCGGAGGTGAATCGGGATGAG
CTTCCCAGCGCTGCAGCTCCACTGAGAAGGAAGCCCAGGCGCAGAGGGTC
GCCGGTCGGCCGCAGTGCGTGAGGCCATGGCATTCCTGACCGGGCCTCGT
CTCCTGGACTGGGCTAGCTCGCCGCCGCACCTGCAGTTCAATAAGTTCGT
ATTAACCGGCTACCGGCCGGCCAGCAGCGGCTCGGGCTGTCTGCGCAGCC
TTTTCTACCTACACAACGAGCTGGGCAACATCTACACACACGGGCTAGCC
CTGCTGGGCTTCCTGGTGTTGGTGCCAATGACCATGCCCTGGAGTCAGCT
GGGCAAGGATGGCTGGCTAGGAGGTACACACTGTGTGGCTTGCCTGGTGC
CCCCTGCAGCCTCTGTGCTGTATCACCTCTTCATGTGCCACCAAGGAGGC
AGTCCTGTGTACACCCGGCTCCTTGCCTTGGATATGTGTGGAGTCTGCCT
TGTCAACACCCTTGGAGCCCTGCCCATCATCCATTGCACTCTGGCCTGCA
GACCGTGGCTTCGCCCAGCTGCCCTGATGGGTTACACTGCACTGTCAGGT
GTAGCCGGCTGGAGAGCTCTCACTGCCCCCTCCACCAGTGCCCGGCTTCG
AGCCTTTGGTTGGCAAGCTGGGGCCCGCCTGCTGGTGTTTGGGGCCCGTG
GAGTGGGGCTGGGCTCAGGGGCTCCAGGCTCTCTGCCCTGCTACCTGCGC
ATGGACGCACTGGCTCTGCTTGGAGGGCTGGTGAATGTGGCACGCCTGCC
AGAGCGGTGGGGGCCTGGTCGCTTCGACTACTGGGGCAACTCCCACCAGA
TCATGCACTTGCTGAGTGTGGGCTCCATCCTCCAGCTCCATGCTGGGGTT
GTGCCTGACCTGCTCTGGGCTGCACACCATGCCTGTCCCCCAGACTGAGC
TGCCTCCTAGCTGCCAAACTGGCTTGCCCACAGCTTCCTGGACAAATTCC
ACCACCTTTCCTCCTACTGGTCTGCAAGGGGCTGGTTCCCTGGAAGAACC
AGCACATGGGACTTCCTAGCTGGGAGACCATTCTTCATTCTTCCCCATGG
ATTCACTTCTTGCATCCAGGCCTTCAAACCCCAGCTTCCACTTTCCTTGC
CATCTTCCCTCCTGGGCATTGTTTTGCTGTCATTAGAAGGAAACCATTTT
TTTTTTTCCCAATTTACCCTGTTTAACCTGTGAGAGTCTCTGACAGTTGA
GTCCTGCCAACTTACCAAGCCTCCAGCCCAGAACCACTACCCCTATGTTG
CTGCTCCCATACATAACTACACCTCCTGCTCCTGGATTCTTGAGCTAGCC
ACTCTGACCCTGCTTCCTGACCTCCATCTCCCTGCTCTGCATGTCAAACC
TCTCAGCAGCCAGAATTTTGCTGTTCCTGTCATTCCTGCAGTGAGGATGC
AGAGGAGTGGGACCAGGCTTCTCTCAGAGCCAAGTGGACATTGGTCCTGC
TTGTATCATCTGGCCAGGAGACAGGAGGGGAACTGCTGCTTTTCCTAGGC
AACAGGCACAGCTGTGGAATGGAGGTGTTGGATTCGGGCTTCACTGGACC
AAGGACTCAGCTCTTCAGTGCCATGGTCTGACTGACCTGCCTACCAGAGA
CTTGTCTGCTCAGGAAATCTCTATACAGTGGGTGGCTCCAGCCTGCTGGC
CCAAGGGTACTGACTCGCAGCCAGATCATCCCAAAGGCCCAAGACCCTAG
GCAACATCAATAAAGGGACAAGAAGAGCTATGCTGCCACATGAGCAACCT
TGGGTGTTCCCAAGACGCATTACTTTTTATTAGACACGGAAGTTTCAGGG
GAGAGGTGGGCAAGACGGTCAGAGGTTTAAAAGCACCAAGGCTGGCTGGG
CCTGTGCTCAGGCTGGGTCTAGGGAGTCCTCAAACAGGCTGAGGAGGTTC
CTTGGCTCAAAGGTGGGGCAGGGACCTCTTGGAGGCTCTGAGTCCACATC
AGTTAGGTCCAGGGCATCCCTTGGGGGAGGAAGAAGAAGAAAAAAAAAAA
AAAAAAAAAGGCCACA

The murine CHEM1 protein is set forth below:

>gi|12963841|ref|NP_076313.1| RIKEN cDNA
1500004C10 [Mus musculus]

(SEQ ID NO:72)

MAFLTGPRLLDWASSPPHLQFNKFVLTGYRPASSGSGCLRSLFYLHNELG
NIYTHGLALLGFLVLVPMTMPWSQLGKDGWLGGTHCVACLVPPAASVLYH
LFMCHQGGSPVYTRLLALDMCGVCLVNTLGALPIIHCTLACRPWLRPAAL
MGYTALSGVAGWRALTAPSTSARLRAFGWQAGARLLVFGARGVGLGSGAP
GSLPCYLRMDALALLGGLVNVARLPERWGPGRFDYWGNSHQIMHLLSVGS
ILQLHAGVVPDLLWAAHHACPPD

Monoclonal antibodies to CHEM1 were generated by immunizing female Balb/c mice with a 16-amino acid peptide corresponding to the C-terminal sequence of CHEM1, coupled to BSA.

Sera titers were measured by ELISA on microtiter plates coated with CHEM1/ovalbumin. Spleens were removed from mice showing the highest titers and fused to mouse myeloma Sp2/0 cells, essentially as described by Kohler & Milstein (1975) Nature 256:495. The resulting hybridomas were initially screened for binding to CHEM1/ovalbumin. Positively reacting sera were subsequently tested on ovalbumin alone and ovalbumin coupled to irrelevant peptides. Selected clones were subcloned by limiting dilution and then allowed to expand in ISPRO media (Irvine Scientific) supplemented with 5% low IgG FBS (Hyclone), HT, and 1% cloning factor. Antibodies were purified from culture supernatants by protein-A affinity chromatography.

CHEM1 expression was detected in a variety of human cell lines by Western blotting using antibodies prepared as described above. See FIG. 26. Whole cell lysates were prepared from the following tumor cell lines: NCI-H69 (small cell lung cancer), ZR-75-1 (breast cancer), MDA-MB-468 (breast cancer, adenocarcinoma), AsPC-1, HT-29 (colon cancer, colorectal adenocarcinoma), LS 174T and HCT116. Protein concentration of the lysates were determined using the DC Protein Assay kit (BioRad) according to the manufacturer's instructions. The cell lysates (50 μg) were resolved by SDS-PAGE and subjected to immunoblotting using purified anti-CHEM1 monoclonal antibodies (10 μg/ml). The bound anti-CHEM1 antibody was detected using HRP-conjugated anti-mouse IgG secondary antibody (BioRad; 1:1,000) and ECL reagent (Amersham Pharmacia Biotech).

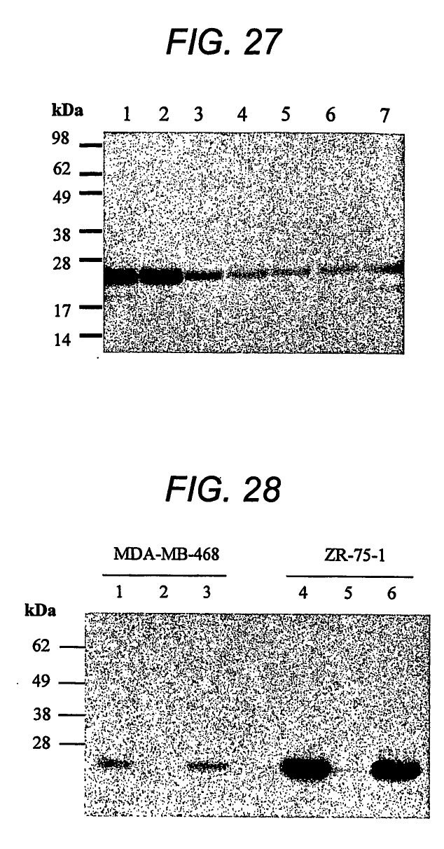

To demonstrate that CHEM1 is a membrane protein, anti-CHEM1 antibodies were used to detect CHEM1 protein in cellular fractions, including post-nuclear supernatant (PNS), cytosol, and membrane fractions from cultured MDA-MB-468 or ZR-75-1 human tumor cell lines. See FIG. 27. One confluent 15-cm culture plate of MDA-MB-468 or ZR-75-1 breast cancer cell lines was washed once with ice-cold PBS followed by two washes with 15 ml of HEES buffer (0.255 M sucrose, 1 mM EDTA, 2 mM EGTA, 10 mM HEPES, pH 7.4). The cells were scraped from the dishes in 1 ml HEES buffer supplemented with a protease inhibitor cocktail (0.1 mg/ml AEBSF, 2 μg/ml aprotinin, 40 μg/ml bestatin, 10 μg/ml chymostatin, 10 μg/ml E-64, 2 μg/ml leupeptin, 2 μg/ml Pepstatin A) using a rubber policeman. The cells were passed five times through a 1-ml ball homogenizer, and centrifuged at 1,000×g for 10 minutes to obtain a post-nuclear supernatant (PNS). The PNS (500 μl) was centrifuged at 100,000×g for 30 minutes to yield membrane (pellet) and cytosol (supernatant) fractions. The membrane fraction was resuspended in 500 μl of HEES buffer supplemented with the protease inhibitor cocktail. The cell fractions (40 μl) were resolved by SDS-PAGE and analyzed by immunoblotting using anti-CHEM1 monoclonal antibody as described above.

1. An isolated nucleic acid expressed by human cancer cells comprising: (i) the nucleotide sequence of SEQ ID NO: 67 or 69; (ii) a nucleotide sequence that is at least 90% identical to SEQ ID NO: 67 or 69; (iii) a nucleotide sequence that is complementary to (i) or (ii); or (iv) a fragment of (i), (ii), or (iii) having a size of at least 20 nucleotides in length.

2. The isolated nucleic acid of claim 1 comprising the nucleotide sequence of SEQ ID NO: 67 or 69, or a fragment thereof.

3. A primer mixture comprising primers that result in the specific amplification of any one of the nucleic acids of claim 1.

4. An antigen expressed by human cancer cells comprising: (i) an antigen encoded by the nucleic acid of SEQ ID NO: 67 or 69; (ii) an antigen having the amino acid sequence of SEQ ID NO: 68 or 70; or (iii) a fragment or variant of (i) or (ii).

5. A human cancer antigen of claim 4 encoded by the nucleic acid of SEQ ID NO: 67 or 69.

6. A human cancer antigen of claim 4 comprising the amino acid sequence of SEQ ID NO: 68 or 70.

7. monoclonal antibody or antigen-binding fragment thereof that specifically binds to a cancer antigen of claim 4.

8. The monoclonal antibody of claim 7, wherein the antibody is a domain deleted antibody.

9. The domain deleted antibody of claim 8, wherein the antibody lacks a C_{H}2 domain.

10. The monoclonal antibody of claim 7, further comprising a detectable label, wherein the detectable label is attached directly or indirectly to the antibody.

11. The monoclonal antibody of claim 7, further comprising a therapeutic agent, wherein the therapeutic agent is attached directly or indirectly to the antibody.

12. The monoclonal antibody of claim 11, wherein the therapeutic agent is a cytotoxin, a growth factor, or a drug.

13. The monoclonal antibody of claim 12, wherein the cytotoxin is a therapeutic radiolabel.

14. The monoclonal antibody of claim 13 wherein the therapeutic radiolabel is ⁹⁰yttrium.

15. The monoclonal antibody of claim 13 wherein the therapeutic radiolabel is ¹¹¹indium.

16. A diagnostic kit for detecting cancer comprising an isolated nucleic acid according to claim 1 and a detectable label.

17. A diagnostic kit for detecting cancer comprising primers according to claim 3 and a diagnostically acceptable carrier.

18. A diagnostic kit for detecting cancer comprising a monoclonal antibody according to claim 10.

19. A method of detecting cancer comprising (i) obtaining a human cell sample; and (ii) determining whether such cell sample expresses a cancer gene having a nucleotide sequence of SEQ ID NO: 67 or 69.

20. The method of claim 19, wherein said method comprises detecting the expression of the cancer gene using a nucleic acid that specifically hybridizes thereto.

21. The method of claim 19, wherein said method comprises detecting the expression of the cancer gene using primers that result in the amplification thereof.

22. The method of claim 19, wherein the expression of said cancer gene is detected by performing an assay to detect the presence or level of the antigen encoded by said gene.

23. The method of claim 22, wherein the assay involves use of a monoclonal antibody, or antigen-binding fragment thereof.

24. The method of claim 11, wherein the assay comprises an ELISA or competitive binding assay.

25-27. (canceled)

28. A method for treating cancer in a subject comprising administering to the subject a therapeutically effective amount of a therapeutic agent selected from the group consisting of: (a) a cancer antigen encoded by the nucleic acid of SEQ ID NO: 67 or 69 or fragment or variant thereof; (b) a ribozyme or antisense oligonucleotide that inhibits the expression of the gene having the nucleotide sequence of SEQ ID NO: 67 or 69 or fragment or variant thereof; (c) a cancer antigen comprising the amino acid sequence of SEQ ID NO: 68 or 70 or fragment or variant thereof; and (d) a ligand which specifically binds to any one of (a) or (c) and an adjuvant.

29-38. (canceled)


SEQUENCE LISTING (73 sequences)

SEQ ID NO: 1 - DNA, 149 bp, Homo sapiens
gatccaggagaggaaggagtttcagaaggcaggagctggtcctctatgtcatgaaatgtagagggtgaggccaaggaggacctgagagaaggtaattagatttggtgtttacaggctggtccctgtggccagccaccccacccacttta

SEQ ID NO: 2 - DNA, 679 bp, Homo sapiens
tgaggaaactgtggcttagaggaaaaggtcattagttcattttgggatttgttgattttcagatgtttgagatgttgaggatggattgtccagcaggctattaagatgtggtgaaggctagaaatgttgatttaggaggtattgccttcgagaagataaaggaggagaagaggagagcatcatgcaagctagagaagagaaagaagaaaagtattctggggaatgtctcctttgggagcagaaagaagactctgacggagcagccatccaggaagtggaatgagatccaggagaggaaggagtttcagaaggcaggagctggtcctctatgtcatgaaatgtagagggtgaggccaaggaggacctgagagaaggtaattagatttggtgtttacaggctggtccctgtggccagccaccccacccactttaaaatatttactctacaaatgttaatgtgtgaagagttgcatgccagaatatttatggcatcagtgttggtggatacagaacattgggaaacaacccattaatagcagaatggtaaatctggccagtgaatagtatagctttttaaaaggaggctgatgtctgaattcactttcaaagttgttcacaatgtattgctaaaatacaaaaatgttgcagaaccatatgtatgagagaaacccctttttct

SEQ ID NO: 3 - DNA, 155 bp, Homo sapiens
gatccccatggtatgcttgaatctgctccctgaacttcctgccagtgcctccccgtaccccaaaacaatgtcaccatggttaccacctacccagaagactgttccctcctcccaagacccttgtctgcagtggtgctcctgcaggctgcccgtta

SEQ ID NO: 4 - DNA, 1795 bp, Homo sapiens
agtgtggtgatggttgtcttcgacaatgagaaggtcccagtagagcagctgcgcttctggaagcactggcattcccggcaacccactgccaagcagcgggtcattgacgtggctgactgcaaagaaaacttcaacactgtggagcacattgaggaggtggcctataatgcactgtcctttgtgtggaacgtgaatgaagaggccaaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcggtctggaggggcagccccctcggcaggacccagcagctccaacaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctgtaaggcctctcgagcatccaaaccctcacgacctgcaaggggccagcagggacgtggccccacgccacacacaacctctccacatgcctcagcgctgttacttgaatgccttccctgagggaagaggcccttgagtcacagacccacagacgtcagggccagggagagacctagggggtcccctggcctggatccccatggtatgcttgaatctgctccctgaacttcctgccagtgcctccccgtaccccaaaacaatgtcaccatggttaccacctacccagaagactgttccctcctcccaagacccttgtctgcagtggtgctcctgcaggctgcccgttaagatggtggcggcacacgctccctcccgcagcaccacgccagctggtgcggcccccactctctgtcttccttcaacttcagacaaaggatttctcaacctttggtcagttaacttgaaaactcttgattttcagtgcaaatgacttttaaaagacactatattggagtctctttctcagacttcctcagcgcaggatgtaaatagcactaacgatcgactggaacaaagtgaccgctgtgtaaaactactgccttgccactcactgttgtatacatttcttatttacgattttcatttgttatatatatatataaatatactgtatatatatgcaacattttatatttttcatggatatgtttttatcatttcaaaaaatgtgtatttcacatttcttggactttttttagctgttattcagtgatgcattttgtatactcacgtggtatttagtaataaaaatctatctatgtattacgtcac

SEQ ID NO: 5 - protein, 322 aa, Homo sapiens
SVVMVVFDNEKVPVEQLRFWKHWHSRQPTAKQRVIDVADCKENFNTVEHIEEVAYNALSFVWNVNEEAKVFIGVNCLSTDFSSQKGVKGVPLNLQIDTYDCGLGTERLVHRAVCQIKIFCDKGAERKMRDDERKQFRRKVKCPDSSNSGVKGCLLSGFRGNETTYLRPETDLETPPVLFIPNVHFSSLQRSGGAAPSAGPSSSNRLPLKRTCSPFTEEFEPLPSKQAKEGDLQRVLLYVRRETEEVFDALMLKTPDLKGLRNAISEKYGFPEENIYKVYKKCKRGILVNMDNNIIQHYSNHVAFLLDMGELDGKIQIILKEL

SEQ ID NO: 6 - DNA, 1782 bp, Homo sapiens
aagttgccccacctctctgagcattggcttccccatctgtgaaagaggagtgctgatgtttgccttctaggggcctagtgaggcttaagggtgagcagcaggcacacagaaagctagaaatacaggatcactgtgggacggtggggctggccacctgggcaggccacttacccagcggccccctctgtctccaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcggtctggaggggcagccccctcggcaggacccagcagctccaacaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctgtaaggcctctcgagcatccaaaccctcacgacctgcaaggggccagcagggacgtggccccacgccacacacaacctctccacatgcctcagcgctgttacttgaatgccttccctgagggaagaggcccttgagtcacagacccacagacgtcagggccagggagagacctagggggtcccctggcctggatccccatggtatgcttgaatctgctccctgaacttcctgccagtgcctccccgtaccccaaaacaatgtcaccatggttaccacctacccagaagactgttccctcctcccaagacccttgtctgcagtggtgctcctgcaggctgcccgttaagatggtggcggcacacgctccctcccgcagcaccacgccagctggtgcggcccccactctctgtcttccttcaacttcagacaaaggatttctcaacctttggtcagttaacttgaaaactcttgattttcagtgcaaatgacttttaaaagacactatattggagtctctttctcagacttcctcagcgcaggatgtaaatagcactaacgatcgactggaacaaagtgaccgctgtgtaaaactactgccttgccactcactgttgtatacatttcttatttacgattttcatttgttatatatatatataaatatactgtatatatatgcaacattttatatttttcatggatatgtttttatcatttcaaaaaatgtgtatttcacatttcttggactttttttagctgttattcagtgatgcattttgtatactcacgtggtatttagtaataaaaatctatctatgtattacgtcac

SEQ ID NO: 7 - protein, 195 aa, Homo sapiens
MRDDERKQFRRKVKCPDSSNSGVKGCLLSGFRGNETTYLRPETDLETPPVLFIPNVHFSSLQRSGGAAPSAGPSSSNRLPLKRTCSPFTEEFEPLPSKQAKEGDLQRVLLYVRRETEEVFDALMLKTPDLKGLRNAISEKYGFPEENIYKVYKKCKRGILVNMDNNIIQHYSNHVAFLLDMGELDGKIQIILKEL

SEQ ID NO: 8 - DNA, 1458 bp, Homo sapiens
atgaaaaggtctgtgcggctgctaaagaacgacccagtcaacttgcagaaattctcttacactagtgaggatgaggcctggaagacgtacctagaaaacccgttgacagctgccacaaaggccatgatgagagtcaatggagatgatgagagtgttgcggccttgagcttcctctatgattactacatgtcgatgctcttcccagatatcctgaaaacctccccggaacccccatgtccagaggactaccccagcctcaaaagtgactttgaatacaccctgggctcccccaaagccatccacatcaagtcaggcgagtcacccatggcctacctcaacaaaggccagttctaccccgtcaccctgcggaccccagcaggtggcaaaggccttgccttgtcctccaacaaagtcaagagtgtggtgatggttgtcttcgacaatgagaaggtcccagtagagcagctgcgcttctggaagcactggcattcccggcaacccactgccaagcagcgggtcattgacgtggctgactgcaaagaaaacttcaacactgtggagcacattgaggaggtggcctataatgcactgtcctttgtgtggaacgtgaatgaagaggccaaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcggtctggagggagcctccagcagccaggggctcctctcattttcctgcgtgtgatggaaaatgtctttttcacttcattgcaggcagccccctcggcaggacccagcagctccaacaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctgtaa

SEQ ID NO: 9 - protein, 485 aa, Homo sapiens
MKRSVRLLKNDPVNLQKFSYTSEDEAWKTYLENPLTAATKAMMRVNGDDESVAALSFLYDYYMSMLFPDILKTSPEPPCPEDYPSLKSDFEYTLGSPKAIHIKSGESPMAYLNKGQFYPVTLRTPAGGKGLALSSNKVKSVVMVVFDNEKVPVEQLRFWKHWHSRQPTAKQRVIDVADCKENFNTVEHIEEVAYNALSFVWNVNEEAKVFIGVNCLSTDFSSQKGVKGVPLNLQIDTYDCGLGTERLVHRAVCQIKIFCDKGAERKMRDDERKQFRRKVKCPDSSNSGVKGCLLSGFRGNETTYLRPETDLETPPVLFIPNVHFSSLQRSGGSLQQPGAPLIFLRVMENVFFTSLQAAPSAGPSSSNRLPLKRTCSPFTEEFEPLPSKQAKEGDLQRVLLYVRRETEEVFDALMLKTPDLKGLRNAISEKYGFPEENIYKVYKKCKRGILVNMDNNIIQHYSNHVAFLLDMGELDGKIQIILKEL

SEQ ID NO: 10 - DNA, 2100 bp, Homo sapiens
atggaggcaggggagaaaagcgctctgggtgcctggagcccgcagccctgggcagccccgggctaccgcagggcgcaagggatcctgggctgcggccgagggcgccggaagtcgccgccgaccgcctgggtctcgcaggaaaacagccggcgcccgcgagctgcccagcgtcgggttttcctgaagagcccagctcctcacaccttggggcctggtgggatgggagacactgtcctggatgaagccgctgggagagctgccgcctcctgtatgctgaggtctgtgcggctgctaaagaacgacccagtcaacttgcagaaattctcttacactagtgaggatgaggcctggaagacgtacctagaaaacccgttgacagctgccacaaaggccatgatgagagtcaatggagatgatgagagtgttgcggccttgagcttcctctatgattactacatgggtcccaaggagaagcggatattgtcctccagcactgggggcaggaatgaccaaggaaagaggtactaccatggcatggaatatgagacggacctcactccccttgaaagccccacacacctcatgaaattcctgacagagaacgtgtctggaaccccagagtacccagatttgctcaagaagaataacctgatgagcttggagggggccttgcccacccctggcaaggcagctcccctccctgcaggccccagcaagctggaggccggctctgtggacagctacctgttacccaccactgatatgtatgataatggctccctcaactccttgtttgagagcattcatggggtgccgcccacacagcgctggcagccagacagcaccttcaaagatgacccacaggagtcgatgctcttcccagatatcctgaaaacctccccggaacccccatgtccagaggactaccccagcctcaaaagtgactttgaatacaccctgggctcccccaaagccatccacatcaagtcaggcgagtcacccatggcctacctcaacaaaggccagttctaccccgtcaccctgcggaccccagcaggtggcaaaggccttgccttgtcctccaacaaagtcaagagtgtggtgatggttgtcttcgacaatgagaaggtcccagtagagcagctgcgcttctggaagcactggcattcccggcaacccactgccaagcagcgggtcattgacgtggctgactgcaaagaaaacttcaacactgtggagcacattgaggaggtggcctataatgcactgtcctttgtgtggaacgtgaatgaagaggccaaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcggtctggagggctccaactgcctagttaccggccgcaggaccatctgcaattcccagcccttctgggcatgctggggcccaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctgtaa

SEQ ID NO: 11 - protein, 650 aa, Homo sapiens
MEAGEKSALGAWSPQPWAAPGYRRAQGILGCGRGRRKSPPTAWVSQENSRRPRAAQRRVFLKSPAPHTLGPGGMGDTVLDEAAGRAAASCMLRSVRLLKNDPVNLQKFSYTSEDEAWKTYLENPLTAATKAMMRVNGDDESVAALSFLYDYYMGPKEKRILSSSTGGRNDQGKRYYHGMEYETDLTPLESPTHLMKFLTENVSGTPEYPDLLKKNNLMSLEGALPTPGKAAPLPAGPSKLEAGSVDSYLLPTTDMYDNGSLNSLFESIHGVPPTQRWQPDSTFKDDPQESMLFPDILKTSPEPPCPEDYPSLKSDFEYTLGSPKAIHIKSGESPMAYLNKGQFYPVTLRTPAGGKGLALSSNKVKSVVMVVFDNEKVPVEQLRFWKHWHSRQPTAKQRVIDVADCKENFNTVEHIEEVAYNALSFVWNVNEEAKVFIGVNCLSTDFSSQKGVKGVPLNLQIDTYDCGLGTERLVHRAVCQIKIFCDKGAERKMRDDERKQFRRKVKCPDSSNSGVKGCLLSGFRGNETTYLRPETDLETPPVLFIPNVHFSSLQRSGGLQLPSYRPQDHLQFPALLGMLGPRLPLKRTCSPFTEEFEPLPSKQAKEGDLQRVLLYVRRETEEVFDALMLKTPDLKGLRNAISEKYGFPEE

SEQ ID NO: 12 - DNA, 186 bp, Homo sapiens
gatctgcaatcagaactattgaacttctccattcagaccgccactcacacctatgggaaaagggtaatgtatcatcggcttagcaacagggaatactattcgtatgatggaaaatggggacaaaaggctttggtacataaaacattattccttccttggcctaaaaactcatcgccacctacatta

SEQ ID NO: 13 - DNA, 2333 bp, Homo sapiens
tctggagcagctgaaaaacaaggaagtgaaacagccaattcctgccttaactaattaacccaccttacgacattccaccattatgacgtgttcctgccctgccccaactgatcaatcgaccctgtgacattcttctggacaatgagtcccatcatctctccaccatgcaccttgtgactccctcctctgctgacaacagataaccacctttaactgtaactttccacagcctaccccagccctataaagctgcccctctcctatctcccttcgctgactctcttttcagactcagcccacttgcacccaagtgaattaacagccttgttgctcacacaaagcctgtttaggtggtcttctatacggacatgcttgacacttggtgccaaaatctgggccagggggactccttcgtgagaccggccccctgtcctggccctcattccgtgaagagatccacctgcgacctcgggtcctcagaccagcccaaggaacatctcaccaatttcaaatcggatctcctcggcttagtggctgaagactgatgctgcccgatcgcctcagaagccccttggaccatcacagatgccgagcttcgggtaactcttacggtggaggattcccagccatatgaagacaccctagctggacgatcagtccttgtcaaaagtctgacccctcaaactctacagcctcaatggaccagaccctacccggtcatttatagcacaccaactgccgtccatctgcaggaccctctccattgggttcaccattccagaataaagccatgcccatcagacagccagcttgatctctcctcttcctcctggaagccacaagattaggccgagagccgatcagacaaacaacctacaacccttaagctcctggcagcgcccagccaaggccatgcttccttgcaacactccttccaaatggccatcccagcatgcttccaagcaggcttcatccgttcctctggaccctcatctcttaagacctgccgcctataaaaaggattatatcttgagaccctatcctctaaaattttttccacacccaaaacaaaaaatctctgggtcaaaagtctaaaacgcttaggctggcaaccatcagatccttgcccatggtgtcctcaagcctactctcatgaaatggacaacagtacacgcatatggggccagttccacatatttggcaaccagaccagcatccaggacaacacaaagatctgcaatcagaactattgaacttctccattcagaccgccactcacacctatgggaaaagggtaatgtatcatcggcttagcaacagggaatactattcgtatgatggaaaatggggacaaaaggctttggtacataaaacattattccttccttggcctaaaaactcatcgccacctacattaaagctaatatgcctgattactgtttttagagaacttattttattagggcagttccaagctcaaaaatacgctaactggcaccttgttagctacataaaaatgcaccctagacccgaaacttactagactcattataaaattttctttaaggtgtccacgcagtccctggtcacacttgaagcagtccggagaaatatcagccctaccccagtaatccccagaaggaacttacacttttttttaatcttttcctacaacttcatattttataaataaaaagacaaaaatgtcaggcctgtgagctgaagcttagccattgtaacccctgtgacctgcacatatccgtccaggtggcctgcaggagccaagaagtctggagcagccgaaaaaccacaaagaagtgaaacagccagttcctgccttaactaattaacccaccttacgacattccaccattatgacttgtccaccattatgacttgttcctgccctgccccaactgatcaatcaaccctgtgacattcttctcctggacaatgagtcccatcatctctccaccatgcaccttgtgaccccctcctctgctgaggataaccacctttaactgtaactttccacgcctacccaagccctataaagctgcccctctcctatctcccttcactgactctcttttcggactcagcccacttgcacccaagtgaattaacagccttgttgctcacacaaagcctgattgggtgtcttctatacggacacgcgtgacaggaacctcaacccaaaggcagtctgatgaggtgtctaagataaaagtagcggcacaaaggcttttgtaaacagaggcgtttcatgtggttttcctttcctttccttatatgtgaaaaggtgacagaaaagaaatcttcctaaaagagtc

SEQ ID NO: 14 - protein, 121 aa, Homo sapiens
MGPVPHIWQPDQHPGQHKDLQSELLNFSIQTATHTYGKRVMYHRLSNREYYSYDGKWGQKALVHKTLFLPWPKNSSPPTLKLICLITVFRELILLGQFQAQKYANWHLVSYIKMHPRPETY

SEQ ID NO: 15 - DNA, 2436 bp, Homo sapiens
tctggagcagctgaaaaacaaggaagtgaaacagccaattcctgccttaactaattaacccaccttacgacattccaccattatgacgtgttcctgccctgccccaactgatcaatcgaccctgtgacattcttctggacaatgagtcccatcatctctccaccatgcaccttgtgactccctcctctgctgacaacagataaccacctttaactgtaactttccacagcctaccccagccctataaagctgcccctctcctatctcccttcgctgactctcttttcagactcagcccacttgcacccaagtgaattaacagccttgttgctcacacaaagcctgtttaggtggtcttctatacggacatgcttgacacttggtgccaaaatctgggccagggggactccttcgtgagaccggccccctgtcctggccctcattccgtgaagagatccacctgcgacctcgggtcctcagaccagcccaaggaacatctcaccaatttcaaatcggatctcctcggcttagtggctgaagactgatgctgcccgatcgcctcagaagccccttggaccatcacagatgccgagcttcgggtaactcttacggtggaggattcccagccatatgaagacaccctagctggacgatcagtccttgtcaaaagtctgacccctcaaactctacagcctcaatggaccagaccctacccggtcatttatagcacaccaactgccgtccatctgcaggaccctctccattgggttcaccattccagaataaagccatgcccatcagacagccagcttgatctctcctcttcctcctggaagccacaagattaggccgagagccgatcagacaaacaacctacaacccttaagctcctggcagcgcccagccaaggccatgcttccttgcaacactccttccaaatggccatcccagcatgcttccaagcaggcttcatccgttcctctggaccctcatctcttaagacctgccgcctataaaaaggattatatcttgagaccctatcctctaaaattttttccacacccaaaacaaaaaatctctgggtcaaaagtctaaaacgcttaggctggcaaccatcagatccttgcccatggtgtcctcaagcctactctcatgaaatggacaacagtacacgcatatggggccagttccacatatttggcaaccagaccagcatccaggacaacacaaagtatgttgtttgttgttagagggcttgggacatttcactctttgccagcctcagcttaatccaggagacaaagattattttccttattatctcttctgcataggatctgcaatcagaactattgaacttctccattcagaccgccactcacacctatgggaaaagggtaatgtatcatcggcttagcaacagggaatactattcgtatgatggaaaatggggacaaaaggctttggtacataaaacattattccttccttggcctaaaaactcatcgccacctacattaaagctaatatgcctgattactgtttttagagaacttattttattagggcagttccaagctcaaaaatacgctaactggcaccttgttagctacataaaaatgcaccctagacccgaaacttactagactcattataaaattttctttaaggtgtccacgcagtccctggtcacacttgaagcagtccggagaaatatcagccctaccccagtaatccccagaaggaacttacacttttttttaatcttttcctacaacttcatattttataaataaaaagacaaaaatgtcaggcctgtgagctgaagcttagccattgtaacccctgtgacctgcacatatccgtccaggtggcctgcaggagccaagaagtctggagcagccgaaaaaccacaaagaagtgaaacagccagttcctgccttaactaattaacccaccttacgacattccaccattatgacttgtccaccattatgacttgttcctgccctgccccaactgatcaatcaaccctgtgacattcttctcctggacaatgagtcccatcatctctccaccatgcaccttgtgaccccctcctctgctgaggataaccacctttaactgtaactttccacgcctacccaagccctataaagctgcccctctcctatctcccttcactgactctcttttcggactcagcccacttgcacccaagtgaattaacagccttgttgctcacacaaagcctgattgggtgtcttctatacggacacgcgtgacaggaacctcaacccaaaggcagtctgatgaggtgtctaagataaaagtagcggcacaaaggcttttgtaaacagaggcgtttcatgtggttttcctttcctttccttatatgtgaaaaggtgacagaaaagaaatcttcctaaaagagtc

SEQ ID NO: 16 - protein, 100 aa, Homo sapiens
CCPIASEAPWTITDAELRVTLTVEDSQPYEDTLAGRSVLVKSLTPQTLQPQWTRPYPVIYSTPTAVHLQDPLHWVHHSRIKPCPSDSQLDLSSSSWKPQD

SEQ ID NO: 17 - DNA, 517 bp, Homo sapiens
  misc_feature (1)..(517): n is a, c, g, or t
ggcttctaaggtacattatgttttactttaataaataaaaattaacttgaagaaaaatgcagngccctatttaattgctctgcatgaaatgtacagaaacggcaacctctgcgattctaagcactgtgaacgccccagccacaccgtgtcaacaaaccgtgtggcacttgggagaaggcaggggtgatttacgantagtcatgtttcgcctccacccgagtcactgccaaggagtggacagtgacactgaataagcatncggngcacctccttcgggaagggacttggctgacatggtaggccttcccactggagcctgtactttgtcttgctgggcagcactccantcatgggaaggaacaatgancaaggcgtggtggtgggggtgngtaggcctgagcgccgttttccatggtgaccttcactgagcaggcagcaggcactgatgggcagttgagnctggnaggagtcaggtcctggtcntgcctctggtgtaacgcagcangccatcaaaggt

SEQ ID NO: 18 - DNA, 766 bp, Homo sapiens
  misc_feature (1)..(766): n is a, c, g, or t
agaattcggcacgagnttttttttctcttagatctccaggttcccttccttaccccgggaagcctttcttcatcccaccgtcctggggcgttncacagtgcttagaatcgcagaggttgccgtttctgtacatttcatgcagagcaattaaatagggcactgcatttttcttcaagttaatttttatttattaaagtaaaacataatgtaccttagaagccagacagtcctacaagcttattatgttgtacagcggcgttccgtccccctccccagccctctctttctagaggcagccaatttcagctgtctctctctgcttacctacatatttccatgtttcttggttcatcacctggtggcaccttcagtctggaaacacctgcccttcactttaggggaattgggcccctgttcgtttgataagttttcctaccattttctgatttgttttttctttctggaaaatgtattagtcagatgtaggcttttctggattaatccttcaactttcctttctttctttcccttcctgcctgtctccctgttctttcttacactttctcagggagattcttgactgtattttccaactttgtatcgaccattttacttttcctgccatattttcaatgtttactgatgtttctctgccctttcagtgcatcctggttttatttcatgttagactgaatccatgtgaaattgataacaggttttcagcccacacacacacacacaaaaaaaaaaaaaaaaaaaaaaaaaa

SEQ ID NO: 19 - DNA, 455 bp, Homo sapiens
ttttgttggctgaggcggtattttccttttattgctgttatgagattcaacattttttccagaaataacttctgaaaagtgtgcctagattttgaacacttgtgatcctaacatgtggtgagaaaggcttttcaaaacacacacgtgtggacagaggtccacacacggatacgtgtgcacacacgggtgccttgggcgtgcgtcttccaaaaggggcgagtacagctatcaacttgtgacttccaggaggcctgggtttgcctacgaaggggccgtgttcccagttggcgttcacacgtggtgtacacacacaggcacaggcaccgtgtcccaaggccatctcccaagggcacccgcagacactgggcagccttctccgaagctgtcagtgtccttcctcgtgagaggatgatgaagaggatgtggtttccgccgcctcatccacaggccggctg

SEQ ID NO: 20 - DNA, 1225 bp, Homo sapiens
  misc_feature (1)..(1225): n is a, c, g, or t
naaaanggcgccngncccanntaaaatnnacccncctaaaggggaaaaactnnggcggccgccttcgtttttttttttttttttttgtggtggctgaggcggtattttccttttattgctgttaagagattcaacattttttccagaaataacttctgaaaagggggcctnagattttgaacacttgggatcctaacagggggtgagaaaggcttttcaaaacacacnacgggtggacagaggtccacacacggnatacgggggcacacacgggtgccttgggcgtgcgtcttccaaaaggggcgagntacagctatcaacttgtgacttccaggaggcctgggtttgcctacgaaggggccgntgttcccagttggcgttcacacgtggtgtacacacacaggcacaggcaccngtgtcccaanggccatctncccaagggcacccgcagacactgggcagccttctccgaagctgtcagtgtccttcctcgtgagaggatgatgaagaggatgtggtttccgccgcctcatccacaggccggctgcccacggagccttagacatcgaggccagagcgacagaagcctgtgtgctgaccggcctggtctcctttgacgtctcgagcagcttggcagggtgggaaaagtagcctgagagtgatccccgggcagtgtccgaggctctgccgtccccacccccacaggcatccaggggagagaaacaacctgcgcctgcgaggccgtgcggaccccgctccactcaccccgcctggggggccagaaccacctcccaggggcttccgccagtgccgcagttgctgaccccaggcaaacctcgccgcctcctgccccggcgggcctgggatttgcgaatgtgtgaaggcattagctgccagttgtaactggaacccagcctagaggcctcactcctccagcaggaagccttgtaatgcagcgaatctgaacccggcccagcgtccagagacaggaagcattaataggagcgaatgtgaacactgttcgcgccctggctgcgatttattgccgattgtggggaaaacatcagttggttgcagagtttcattcatctttagggacaggaccggtgtgtctgggtggcagtttagagagctgggacagtcggcatcactctgggtggctcctctcaanccctggtgcctcgtgccgaattctggcctcgaggcattctnaggggctntatnc

SEQ ID NO: 21 - DNA, 308 bp, Homo sapiens
ttttttttttttgtggataaatatattagcaaatgaatatatttcttaacatagtgcctgattcaagcgtctgtctggttcaaatataaatacccatgtgggtacctaggtgctagtctccccactaactgagggaaaaaggttcccaggtggggtcctctgcccactttgccaccacattcacattccaaatgggataatgcctgaggggccatgagtggtcaggctgccctggggtgaatgtcaccctgatgaggcccatcagctcttgtccactcagtgaggccagacttgtgctctaatccact

SEQ ID NO: 22 - DNA, 1212 bp, Homo sapiens
  misc_feature (1)..(1212): n is a, c, g, or t
ctntgtanaaagctgggtacgcgtaagcttgggcccctcgagggatactctagagcggccgccctttttttttttttttgtggataaatatattagcaaataaatatatttcttaacatagtgcctgattcaagcgtctgtctggttcagatataaatacccatgtgggtacctaggtgctagtctccccactaactgagggaaaaaggttcccaggtggggtcctctgcccactttgccaccacattcacattccaaatgggataatgcctgaggggccaagagtggtcaggctgccctggggtgaatgtcaccctgatgaggcccatcagctcttgtccactcagtgaggccagacttgtgctctaatccactctcctgtgggtccctggcctgtatggcttatactggggagctgggcctctgggctgtccaaacccaagggtcacactttgcttttcctttgttgtccccattttccatccttgctctaagacaaaacttttcccagagaagaactctttgttgtccccgctcagctgtaattctgccttttctaccttcattccatccttcctctgcccagataaagtccagcagaaattcctcctttctacctctctgggactctgagacaggaaatcttcaaggaggagtttttccctccccactattcttattctcaacccccagaagaaccaanggctgctgtacccccctcagggacagaactccacactatangggggaaagnttcangggaccccttccttttantgctcanggctccacctatgctactggntccttttggcaaaaaaggnaaatganagagccaggggttgccccntgatgtaacanccnttactggggangggnccaangnnggtgntcaaagnnccccnaggagggaggnganaaggggtcatgngttctgctnaanccnctggttggtataaanttgangnttggggtganggaaaccaaaaanggntggaaaaagnaaaacacctttnnaaaccctgggtaccnnanataagnttttggcccnaaaaantcngccnncaagggatccgccccncccccccagggaaaaanttggttcctngggngaaaaggantttnccccccncaaattttnnccnaaaagntttggaanttgnaaaanaaaagganccttcccccccccnccacaaaaaaaaaaaaaaaaaa

SEQ ID NO: 23 - DNA, 1229 bp, Homo sapiens
  misc_feature (1)..(1229): n is a, c, g, or t
cnnttncaaaaagcaggctggtaccggtccggaattcccgggatatcgtcgacccacgccgtccggtttgctggtgttgctgaaataactccagcagaaggaaaattaatgcagtcccacccgctgtacctgtgcaatgccagtgatgacgacaatctggagcctggattcatcagcatcgtcaagctggagagtcctcgacgggccccccgcccctgcctgtcactggctagcaaggctcggatggcgggtgagcgaggagccagtgctgtcctctttgacatcactgaggatcgagctgctgctgagcagctgcagcagccgctggggctgacctggccagtggtgttgatctggggtaatgacgctgagaagctgatggagtttgtgtacaagaaccaaaaggcccatgtgaggattgagctgaaggagcccccggcctggccagattatgatgtgtggatcctaatgacagtggtgggcaccatctttgtgatcatcctggcttcggtgctgcgcatccggtgccgcccccgccacagcaggccggatccgcttcagcagagaacagcctgggccatcagccagctggccaccaggaggtaccaggccagctgcaggcaggcccggggtgagtggccagactcagggagcagctgcagctcagcccctgtgtgtgccatctgtctggagggagttctctgagggggcaggagctacgggtcatttccctgcctccatgagttccatcgtaactgtgtggacccctggntacatcagcatccggacttgccccctcttgcatggttcaacatcacanaggggagatccnttttcccngtccctgggaacctctncnatcttaccaagaaccagggtcggaagactcccccctcatttcnccagcatccccggcatgncccactacaccntccctggtngcctacctgttngggcccttccccggaatgcaggggntngggcccccncnaactgggtcctttcctgccntccaggnagccaggcatgggccccccgaatcaccccttccccnaanatggannatcccccgggttccaggaaaacaaacaaccnctggaaggaanccnnnaccccntnncccnaaggctggggaangnaacncccccnattccccntnnanganccctnngtttncncnaggcccctnacccgggccnngcccccnaaacaaaggganttganaaant

SEQ ID NO: 24 - DNA, 3780 bp, Homo sapiens
aaaaaaaaaaaactttagagaaaggaagggccaaaactacgacttggctttctgaaacggaagcataaatgttcttttcctccatttgtctggatctgagaacctgcatttggtattagctagtggaagcagtatgtatggttgaagtgcattgctgcagctggtagcatgagtggtggccaccagctgcagctggctgccctctggccctggctgctgatggctaccctgcaggcaggctttggacgcacaggactggtactggcagcagcggtggagtctgaaagatcagcagaacagaaagctgttatcagagtgatccccttgaaaatggaccccacaggaaaactgaatctcactttggaaggtgtgtttgctggtgttgctgaaataactccagcagaaggaaaattaatgcagtcccacccactgtacctgtgcaatgccagtgatgacgacaatctggagcctggattcatcagcatcgtcaagctggagagtcctcgacgggccccccgcccctgcctgtcactggctagcaaggctcggatggcgggtgagcgaggagccagtgctgtcctctttgacatcactgaggatcgagctgctgctgagcagctgcagcagccgctggggctgacctggccagtggtgttgatctggggtaatgacgctgagaagctgatggagtttgtgtacaagaaccaaaaggcccatgtgaggattgagctgaaggagcccccggcctggccagattatgatgtgtggatcctaatgacagtggtgggcaccatctttgtgatcatcctggcttcggtgctgcgcatccggtgccgcccccgccacagcaggccggatccgcttcagcagagaacagcctgggccatcagccagctggccaccaggaggtaccaggccagctgcaggcaggcccggggtgagtggccagactcagggagcagctgcagctcagcccctgtgtgtgccatctgtctggaggagttctctgaggggcaggagctacgggtcatttcctgcctccatgagttccatcgtaactgtgtggacccctggttacatcagcatcggacttgccccctctgcgtgttcaacatcacagagggagattcattttcccagtccctgggaccctctcgatcttaccaagaaccaggtcgaagactccacctcattcgccagcatcccggccatgcccactaccacctccctgctgcctacctgttgggcccttcccggagtgcagtggctcggcccccacgacctggtcccttcctgccatcccaggagccaggcatgggccctcggcatcaccgcttccccagagctgcacatccccgggctccaggagagcagcagcgcctggcaggagcccagcacccctatgcacaaggctggggaatgagccacctccaatccacctcacagcaccctgctgcttgcccagtgcccctacgccgggccaggccccctgacagcagtggatctggagaaagctattgcacagaacgcagtgggtacctggcagatgggccagccagtgactccagctcagggccctgtcatggctcttccagtgactctgtggtcaactgcacggacatcagcctacagggggtccatggcagcagttctactttctgcagctccctaagcagtgactttgaccccctagtgtactgcagccctaaaggggatccccagcgagtggacatgcagcctagtgtgacctctcggcctcgttccttggactcggtggtgcccacaggggaaacccaggtttccagccatgtccactaccaccgccaccggcaccaccactacaaaaagcggttccagtggcatggcaggaagcctggcccagaaaccggagtcccccagtccaggcctcctattcctcggacacagccccagccagagccaccttctcctgatcagcaagtcaccggatccaactcagcagccccttcggggcggctctctaacccacagtgccccagggccctccctgagccagcccctggcccagttgacgcctccagcatctgccccagtaccagcagtctgttcaacttgcaaaaatccagcctctctgcccgacacccacagaggaaaaggcgggggggtccctccgagcccacccctggctctcggccccaggatgcaactgtgcacccagcttgccagatttttccccattacacccccagtgtggcatatccttggtccccagaggcacaccccttgatctgtggacctccaggcctggacaagaggctgctaccagaaaccccaggcccctgttactcaaattcacagccagtgtggttgtgcctgactcctcgccagcccctggaaccacatccacctggggaggggccttctgaatggagttctgacaccgcagagggcaggccatgcccttatccgcactgccaggtgctgtcggcccagcctggctcagaggaggaactcgaggagctgtgtgaacaggctgtgtgagatgttcaggcctagctccaaccaagagtgtgctccagatgtgtttgggccctacctggcacagagtcctgctcctgggaaaggaaaggaccacagcaaacaccattctttttgccgtacttcctagaagcactggaagaggactggtgatggtggagggtgagagggtgccgtttcctgctccagctccagaccttgtctgcagaaaacatctgcagtgcagcaaatccatgtccagccaggcaaccagctgctgcctgtggcgtgtgtgggctggatcccttgaaggctgagtttttgagggcagaaagctagctatgggtagccaggtgttacaaaggtgctgctccttctccaacccctacttggtttccctcaccccaagcctcatgttcataccagccagtgggttcagcagaacgcatgacaccttatcacctccctccttgggtgagctctgaacaccagctttggcccctccacagtaaggctgctacatcaggggcaaccctggctctatcattttccttttttgccaaaaggaccagtagcataggtgagccctgagcactaaaaggaggggtccctgaagctttcccactatagtgtggagttctgtccctgaggtgggtacagcagccttggttcctctgggggttgagaataagaatagtggggagggaaaaactcctccttgaagatttcctgtctcagagtcccagagaggtagaaaggaggaatttctgctggactttatctgggcagaggaaggatggaatgaaggtagaaaaggcagaattacagctgagcggggacaacaaagagttcttctctgggaaaagttttgtcttagagcaaggatggaaaatggggacaacaaaggaaaagcaaagtgtgacccttgggtttggacagcccagaggcccagctccccagtataagccatacaggccagggacccacaggagagtggattagagcacaagtctggcctcactgagtggacaagagctgatgggcctcatcagggtgacattcaccccagggcagcctgaccactcttggcccctcaggcattatcccatttggaatgtgaatgtggtggcaaagtgggcagaggaccccacctgggaacctttttccctcagttagtggggagactagcacctaggtacccacatgggtatttatatctgaaccagacagacgcttgaatcaggcactatgttaagaaatatatttatttgctaatatatttat

SEQ ID NO: 25 - protein, 783 aa, Homo sapiens
MSGGHQLQLAALWPWLLMATLQAGFGRTGLVLAAAVESERSAEQKAVIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCVFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGMSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTGSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 26 - DNA, 422 bp, Homo sapiens
tttttttttaaacattaagattttattacaaaccaggcattatatatttctttacacttaaggaatagatatgaaacaatcttggagtaaaaattagaaggcaacttgcttcaagtttgtaccaagtcaatcaagcagaaacctgaagaaccttgttttaagatgagagtcatttatacttggcaggcattttcttccaatgaaaaaataaagtcaatgtgccattatcttgacacttataaaaatgtttataaaaagcatttaggccattgattctcacagttggctgaatattggaatcacctagattaaaaaaaatactaatccctatacaacatccccaaaattcagatttaattagtgtaagttaggccctgggcatataggctgttttaaaattcctcgggtgagtctaatgtgta

SEQ ID NO: 27 - DNA, 1219 bp, Homo sapiens
  misc_feature (1)..(1219): n is a, c, g, or t
cccnncnnccnnnnnngnnnnncttanctcgcagncanaattcggccacgcagggtcgccttcgccgccatggnacgccaccgggcgctgacagacctatggagagtcagggtgtgcctcccgggccttatcgggccaccaagctgtggaatgaagttaccacatcttttcgagcaggaatgcctctaagaaaacacagacaacactttaaaaaatatggcaattgtttcacagcaggagaagcagtggattggctttatgacctattaagaaataatagcaattttggtcctgaagttacaaggcaacagactatccaactgttgaggaaatttcttaagaatcatgtaattgaagatatcaaagggaggtggggatcagaaaatgttgatgataacaaccagctcttcagatttcctgcaacttcgccacttaaaactctaccacgaaggtatccagaattgagaaaaaacaacatagagaacttttccaaagataaagatagcatttttaaattacgaaacttatctcgtagaactcctaaaaggcatggattacatttatctcaggaaaatggcgagaaaataaagcatgaaataatcaatgaaagatcaagaaaatgcaattgataatagagaactaagccaggaagatgttgaaagaagnttgggagatatgttattctgatcctacctgcaaaccattttaaggtgtgcccatcccctagaagnaagttcttaaatcccaaaccaggtaattcccccaantanttaatgnacaaacatggnccaatacaagttaanccngggagtagttnttactacaaaaccaattcngatgaccttcccccacnggntntttnnctngccatggaaangnccctaccaaantggcccaanaanncantgatttggaataatccnncctttggttgggattnnancaaattgantccnaannatccccaaatantttncnaaannctccctgancccnacctanctttggaanttncccaattntttggcaaacnttttgggganggaaagaattctccggatttnagcccttntggcaaaggntncacctnnnttnaatttnaagannnacaccctnggnaaatntaanggggcccccnnattntttnaaatncgcggaanaagntcccaggntcccntntttccccccaaaatnnnattgggattcctnacccccccan

SEQ ID NO: 28 - DNA, 1226 bp, Homo sapiens
  misc_feature (1)..(1226): n is a, c, g, or t
cnnnnnantgcggccgctcattttttttttttttttttctctatgnaagcagactgnagnaagaaggcactcagnttgatttgaaggaattcaaattgtttaagtgaaggaattttgaagactgtggatcatcttgaattttatgtatcccactggatctatctgaaactgtgatgtagccacaaacaactaccaggaaatgaaacaaaaattaagatgcaactgtatgacagtggacaaaaataaaacaaaaacaatagtaaagttaaaaaataaagcattactatagtatatattgttagtatagtatacacagtagttgcttaattcagaagccacttaaataggacacatgcaacattcggttacaaacgtgcaagacagatgagtggttttcccatttgtaatataactttaaaaaattatttcaacagcctaattaaatggattgagccagaatacatttaaaaaatctgttctcagtctgcaagtactagaaacctcataaatataagataattgtggtataataaaatacatatatttgatctttgtccttggtacctggtatggagctcctaaaatccttgaaatttcctgaatgatagaagtctttagttactcataacaagcctatttcagcgntatcctgagtttcatgcctaanggtaactganggccnggccatgggtttgaattttcatccaccaactacaacccttgtggggaggagaaagggnctagaaattnaagttcnnttggnccaccagtgacccaatgaattgggtccngtcatgccttggntanttaaaccttccaattaaaacncntaaaacatgcnaggctgangggagttttntagggtnnnggaanccttgnatggggctgggnatccccggattgacccagaaanggtaaaaaaaacncttnggccccccccccccccctnacccggggncttgggaaacccctccctttggccntttnctggaggncnacccttttnaaataaactaaaagccatagntaaaggggcnttttnctnnttnctgggaancttgnanggaatttttngacccnggnaaggggntttgagggaaancccaantnggtaattggcngggcgggaatttnnatacccccngaacccnattncncggaattaaaaaaatttnggnncggncccctttntntnnnccaggggtnaaanttctcnaaannanaaa

SEQ ID NO: 29 - DNA, 1119 bp, Homo sapiens
  misc_feature (1)..(1119): n is a, c, g, or t
agctcgnagccagattcggcacgagggagattatatgttttatttatcattgtctctgcatatctggaacaacgaaaggcacatagcagttgctaaataaatatcttttgaatgaatatatgattgccttatacttcttttatatccccatcttctaatagattatgaaaactagaattcaaaatatatatactgaacaaatgaatgactgaagcaattggggataatatttaaggcaaaaccaaatctgataaaatatacacatattttaaaaacacatacatatatataaatagatcaaaagtggaaaaagaatatataaaagagtgcaacatttggcagctgagaattatttcattgagttttcaaatattcttcacattcttatacttagaaacaaagaagtaaccccaaacaactaattcattagctaatatctcagaacttgcacatttgcagataaattttcttttaagaacagaattatagtttaatccctaacacagctcagttttcaaaattcaagtaaataaaattttagcacacatcatgatagccttactggnatagctgtgttaaaaacaaaaagtatttggtatcatctattgttatgtgctctcaattgagatctagttagtttcctaagagtctcacattgatanctattttgggcacttccttacataatgngnttatttagaaataccttattaatgacagacttccttttgagtagctacattctcagatatggctncatttatcaaagttccccnaggattacctaattttaattccagttagntatctaaactacggaactttnggntttccttaaantcaacattggttgccttgattggaaggnttggcncccaaaaanggcggncntcccncncccgggggtggnaantcttttcntgaanntnccaaggnnaattccctccngaaancnggntttaanttttttnccntttcccccttnaangggaaacccccgggttttnaaaaaaatttttcccaaaanattcnnccnatgggcccctttggaaaggnaaaaanttttttgtcccttaaaaanccctggnaaccnaatttggttnancaaatanaggaagg

SEQ ID NO: 30 - DNA, 1058 bp, Homo sapiens
  misc_feature (1)..(1058): n is a, c, g, or t
gcggccgctgggcctgngtgtcgccttcgccgccatggncgccaccgggcgctgacagacctatggagagtcagggtgtgcctcccgggccttatcgggccaccaagctgtggaatgaagttaccacatcttttcgagcaggaatgcctctaagaaaacacagacaacactttaaaaaatatggcaattgtttcacagcaggagaagcagtggattggctttatgacctattaagaaataatagcaattttggtcctgaagttacaaggcaacagactatccaactgttgaggaaatttcttaagaatcatgtaattgaagatatcaaagggaggtggggatcagaaaatgttgatgataacaaccagctcttcagatttcctgcaacttcgccacttaaaactctaccacgaaggtatccagaattgagaaaaaacaacatagagaacttttccaaagataaagatagcatttttaaattacgaaacttatctcgtagaactcctaaaaggcatggattacatttatctcaggaaaatggcgagaaaataaagcatgaaataatcaatgaagatcaagaaaatgcaattgataatagagaactaagccaggaagatgttgaagaagtttgggagatatgttattctgatctacctgcaaaccattttaggtgtgccatccctagaagaagtcataaatcccaaacaagtaattccccaatatataatgtacnacatggccaatacangtaacgtgggagtagttatactacaaacaaatcagatgacctccctcactgggtattatctgccatgaagngcctagcaaatnggccagaagcatgatatgnaataatccacctttgnnggatttgaccganatgtnttngaacatcccgattatttctaaacccctgaccnctnntactttgaaatnanaattattgnaanctttgggntgctncnccctttaaaggggtgccnccaagcctnngttngtgntgttactncccccaancgaaaagnncnctttatgggtgntncccaagaacaatntnn

SEQ ID NO: 31 - DNA, 2396 bp, Homo sapiens
gtgccgagactcaccactgccgcggccgctgggcctgagtgtcgccttcgccgccatggacgccaccgggcgctgacagacctatggagagtcagggtgtgcctcccgggccttatcgggccaccaagctgtggaatgaagttaccacatcttttcgagcaggaatgcctctaagaaaacacagacaacactttaaaaaatatggcaattgtttcacagcaggagaagcagtggattggctttatgacctattaagaaataatagcaattttggtcctgaagttacaaggcaacagactatccaactgttgaggaaatttcttaagaatcatgtaattgaagatatcaaagggaggtggggatcagaaaatgttgatgataacaaccagctcttcagatttcctgcaacttcgccacttaaaactctaccacgaaggtatccagaattgagaaaaaacaacatagagaacttttccaaagataaagatagcatttttaaattacgaaacttatctcgtagaactcctaaaaggcatggattacatttatctcaggaaaatggcgagaaaataaagcatgaaataatcaatgaagatcaagaaaatgcaattgataatagagaactaagccaggaagatgttgaagaagtttggagatatgttattctgatctacctgcaaaccattttaggtgtgccatccctagaagaagtcataaatccaaaacaagtaattccccaatatataatgtacaacatggccaatacaagtaaacgtggagtagttatactacaaaacaaatcagatgacctccctcactgggtattatctgccatgaagtgcctagcaaattggccaagaagcaatgatatgaatgatccaacttatgttggatttgaacgagatgtattcagaacaatcgcagattattttctagatctccctgaacctctacttacttttgaatattacgaattatttgtaaacattttggttgtttgtggctacatcacagtttcagatagatccagtgggatacataaaattcaagatgatccacagtcttcaaaattccttcacttaaacaatttgaattccttcaaatcaactgagtgccttcttctcagtctgcttcatagagaaaaaaacaaagaagaatcagattctactgagagactacagataagcaatccaggatttcaagaaagatgtgctaagaaaatgcagctagttaatttaagaaacagaagagtgagtgctaatgacataatgggaggaagttgtcataatttaatagggttaagtaatatgcatgatctatcctctaacagcaaaccaaggtgctgttctttggaaggaattgtagatgtgccagggaattcaagtaaagaggcatccagtgtctttcatcaatcttttccgaacatagaaggacaaaataataaactgtttttagagtctaagcccaaacaggaattcctgttgaatcttcattcagaggaaaatattcaaaagccattcagtgctggttttaagagaacctctactttgactgttcaagaccaagaggagttgtgtaatgggaaatgcaagtcaaaacagctttgtaggtctcagagtttgcttttaagaagtagtacaagaaggaatagttatatcaatacaccagtggctgaaattatcatgaaaccaaatgttggacaaggcagcacaagtgtgcaaacagctatggaaagtgaactcggagagtctagtgccacaatcaataaaagactctgcaaaagtacaatagaactttcagaaaattctttacttccagcttcttctatgttgactggcacacaaagcttgctgcaacctcatttagagagggttgccatcgatgctctacagttatgttgtttgttacttcccccaccaaatcgtagaaagcttcaacttttaatgcgtatgatttcccgaatgagtcaaaatgttgatatgcccaaacttcatgatgcaatgggtacgaggtcactgatgatacataccttttctcgatgtgtgttatgctgtgctgaagaagtggatcttgatgagcttcttgctggaagattagtttctttcttaatggatcatcatcaggaaattcttcaagtaccctcttacttactagactgctagtggataataacatcttgactacttaaaaaagggacatattgaaaatcctggagatggactatttgctcctttgcctaacttactcatactgtaagcagattagtgctcaggagtttgatgagcaaaaagtttctacctctcaagctgcaattgctagaactctttagaaaatattattaaaatacaggagtttaccttaaaggaaaaaaaaaaaacaaaaaaaaaaaaaaaaaa

SEQ ID NO: 32 - protein, 692 aa, Homo sapiens
MESQGVPPGPYRATKLWNEVTTSFRAGMPLRKHRQHFKKYGNCFTAGEAVDWLYDLLRNNSNFGPEVTRQQTIQLLRKFLKNHVIEDIKGRWGSENVDDNNQLFRFPATSPLKTLPRRYPELRKNNIENFSKDKDSIFKLRNLSRRTPKRHGLHLSQENGEKIKHEIINEDQENAIDNRELSQEDVEEVWRYVILIYLQTILGVPSLEEVINPKQVIPQYIMYNMANTSKRGVVILQNKSDDLPHWVLSAMKCLANWPRSNDMNDPTYVGFERDVFRTIADYFLDLPEPLLTFEYYELFVNILVVCGYITVSDRSSGIHKIQDDPQSSKFLHLNNLNSFKSTECLLLSLLHREKNKEESDSTERLQISNPGFQERCAKKMQLVNLRNRRVSANDIMGGSCHNLIGLSNMHDLSSNSKPRCCSLEGIVDVPGNSSKEASSVFHQSFPNIEGQNNKLFLESKPKQEFLLNLHSEENIQKPFSAGFKRTSTLTVQDQEELCNGKCKSKQLCRSQSLLLRSSTRRNSYINTPVAEIIMKPNVGQGSTSVQTAMESELGESSATINKRLCKSTIELSENSLLPASSMLTGTQSLLQPHLERVAIDALQLCCLLLPPPNRRKLQLLMRMISRMSQNVDMPKLHDAMGTRSLMIHTFSRCVLCCAEEVDLDELLAGRLVSFLMDHHQEILQVPSYLLDC

SEQ ID NO: 33 - DNA, 223 bp, Homo sapiens
gatctcactcagcagacagcagcagcccgggagcctgagctcaggaggaactcttacctggaaattgggaactgtatggagactccaaactgacttctttcaaaaaacaaaaacaaaaaatttttttagctttgacaaacacacaaaagtggtaataaagagagccctccttgtcaacccaaaatgtgagccccctgtggcaaaaccaccccctaccccatta

SEQ ID NO: 34 - DNA, 6543 bp, Homo sapiens
cggccgcggggcccggcgcggcgcgggccaaggagacggcgttcgtggaggtggtgctgttcgagtcgagcccaagcggcgattacaccacctacaccaccggcctcacgggccgcttctcgcgggccggggccacgctcagcgccgagggcgagatcgtgcagatgcacccactgggcctatgtaataacaatgacgaagaggacttgtatgaatatggctgggtaggagtggtgaagctggaacagccagaattggacccgaaaccatgcctcactgtcctaggcaaggccaagcgagcagtacagcggggagctactgcagtcatctttgatgtgtctgaaaacccagaagctattgatcagctgaaccagggctctgaagacccgctcaagaggccggtggtgtatgtgaagggtgcagatgccattaagctgatgaacatcgtcaacaagcagaaagtggctcgagcaaggatccagcaccgccctcctcgacaacccactgaatactttgacatggggattttcctggctttcttcgtcgtggtctccttggtctgcctcatcctccttgtcaaaatcaagctgaagcagcgacgcagtcagaattccatgaacaggctggctgtgcaggctctagagaagatggaaaccagaaagttcaactccaagagcaaggggcgccgggaggggagctgtggggccctggacacactcagcagcagctccacgtccgactgtgccatctgtctggagaagtacattgatggagaggagctgcgggtcatcccctgtactcaccggtttcacaggaagtgcgtggacccctggctgctgcagcaccacacctgcccccactgtcggcacaacatcatagaacaaaagggaaacccaagcgcggtgtgtgtggagaccagcaacctctcacgtggtcggcagcagagggtgaccctgccggtgcattaccccggccgcgtgcacaggaccaacgccatcccagcctaccctacgaggacaagcatggactcccacggcaaccccgtcaccttgctgaccatggaccggcacggggagcagagcctctattccccgcagacccccgcctacatccgcagctacccacccctccacctggaccacagcctggccgctcaccgctgcggcctggagcaccgggcctactccccagcccaccccttccgcaggcccaagttgagtggccgcagcttctccaaggcagcttgcttctcccagtatgagaccatgtaccagcactactacttccagggcctcagctacccggagcaggaggggcagtccccacctagcctcgcaccccggggcccggcccgtgcctttcctccgagcggcagtggcagcctgctcttccccaccgtggtgcacgtggccccgccctcccacctggagagcggcagcacgtccagcttcagctgctatcacggccaccgctcggtgtgcagtggctacctggccgactgcccaggcagcgacagcagcagcagcagcagctccggccagtgccactgttcctccagtgactctgtggtagactgcactgaggtcagcaaccagggcgtgtacgggagctgctccaccttccgcagctccctcagcagcgactatgaccccttcatctaccgcagccggagcccctgtcgtgccagtgaggcggggggctcgggcagctcgggccggggacctgccctgtgcttcgagggctccccgcctcccgaggagctcccggcggtgcacagtcatggtgctgggcggggcgagccttggccgggccctgcctctccctcgggggatcaggtgtccacctgcagcctggagatgaactacagcagcaactcctccctggagcacagggggcccaatagctctacctcagaagtggggctcgaggcttctcctggggccgcccctgacctcaggaggacctggaaggggggccacgagttgccgtcgtgtgcctgctgctgcgagccccagccctccccagccgggcctagcgccggagcagctggcagcagcaccttgttcctggggccccacctctacgagggctctggcccggcgggtggggagccccagtcaggaagctcccagggcttgtacggccttcaccccgaccatttgcccaggacagatggggtgaaatacgagggtctgccctgctgcttctatgaagagaagcaggtggcccgcgggggcggagggggcagcggctgctacactgaggactactcggtgagtgtgcagtacacgctcaccgaggaaccaccgcccggctgctaccccggggcccgggacctgagccagcgcatccccatcattccagaggatgtggactgtgatctgggcctgccctcggactgccaagggacccacagcctcggctcctggggtgggacgcgaggcccggataccccacggccccacaggggcctgggagcaacccgggaagaggagcgggctctgtgctgccaggctagggccctactgcggcctggctgccctccggaggaggcgggtgctgtcagggccaacttccctagtgccctccaggacactcaggagtccagcaccactgccactgaggctgcaggaccgagatctcactcagcagacagcagcagcccgggagcctgagctcaggaggaactcttacctggaaattgggaactgtatggagactccaaactgacttctttcaaaaaacaaaaacaaaaaatttttttagctttgacaaacacacaaaagtggtaataaagagagccctccttgtcaacccaaaatgtgagccccctgtggcaaaaccaccccctaccccattaacaaatcaacagacaaaattctccgagtcctttgcctcttttgataacatgttgttctgttttgtaaagtgtgtgtgcttggggttccgaggtgtgggattgagttctctgctttgtttttttttaagatattgtatgtaaatgtaaaaagttatttaaatatatattttaaagaaccctaactgccaacttttgctgaaaaagaaaaaaaaatcactgctgcattaaatgaaccacatcatgtgtagatactgttgtctccctgaagggagctcaggcctttgaaaagctcagggcttcacctgccttagaaaatgaaccagaaacttgaagtaaagctagttgataggggtacaggctctgaggagcagtgcaaaactgcctctttctttctcgtggcaaatcccaatgtacacgatttcaggtctcagacgccatgcctctccagcccacgcctttaggcaggtgatggcagcagctaggaatagggtgtacatgatccacagccctgcggagccaggtcaagccgctgctatgaaagctccagggtgatggggacgattctgcccagtgtcctcagtctgtcccctcaggtcatggtcccaagtgaaatgacagagttcacagccctggtcttggctgaggtccaggtcatagtaagggcatgttcttggggccctcgacctgaactctgaccctccgggcagggaagaggaggttgtcccctttggttgtcctggctttggagtcctttgcaaaaatattttgggccccctgccactggctgcagaaatggctcgacggggtgtgtggggacagacacccagaaggaatgtacttttgtggccttggtgtccgatggggctgggggagagtgctctccactgacccagcagcacacccatgtgcagtgcgcctgcatctgtgtgggggcagccacaccccttggctgctgcttccttgggctgcctttctgggggcatgtgactggacctacgaggtctgcactgagctccatttgaatgatacctttcctatcccatttcccccacggaagcaccgcttcagggttattcagtcctctgcctcatggctgaaattgctcatctcgtctgcagatgtctactatcctgtctacctaatgcactattatgtattgattctccatgagacagagagagagagagactatcagatagtttacacccaaagggtaggtttttgtatatttttccagccttttttattaaggggaaggggagagtttaaaaacccaaaccgttgtggttttaaggtgtttcatttttaaaagggagagagaatctatttaaagctatttcagatcagggattgtcatccttttttgtccaatgtattccttgttctttaaaaaaattttttttagaggaaactaatattagtctttgtgttcactaactcttctggtcacttgtatttatttattcattcattcatcagatatttgttgccatctgaaagaactggcccagtgggtctgaaagctcgcttgagaataggaaacttgagacctggccccctgtgggtaggagaacaaggaccacctgggttctccagtcttgaacgagaatctcactcttatcagaatgtttttcttaacctcagcgtatgatgaggaaatttacttatctctagctaggatttgacaaattccaacatcaaatgatcaaaacatttgccactgaggcttcactggtgagatccgttctccgtcctcgggtgcagtcccttgggggctgctcctcggactgcgccccgcacacctgttatcgagggtgtgagaagcgcctaagctggtgacatgtgatctgggacgccttcatttctcgggccaggagtagcagctgctaaggacagcagcttgcattgcgtggttttagggaagcagggtctggcttttaatatgaactgcaaaaagcagcttctcactgatatttttttgttgttgtttctggggggtttttttgttttgtttttaatgcctttgagtgcatattttcttcctcgtctgaaaccgaactcccaaagtggctttctttagccctggctggaaaaccacctctcaatagccttaagcaataaatagatgagtagagaatgtggcttcaactgggcttattaaagtaagtgtgtctagttttcacttgaacaagtgatagctgcagatggcgaaagaaacccatttaatttttgtagcttacaggtggtagaaacaaaaatgcaattttaaaaccttaaataccaaataccaaccattgccttttttttttttgagatggaattttgctcttgtcacccaggctggagtgcaatggcgcgatctcacctcactgcaacctctgcctcccgggtccaagtgattctcctgcctcagcctcccaagtagctgggattacaggcatgcgccaccacacccagctaattttgtatttttggtagagacagggtatctccatgttggtcaggctggtcttggattcccgacctcaggtgatccgcccacctcggcctcccaaagtgctgggattacaggcgtgagccaccatgcctgcccagcaataccaaccattgtcttttaaattcgtgttggcttctcagacagggagatcactggaataaaataaccgatggtcttattttgtcacacgtaaatcaaaagaaatgtcctctttgaagttgtaagactccaccaatgacagacacccttttcggtggactctgagtggtgtgtagtggttttatagccatggaaactaggagtatctcactttccactgagaacccctgcccccaatccctctaagttggggtgtggcagttgggcagggtcaagtgacccagccctggctgtaggacagccatatacagtgaagagttctagaaccagctaaaaatggaagtttgggtgtttaccaacaaggtacctctttatggatgcagccccagtaagctggctttaactctcagctccttccctgtctcctcctaatccaagcccttttataaaataaagccccttctgtcccactgctcacatacttatgtgctgctagtctctactcgaagttcgtgcaggactaatgcttttaaaatgaggtctaaaaaataattactagtcgagactattattctttaaacagaactgcctttttctactctttatgtaaactctttctattgtgttggtctaacaaggcactattttaaaattttttaatttttcccatagcacttaaaagagattttgtaaagaccttgctgtaaagattttgtaataaaatggtctaagggctctttttccaacattaccatttttaaaaaatgttttaaaagctagaagacaacttatgtatattctgtatatgtatagcagcacatttcatttatggaaatatgttctcagaatatttatttactaatatatttatcttaagccatgtcttatgttgagagtgtgacattgttggaataatcattgaaaatgactaacacaagaccctgtaaatacatgataattgcacacagattttacatatttgcagaccaaaaatgatttaaaacaagttgtagtcttctatggttttgtaacaaattgtacacatgactgtaaaaaaaaaatacaattttatcaagtatgtgttata

SEQ ID NO: 35 - protein, 836 aa, Homo sapiens
MHPLGLCNNNDEEDLYEYGWVGVVKLEQPELDPKPCLTVLGKAKRAVQRGATAVIFDVSENPEAIDQLNQGSEDPLKRPVVYVKGADAIKLMNIVNKQKVARARIQHRPPRQPTEYFDMGIFLAFFVVVSLVCLILLVKIKLKQRRSQNSMNRLAVQALEKMETRKFNSKSKGRREGSCGALDTLSSSSTSDCAICLEKYIDGEELRVIPCTHRFHRKCVDPWLLQHHTCPHCRHNIIEQKGNPSAVCVETSNLSRGRQQRVTLPVHYPGRVHRTNAIPAYPTRTSMDSHGNPVTLLTMDRHGEQSLYSPQTPAYIRSYPPLHLDHSLAAHRCGLEHRAYSPAHPFRRPKLSGRSFSKAACFSQYETMYQHYYFQGLSYPEQEGQSPPSLAPRGPARAFPPSGSGSLLFPTVVHVAPPSHLESGSTSSFSCYHGHRSVCSGYLADCPGSDSSSSSSSGQCHCSSSDSVVDCTEVSNQGVYGSCSTFRSSLSSDYDPFIYRSRSPCRASEAGGSGSSGRGPALCFEGSPPPEELPAVHSHGAGRGEPWPGPASPSGDQVSTCSLEMNYSSNSSLEHRGPNSSTSEVGLEASPGAAPDLRRTWKGGHELPSCACCCEPQPSPAGPSAGAAGSSTLFLGPHLYEGSGPAGGEPQSGSSQGLYGLHPDHLPRTDGVKYEGLPCCFYEEKQVARGGGGGSGCYTEDYSVSVQYTLTEEPPPGCYPGARDLSQRIPIIPEDVDCDLGLPSDCQGTHSLGSWGGTRGPDTPRPHRGLGATREEERALCCQARALLRPGCPPEEAGAVRANFPSALQDTQESSTTATEAAGPRSHSADSSSPGA

SEQ ID NO: 36 - DNA, 569 bp, Homo sapiens
  misc_feature (1)..(569): n is a, c, g, or t
ttgtcttctacgaccagctgaagcaagtgatgaatgcgtacagagtcaagccggccgtctttgacctgctcctggctgttggcattgctgcctacctcggcatggcctacgtggctgtccaggtgagcagtgcccaggctcagcacttcagcctcctctacaagaccgtccagaggctgctcgtgaaggccaagacacagtgacacagccacccccacagccggagcccccgccgctccacagtccctggggccgagcacgagttggnaggggaccctcttctcccgtcntgccntcgggttgcccgcctcctccagagacttnncaagggcccatcaccactggcctctgggcacttgtgctgagactctgggacccaggcagctgccaccttgtcaccatgagagaatttggggagtgcttgcatgctagccagcaggctcctgtctgggtgccacggggccagcattttggagggagcttccttccttccttcctggacaggtcgtcatgatggatgcactgactgaccgtctggggctcaggctggtgtgggatgcagccggccg

SEQ ID NO: 37 - DNA, 2070 bp, Homo sapiens
ccagagtttgtcttctacgaccagctgaagcaagtgatgaatgcgtacagagtcaagccggccgtctttgacctgctcctggctgttggcattgctgcctacctcggcatggcctacgtggctgtccagcacttcagcctcctctacaagaccgtccagaggctgctcgtgaaggccaagacacagtgacacagccacccccacagccggagcccccgccgctccacagtccctggggccgagcacgagtgagtggacactgccccgccgcgggcggccctgcagggacaggggccctctccctccccggcggtggttggaacactgaattacagagcttttttctgttgctctccgagactggggggggattgtttcttcttttccttgtctttgaacttccttggaggagagcttgggagacgtcccggggccaggctacggacttgcggacgagccccccagtcctgggagccggccgccctcggtctggtgtaagcacacatgcacgattaaagaggagacgccgggaccccctgcccgatcgcgcgcggcctccgcccaccgcctcctgccgcaaggggcctggactgcaggcctgacctgctccctgctccgtgtctgtcctaggacgtcccctcccgctccccgatggtggcgtggacatggttatttatctctgctccttcttgcctggaggagggcagtgccagccctggggttctgggattccagccctcctggagccttttgttccccatgtggtctcagtgacccgtccccctgacagtgggctcggggagctgcatcacccagccttccccttctccgactgcagggtctgatgtcatcattgacagcctttgcttcgtgggggcctggcagggcccctgcctccccgacccccgacccactgcaaatccccgttcccctgcactcctcttctcccagcccatccctccggcccctgtgcctctgcggccccagcccagctcccagggccgtcacctgcttggccctggcccagctccctgccctgagtcctgagccagtgcctggtgtttcctgggctcggtactgggcccccaggccatccaggctttgccacggccagttggtcctccctggggaactgggtgcgggtggagtactgggaggcaggaggtggcccggggaggccttgtggctcctcccctcgctcctcgccctgggcctcagcttcctcatcaatagaaaggatgtgttcggggtgggggcgtcaggtgagaacgtttgctgggaaggagaggacttggggcatggcctctggggccacccttcctggaactcagagaggaaggtccgggccctcgggaagccttggacagaaccctccaccccgcagaccaggcgtcgtgtgtgtgtgggagagaaggaggcccgtgttgagctcagggagaccccggtgtgtccgttcttagcaatataacctacccagtgcgtgccgagcaggcttggtggggaagggacttgagctgggcaagtcctggcctggcacccgcagccgtctcccttccgtggcccagggaggtgtttgctgtccgaaggacctgggccggcccatgggagcctggggttctgtccagataggaccagggggtctcactttggccaccagttcttcggccagcacctctgccctccagaacctgcagcctggaggggtgaggggacaaccacccctctttcctccaggttggcaggggaccctcttctcccgtctgccctgcgggttgcccgcctcctccagagacttgcccaagggcccatcaccactggcctctgggcacttgtgctgagactctgggacccaggcagctgccaccttgtcaccatgagagaatttggggagtgcttgcatgctagccagcaggctcctgtctgggtgccacggggccagcattttggagggagcttccttccttccttcctggacaggtcgtcatgatggatgcactgactgaccgtctggggctcaggctggtgtgggatgcagccggccgatgagaaaataaagccatattgaatgat

SEQ ID NO: 38 - protein, 62 aa, Homo sapiens
PEFVFYDQLKQVMNAYRVKPAVFDLLLAVGIAAYLGMAYVAVQHFSLLYKTVQRLLVKAKTQ

SEQ ID NO: 39 - DNA, 4008 bp, Homo sapiens
gcaaggtcacgtcctgtccccacctttcgcccctcaccctagctcccccaacgccaaagacaaggttaagaaagtgatatcgcgaaatagttttttaaagcattttattgcattttatgacttggagtttatgtgaaacctcaacggtattagccgaacagcctgccgcaccttccgggagttccagagtgggcctacaactcccacagggctccgcgagcgccggacggacggactacaattcccgacaggcagcgcggctggcggggcggttcgccgcggtgcccacaggacctcagggcgagtgcgggctgccccgcgcggcgcccgcaggaccccggcggctacccatgccgaggtgagtccgcgggagccgccgccgccgccgtcccgtcccagctgccgccccgcgcggccccgccgccggccaggatgctggaggaagcgggcgaggtgctggagaacatgctgaaggcgtcttgtctgccgctcggcttcatcgtcttcctgcccgctgtgctgctgctggtggcgccgccgctgcctgccgccgacgccgcgcacgagttcaccgtgtaccgcatgcagcagtacgacctgcagggccagccctacggcacacggaatgcagtgctgaacacggaggcgcgcacgatggcggcggaggtgctgagccgccgctgcgtgctcatgcggctactggacttctcctacgagcagtaccagaaggccctgcggcagtcggcgggcgccgtggtcatcatcctgcccagggccatggccgccgtgccccaggacgtcgtccggcaattcatggagatcgagccggagatgctggccatggagaccgccgtccccgtgtactttgccgtggaggacgaggccctgctgtctatctacaagcagacccaggctgcctccgcctcccagggctccgcctctgctgctgaagtactgctgcgcacggccactgccaacggcttccagatggtcaccagcggggtacagagcaaggccgtgagtgactggctgattgccagcgtggaggggcggctgacggggctgggcggagaggaccttcccaccatcgtcatcgtggcccactacgacgcctttggagtggccccctggctgtcgctgggcgcggactccaacgggagcggcgtctctgtgctgctggagctggcacgcctcttctcccggctctacacctacaagcgcacgcacgccgcctacaacctcctgttctttgcgtctggaggaggcaagtttaactaccagggaaccaagcgctggctggaagacaacctggaccacacagactccagcctgcttcaggacaatgtggccttcgtgctgtgcctggacaccgtgggccggggcagcagcctgcacctgcacgtgtccaagccgcctcgggagggcaccctgcagcacgccttcctgcgggagctggagacggtggccgcgcaccagttccctgaggtacggttctccatggtgcacaagcggatcaacctggcggaggacgtgctggcctgggagcacgagcgcttcgccatccgccgactgcccgccttcacgctgtcccacctggagagccaccgtgacggccagcgcagcagcatcatggacgtgcggtcccgggtggattctaagaccctgacccgtaacacgaggatcattgcagaggccctgactcgagtcatctacaacctgacagagaaggggacacccccagacatgccggtgttcacagagcagatgcagatccagcaggagcagctggactcggtgatggactggctcaccaaccagccgcgggccgcgcagctggtggacaaggacagcaccttcctcagcacgctggagcaccacctgagccgctacctgaaggacgtgaagcagcaccacgtcaaggctgacaagcgggacccagagtttgtcttctacgaccagctgaagcaagtgatgaatgcgtacagagtcaagccggccgtctttgacctgctcctggctgttggcattgctgcctacctcggcatggcctacgtggctgtccagcacttcagcctcctctacaagaccgtccagaggctgctcgtgaaggccaagacacagtgacacagccacccccacagccggagcccccgccgctccacagtccctggggccgagcacgagtgagtggacactgccccgccgcgggcggccctgcagggacaggggccctctccctccccggcggtggttggaacactgaattacagagcttttttctgttgctctccgagactggggggggattgtttcttcttttccttgtctttgaacttccttggaggagagcttgggagacgtcccggggccaggctacggacttgcggacgagccccccagtcctgggagccggccgccctcggtctggtgtaagcacacatgcacgattaaagaggagacgccgggaccccctgcccgatcgcgcgcggcctccgcccaccgcctcctgccgcaaggggcctggactgcaggcctgacctgctccctgctccgtgtctgtcctaggacgtcccctcccgctccccgatggtggcgtggacatggttatttatctctgctccttcttgcctggaggagggcagtgccagccctggggttctgggattccagccctcctggagccttttgttccccatgtggtctcagtgacccgtccccctgacagtgggctcggggagctgcatcacccagccttccccttctccgactgcagggtctgatgtcatcattgacagcctttgcttcgtgggggcctggcagggcccctgcctccccgacccccgacccactgcaaatccccgttcccctgcactcctcttctcccagcccatccctccggcccctgtgcctctgcggccccagcccagctcccagggccgtcacctgcttggccctggcccagctccctgccctgagtcctgagccagtgcctggtgtttcctgggctcggtactgggcccccaggccatccaggctttgccacggccagttggtcctccctggggaactgggtgcgggtggagtactgggaggcaggaggtggcccggggaggccttgtggctcctcccctcgctcctcgccctgggcctcagcttcctcatcaatagaaaggatgtgttcggggtgggggcgtcaggtgagaacgtttgctgggaaggagaggacttggggcatggcctctggggccacccttcctggaactcagagaggaaggtccgggccctcgggaagccttggacagaaccctccaccccgcagaccaggcgtcgtgtgtgtgtgggagagaaggaggcccgtgttgagctcagggagaccccggtgtgtccgttctttagcaatataacctacccagtgcgtgccgagcaggcttggtggggaagggacttgagctgggcaagtcctggcctggcacccgcagccgtctcccttccgtggcccagggaggtgtttgctgtccgaaggacctgggccggcccatgggagcctggggttctgtccagataggaccagggggtctcactttggccaccagttcttcggccagcacctctgccctccagaacctgcagcctggaggggtgaggggacaaccacccctctttcctccaggttggcaggggaccctcttctcccgtctgccctgcgggttgcccgcctcctccagagacttgcccaagggcccatcaccactggcctctgggcacttgtgctgagactctgggacccaggcagctgccaccttgtcaccatgagagaatttggggagtgcttgcatgctagccagcaggctcctgtctgggtgccacggggccagcattttggagggagcttccttccttccttcctggacaggtcgtcatgatggatgcactgactgaccgtctggggctcaggctggtgtgggatgcagccggccgatgagaaaataaagccatattgaatgatcg

SEQ ID NO: 40 - protein, 563 aa, Homo sapiens
MLEEAGEVLENMLKASCLPLGFIVFLPAVLLLVAPPLPAADAAHEFTVYRMQQYDLQGQPYGTRNAVLNTEARTMAAEVLSRRCVLMRLLDFSYEQYQKALRQSAGAVVIILPRAMAAVPQDVVRQFMEIEPEMLAMETAVPVYFAVEDEALLSIYKQTQAASASQGSASAAEVLLRTATANGFQMVTSGVQSKAVSDWLIASVEGRLTGLGGEDLPTIVIVAHYDAFGVAPWLSLGADSNGSGVSVLLELARLFSRLYTYKRTHAAYNLLFFASGGGKFNYQGTKRWLEDNLDHTDSSLLQDNVAFVLCLDTVGRGSSLHLHVSKPPREGTLQHAFLRELETVAAHQFPEVRFSMVHKRINLAEDVLAWEHERFAIRRLPAFTLSHLESHRDGQRSSIMDVRSRVDSKTLTRNTRIIAEALTRVIYNLTEKGTPPDMPVFTEQMQIQQEQLDSVMDWLTNQPRAAQLVDKDSTFLSTLEHHLSRYLKDVKQHHVKADKRDPEFVFYDQLKQVMNAYRVKPAVFDLLLAVGIAAYLGMAYVAVQHFSLLYKTVQRLLVKAKTQ

SEQ ID NO: 41 - DNA, 558 bp, Homo sapiens
  misc_feature (1)..(558): n is a, c, g, or t
ttcttcctgtgttacaattaccctgtttctgattactacagcccaacccgggcggacaccaccaccattctggctgccggggctggagtgaccataggattctggatcaaccatttcttccagcttgtatccaagcccgctgaatctctccctgttattcagaacatcccaccgntcaccacctacatgttagntttgggtctgaccaaatttgcagtgggaattgtgttgatcctcttggttcgtcagcttgtacaaaatctctcactgcaagtattatactcatggttcnaggtnggtcnccaggaacaaggaggccaggcggagactggagattgaagtgccttacaagtttgttacctacacatctgttggcatctgcgctacaacctttgtgccgatgcttcacaggtttctgggattaccctgagtctcaaacagttggaaactagcccactggacatgaaagccaagacataggaaagttattggtaggcaaatcttgacaacttatttttctttaacaacaacaaaaagtcatacggctgtcttgctact

SEQ ID NO: 42 - DNA, 4291 bp, Homo sapiens
gcttatgtacagaagtacgtcgtgaagaattatttctactattacctattccaattttcagctgctttgggccaagaagtgttctacatcacgtttcttccattcactcactggaatattgacccttatttatccagaagattgatcatcatatgggttttggtgatgtatattggccaagtggccaaggatgtcttgaagtggccccgtccctcctcccctccagttgtaaaactggaaaagagactgatcgctgaatatggaatgccatccacccacgccatggcggccactgccattgccttcaccctccttatctctactatggacagataccagtatccatttgtgttgggactggtgatggccgtggtgttttccaccttggtgtgtctcagcaggctctacactgggatgcatacggtcctggatgtgctgggtggcgtcctgatcaccgcactcctcatcgtcctcacctaccctgcctggaccttcatcgactgcctggactcggccagccccctcttccccgtgtgtgtcatagttgtgccattcttcctgtgttacaattaccctgtttctgattactacagcccaacccgggcggacaccaccaccattctggctgccggggctggagtgaccataggattctggatcaaccatttcttccagcttgtatccaagcccgctgaatctctccctgttattcagaacatcccaccactcaccacctacatgttagttttgggtctgaccaaatttgcagtgggaattgtgttgatcctcttggttcgtcagcttgtacaaaatctctcactgcaagtattatactcatggttcaaggtggtcaccaggaacaaggaggccaggcggagactggagattgaagtgccttacaagtttgttacctacacatctgttggcatctgcgctacaacctttgtgccgatgcttcacaggtttctgggattaccctgagtctcaaacagttggaaactagcccactggacatgaaagccaagacataggaaagttattggtaggcaaatcttgacaacttatttttctttaacaacaacaaaaagtcatacggctgtcttgctactaccagataaatgatgctgctgtgtgaaaggaagaactgtctcatagcggtcattggtcgtccgtggtggttggttgtgctacagttgaacccaggctaaagaccataatccggatctttaaaggcacacaccgcgccccccccccccccgcccggcccctgctcctctcgctgttgcacgggctttggatctagtcatgggctggcaggaattgtggcctggcttaggaatagctatgagccccactgggttctggagagccagtagagatggggtgatctgggaggctggaggtagagcctttcttttccgttacaaccttgcctagcatggagttaactgtgcctggttgggtggtaagatcactctgaaagaaagctcactgtgaagagatgaaaggtggaggcagagctgtgaggtcatggggaaaagcctgctttccttataagtcctgctgttcatgttggaataaggatctgctcttccttgtttccatgcattttgcaggattccaggtaccattaccacactcttctgacccatgaaaccaactggctgctcacacatcaccaaacaggttgggggttagccttcagcacaggtggatacatctgggattcactgagattcctgccctctcctgcttcctagtggtttgggacaggccctctgcccatcgtcagcagttttttgctttcatacaaacctggaaggcactggcatctgcctaggaaagtggatctgtgaagaacagatgaactcaatcctttctggagtctgacaaagaagggataggcttccttgacattgcctgtcctgacaaggcctccctgacattactcctccaatttcacagttaccttctgtaaatctattttctcatctactgaatagaatcaggcgccctttttgtcttcccacctcttatctcttggcaattttaaggggaattaatgcaagaacaactttagtgtctcttgggaaaacaagccaaccaaatacaaaacccattaagcctactagggtgagtcctcttaacatgggaaggcgatgattatgcaaacaccggagttccctcctcttcagttcctaagaataaagaacaggtatcaagaactttctttaaagttagtgtaactatagttaacaaagtatccattgaagtttagtgcctgtaggactgagccagtgctttatcaacccaacacatcatcaccatgtgcatactctagaaaaaaaaatagcttccttaaaagttacagaggctcttaacgtgttaaaaccgaaaaatcacatttttcttgatttcaaatatgttctacggccttactgttgggatgatatttagtatgtaacttagcattccaatttctcaagaatttttaggccgggtgcggtggctcatgcctgtaatcccagcactttgggaggccgaggtgggcggaccacgaggtcaggagatcgagaccatcctggctaacacggtaccccgtctctactgaaaatacaaaaaaattagccggacgtggtggagggcgcctgtagtcccagctactcaggaggctgaggcaggagaatggcgtgaacccggtgagcggagcttgcagtgagccgagattgcgccactgcactccagcctgggcgacagagcgagactctctcaaaaaaaaaaaaaaagaatttttagcaaaacatcctgtttttacttaaaattcttctcatatttattatagttagaaggcaaagatcaagatgacctgccgtttgactgcttttacatcaaactctgcccagtatttgcagcacaactcaggggaagggccttagcttacaggtactcccagccttcatctgcccctgcagagcagtggctgtcagccggatgcggcacttttctgtattttcatccacacagctgcccagccagagttcgcaacactggatatttacaccaaataattgtggttgacttgtctgaagccagctgacaaaaggatcagcttttcccacttgtattttttaaaaagagggattgtgatcattgtcacagagtgggtgctggcctctcatatatatgatatatatatatcattttatatatatatatatatcatatacataatttttactgctgtctctagttttaagtcccaacaataggaaggccgatcagctatattgatatatttaaggctgtacttaactaatttgggctgaggatgaatatatcagccacagcacattaaagaatgagccaaggatttgtcatggttggtcactttttaaagtatttgattactgcaactggagaatgaaaagtgtatattggtgacgccaacctcagtttctgagcactcctgctctgtggtgagaatcagacaaaaattcatcggggtgaaaaaggcattacctgattcacacccttgtcttgctagccctcttccattcatttctcacacagcactttgctctgttaaatcctctctctgtctcagaccattgcttgccccttcaaagggtatggttcaggctcctttcaagacatttggagtttctctctggggaaagagagccccctactggtttggcttcagtctaggtccaccatccctctcgatctggcatcttggagattaatttaaaaggcaagctcaccacaatgtaagcctatggtctggccaaccttgcttttgggaactgtgacaccaaagcccccaggactatctgcctctccaggagccagatagaatgacatgcctttttcctaattgtccacattccacccccaacccactgccactgtgggccaagccatccatcttgcaatcttcatctaaaacagctctcatttcatgccagttttgctcaaacctgcaccgtcacaagatattcagaagatgaaaacgtagaagacacccctgaattaaaaacacttacatagcagtggctggaattactccaaaacgtgcccagtgatcgcactgtaacatgggattttctcacccaaataggcaactcatgcttcctgagtgtaatcaaagcatgtggtgttttggggccatatgcaccaggtttctattttagaaaccttcagctgtcttgcttatgtactgtatgtaaatttattctttttaaaaatcacttttatttgattttgacttattaaatgctttaaaagccag

SEQ ID NO: 43 - protein, 326 aa, Homo sapiens
AYVQKYVVKNYFYYYLFQFSAALGQEVFYITFLPFTHWNIDPYLSRRLIIIWVLVMYIGQVAKDVLKWPRPSSPPVVKLEKRLIAEYGMPSTHAMAATAIAFTLLISTMDRYQYPFVLGLVMAVVFSTLVCLSRLYTGMHTVLDVLGGVLITALLIVLTYPAWTFIDCLDSASPLFPVCVIVVPFFLCYNYPVSDYYSPTRADTTTILAAGAGVTIGFWINHFFQLVSKPAESLPVIQNIPPLTTYMLVLGLTKFAVGIVLILLVRQLVQNLSLQVLYSWFKVVTRNKEARRRLEIEVPYKFVTYTSVGICATTFVPMLHRFLGLP

SEQ ID NO: 44 - DNA, 699 bp, Homo sapiens
atggcggccactgccattgccttcaccctccttatctctactatggacagataccagtatccatttgtgttgggactggtgatggccgtggtgttttccaccttggtgtgtctcagcaggctctacactgggatgcatacggtcctggatgtgctgggtggcgtcctgatcaccgcactcctcatcgtcctcacctaccctgcctggaccttcatcgactgcctggactcggccagccccctcttccccgtgtgtgtcatagttgtgccattcttcctgtgttacaattaccctgtttctgattactacagcccaacccgggcggacaccaccaccattctggctgccggggctggagtgaccataggattctggatcaaccatttcttccagcttgtatccaagcccgctgaatctctccctgttattcagaacatcccaccactcaccacctacatgttagttttgggtctgaccaaatttgcagtgggaattgtgttgatcctcttggttcgtcagcttgtacaaaatctctcactgcaagtattatactcatggttcaaggtggtcaccaggaacaaggaggccaggcggagactggagattgaagtgccttacaagtttgttacctacacatctgttggcatctgcgctacaacctttgtgccgatgcttcacaggtttctgggattaccctga

SEQ ID NO: 45 - protein, 232 aa, Homo sapiens
MAATAIAFTLLISTMDRYQYPFVLGLVMAVVFSTLVCLSRLYTGMHTVLDVLGGVLITALLIVLTYPAWTFIDCLDSASPLFPVCVIVVPFFLCYNYPVSDYYSPTRADTTTILAAGAGVTIGFWINHFFQLVSKPAESLPVIQNIPPLTTYMLVLGLTKFAVGIVLILLVRQLVQNLSLQVLYSWFKVVTRNKEARRRLEIEVPYKFVTYTSVGICATTFVPMLHRFLGLP

SEQ ID NO: 46 - DNA, 429 bp, Homo sapiens
  misc_feature (1)..(429): n is a, c, g, or t
cgccggcggtgcgtgtgggaaggcgtggggtgcggaccccggcccgacctcnccgtcccgcccgccgccttctgcgtcgcgggngcgggccggcggggtcctctgacgcggcagacagnccctcgctgtcgcctccagtggttgtcgacttgcgggcggcccccctccgcggcggtgggggtgccgtcccgccggcccgtcgtgctgccctctcnnggggggtttgcgcgagcgtcggctccgcctgggcccttgcggtgctcctggagcgctccgggttgtccctcaggtgcccgaggccgaacggtggtgtgtcgttcccgcccccggcgccccctcctccggtcgccgccgcggtgtccgcgcgtgggtcctgagggagctcgtcggtgtggggttcgaggcggtttgagtgagacgagacgagac

SEQ ID NO: 47 - DNA, 455 bp, Homo sapiens
accctatagctccttacgctgggaaagctggttttttaaaaaaataataataaaatatttaatcttattaagtgttcatttaaaatgcgtaatgctttggaaataatgggtaacagatagcgagaggatatgtttataaagtgagcatgttggtcccatttataaatatatgtatgatttataagcttttttaaaacaaagctcaaattgttggtatttttctaaaatgtgcacagctgtattttacatgaaggctctttctaatgggttgttatactgtactcaacattttggacagcacatgaagtctgccaatgtacttaataaaacatgactttgtttatttaaagtttcttgctgtgaaaaagaactccctacctgtgagttcctttatttataattcttgaaaccaaaatgtataatgtacagttttcacaactgtatctgctctaata

SEQ ID NO: 48 - DNA, 506 bp, Homo sapiens
  misc_feature (1)..(506): n is a, c, g, or t
gaagctccaaatgctctgggtttcagctcctctgtgctgtggacnctgactttggctcagaactccgatttagtacaaaaggctcatttttatttcaggggcactcttcctaaagcaaacctaataaatgaaatatggaattcacagatacacacacacattaaaaaattaacctagtgtatctgtgaggagtaggcagaaattcnctgtataaaagaatgcttcatttcatagagaatttgtgttaagattccattagatagtacatttctcaaagatttttgaggttgtatttgctttaccaaaacttggtttatgtaagtggaaaaagcatgttgcaaaataacttggtgtctatgattcagtttatgtaaaataataaatgtatgtaggaatacgtgtgttgaaagatgtacatcaatttgctaacaatggttatctctgacgtggtgggatttgagatgtgtttttctttttggttgtatttttctctattgtttgactta

SEQ ID NO: 49 - DNA, 3578 bp, Homo sapiens
ggcgaggtgctggagaacatgctgaaggcgtcttgtctgccgctcggcttcatcgtcttcctgcccgctgtgctgctgctggtggcgccgccgctgcctgccgccgacgccgcgcacgagttcaccgtgtaccgcatgcagcagtacgacctgcagggccagccctacggcacacggaatgcagtgctgaacacggaggcgcgcacgatggcggcggaggtgctgagccgccgctgcgtgctcatgcggctactggacttctcctacgagcagtaccagaaggccctgcggcagtcggcgggcgccgtggtcatcatcctgcccagggccatggccgccgtgccccaggacgtcgtccggcaattcatggagatcgagccggagatgctggccatggagaccgccgtccccgtgtactttgccgtggaggacgaggccctgctgtctatctacaagcagacccaggctgcctccgcctcccagggctccgcctctgctgctgaagtactgctgcgcacggccactgccaacggcttccagatggtcaccagcggggtacagagcaaggccgtgagtgactggctgattgccagcgtggaggggcggctgacggggctgggcggagaggaccttcccaccatcgtcatcgtggcccactacgacgcctttggagtggccccctggctgtcgctgggcgcggactccaacgggagcggcgtctctgtgctgctggagctggcacgcctcttctcccggctctacacctacaagcgcacgcacgccgcctacaacctcctgttctttgcgtctggaggaggcaagtttaactaccagggaaccaagcgctggctggaagacaacctggaccacacagactccagcctgcttcaggacaatgtggccttcgtgctgtgcctggacaccgtgggccggggcagcagcctgcacctgcacgtgtccaagccgcctcgggagggcaccctgcagcacgccttcctgcgggagctggagacggtggccgcgcaccagttccctgaggtacggttctccatggtgcacaagcggatcaacctggcggaggacgtgctggcctgggagcacgagcgcttcgccatccgccgactgcccgccttcacgctgtcccacctggagagccaccgtgacggccagcgcagcagcatcatggacgtgcggtcccgggtggattctaagaccctgacccgtaacacgaggatcattgcagaggccctgactcgagtcatctacaacctgacagagaaggggacacccccagacatgccggtgttcacagagcagatgcagatccagcaggagcagctggactcggtgatggactggctcaccaaccagccgcgggccgcgcagctggtggacaaggacagcaccttcctcagcacgctggagcaccacctgagccgctacctgaaggacgtgaagcagcaccacgtcaaggctgacaagcgggacccagagtttgtcttctacgaccagctgaagcaagtgatgaatgcgtacagagtcaagccggccgtctttgacctgctcctggccgttggcattgctgcctacctcggcatggcctacgtggctgtccagcacttcagcctcctctacaggaccgtccagaggctgctcgtgaaggccaagacacagtgacacagccacccccacagccggagcccccgccgctccacagtccctggggccgagcacgagtgagtggacactgccccgccgcgggcggccctgcagggacaggggccctctccctccccggcggtggttggaacactgaattacagagcttttttctgttgctctccgagactggggggggattgtttcttcttttccttgtctttgaacttccttggaggagagcttgggagacgtcccggggccaggctacggacttgcggacgagccccccagtcctgggagccggccgccctcggtctggtgtaagcacacatgcacgattaaagaggagacgccgggaccccctgcccgatcgcgcgcggcctccgcccaccgcctcctgccgcaaggggcctggactgcaggcctgacctgctccctgctccgtgtctgtcctaggacgtcccctcccgctccccgatggtggcgtggacatggttatttatctctgctccttcttgcctggaggagggcagtgccagccctggggttctgggattccagccctcctggagccttttgttccccatgtggtctcagtgacccgtccccctgacagtgggctcggggagctgcatcacccagccttccccttctccgactgcagggtctgatgtcatcgttgacagcctttgcttcgtgggggcctggcagggcccctgcctccccgacccccgacccactgcaaacccccgttcccctgcactcctcttctcccagcccatccctccggcccctgtgcctctgcggccccagcccagctcccagggccgtcacctgcttggccctggcccagctccctgccctgagtcctgagccagtgcctggtgtttcctgggctcggtactgggcccccaggccatccaggctttgccacggccagttggtcctccctggggaactgggtgcgggtggagtactgggaggcaggaggtggcccggggaggccttgtggctcctcccctcgctcctcgccctgggcctcagcttcctcatcaatagaaaggatgtgttcggggtgggggcgtcaggtgagaacgtttgctgggaaggagaggacttggggcatggcctctggggccacccttcctggaactcagagaggaaggtccgggccctcgggaagccttggacagaaccctccaccccgcagaccaggcgtcgtgtgtgtgtgggagagaaggaggcccgtgttgagctcagggagaccccggtgtgtccgttctttagcaatataacctacccagtgcgtgccgagcaggcttggtggggaagggacttgagctgggcaagtcctggcctggcacccgcagccgtctcccttccgtggcccagggaggtgtttgctgtccgaaggacctgggccggcccatgggagcctggggttctgtccagataggaccagggggtctcactttggccaccagttcttcggccagcacctctgccctccagaacctgcagcctggaggggtgaggggacaaccacccctctttcctccaggttggcaggggaccctcttctcccgtctgccctgtgggttgcccgcctcctccagagacttgcccaagggcccatcaccactggcctctgggcacttgtgctgagactctgggacccaggcagctgccaccttgtcaccatgagagaatttggggagtgcttgcatgctagccagcaggctcctgtctgggtgccacggggccagcattttggagggagcttccttccttccttcctggacaggtcgtcaggatggatgcactgactgaccgtctggggctcaggctggtgtgggatgcagccggccgatgagaaaataaagccatattgaatgataaaaaaaaaaaaaaaaaaa

SEQ ID NO: 50 - protein, 552 aa, Homo sapiens
MLKASCLPLGFIVFLPAVLLLVAPPLPAADAAHEFTVYRMQQYDLQGQPYGTRNAVLNTEARTMAAEVLSRRCVLMRLLDFSYEQYQKALRQSAGAVVIILPRAMAAVPQDVVRQFMEIEPEMLAMETAVPVYFAVEDEALLSIYKQTQAASASQGSASAAEVLLRTATANGFQMVTSGVQSKAVSDWLIASVEGRLTGLGGEDLPTIVIVAHYDAFGVAPWLSLGADSNGSGVSVLLELARLFSRLYTYKRTHAAYNLLFFASGGGKFNYQGTKRWLEDNLDHTDSSLLQDNVAFVLCLDTVGRGSSLHLHVSKPPREGTLQHAFLRELETVAAHQFPEVRFSMVHKRINLAEDVLAWEHERFAIRRLPAFTLSHLESHRDGQRSSIMDVRSRVDSKTLTRNTRIIAEALTRVIYNLTEKGTPPDMPVFTEQMQIQQEQLDSVMDWLTNQPRAAQLVDKDSTFLSTLEHHLSRYLKDVKQHHVKADKRDPEFVFYDQLKQVMNAYRVKPAVFDLLLAVGIAAYLGMAYVAVQHFSLLYRTVQRLLVKAKTQ

SEQ ID NO: 51 - DNA, 348 bp, Homo sapiens
ttcctcggaggggccgtggtgagtctccagtatgttcggcccagcgctcttcgcacccttctggaccaaagcgccaaggactgcagccaggagagagggggctcacctcttatcctcggcgacccactgcacaagcaggccgctctcccagacttaaaatgtatcaccactaacctgtgagggggacccaatctggactccttccccgccttgggacatcgcaggccgggaagcagtgcccgccaggcctgggccaggagagctccaggaagggcactgagcgctgctggcgcgaggcctcggacatccgcaggcaccagggaaagtctcctggggcgatctgtaaat

SEQ ID NO: 52 - DNA, 2684 bp, Homo sapiens
gcttccagcggacggcagcgcgcgagcattgccccccctgcaccacctcaccaagatggctactttgggacacacattccccttctatgctggccccaagccaaccttcccgatggacaccactttggccagcatcatcatgatctttctgactgcactggccacgttcatcgtcatcctgcctggcattcggggaaagacgaggctgttctggctgcttcgggtggtgaccagcttattcatcggggctgcaatcctggggacccccgtgcagcagctgaatgagaccatcaattacaacgaggagttcacctggcgcctgggtgagaactatgctgaggagtatgcaaaggctctggagaaggggctgccagaccctgtgttgtacctagctgagaagttcactccaagaagcccatgtggcctataccgccagtaccgcctggcgggacactacacctcagccatgctatgggtggcattcctctgctggctgctggccaatgtgatgctctccatgcctgtgctggtatatggtggctacatgctattggccacgggcatcttccagctgttggctctgctcttcttctccatggccacatcactcacctcaccctgtcccctgcacctgggcgcttctgtgctgcatactcaccatgggcctgccttctggatcacattgaccacaggactgctgtgtgtgctgctgggcctggctatggcggtggcccacaggatgcagcctcacaggctgaaggctttcttcaaccagagtgtggatgaagaccccatgctggagtggagtcctgaggaaggtggactcctgagcccccgctaccggtccatggctgacagtcccaagtcccaggacattcccctgtcagaggcttcctccaccaaggcatactgtaaggaggcacaccccaaagatcctgattgtgctttataacattcctccccgtggaggccacctggacttccagtctggctccaaacctcattggcgccccataaaaccagcagaactgccctcagggtggctgttaccagacacccagcaccaatctacagacggagtagaaaaaggaggctctatatactgatgttaaaaaacaaaacaaaacaaaaagccctaagggactgaagagatgctgggcctgtccataaagcctgttgccatgataaggccaagcaggggctagcttatctgcacagcaacccagcctttccgtgctgccttgcctcttcaagatgctattcactgaaacctaacttcacccccataacaccagcagggtgggggttacatatgattctcctatggtttcctctcatccctcggcacctcttgttttcctttttcctgggttccttttgttcttcctttacttctccagcttgtgtggccttttggtacaatgaaagacagcactggaaaggaggggaaaccaaacttctcatcctaggtctaacattaaccaactatgccacattctctttgagcttcagttcccaaatttgctacataagattgcaagacttgccaagaatcttgggatttatctttctatgccttgctgacacctaccttggccctcaaacaccacctcacaagaagccaggtgggaagttagggaatcaactccaaaacgctattccttcccaccccactcagctgggctagctgagtggcatccaggacgggggagtgggtgacctgcctcatcactgccacctaacgtccccctggggtggttcagaaagatgctagctctggtagggtccctccggcctcactagagggcgcccctattactctggagtcgacgcagagaatcaggtttcacagcactgcggagagtgtactaggctgtctccagcccagcgaagctcatgaggacgtgcgaccccggcgcggagaagccatgaaaattaatgggaaaaacagtttttaaaaaacaaaagaaaaaaaggtttatttacagatcgccccaggagactttccctggtgcctgcggatgtccgaggcctcgcgccagcagcgctcagtgcccttcctggagctctcctggcccaggcctggcgggcactgcttcccggcctgcgatgtcccaaggcggggaaggagtccagattgggtccccctcacaggttagtggtgatacattttaagtctgggagagcggcctgcttgtgcagtgggtcgccgaggataagaggtgagccccctctctcctggctgcagtccttggcgctttggtccagaagggtgcgaagagcgctgggccgaacatactggagactcaccacggcccctccgaggaagaggcacaggacgcctgtggcggtggggatcgaaagaaaggagggcatgtggagtcagggctatgttgcccaggctggtctcgaactctggcctcaaacgaccttcctgcctcgacctcccaaagtgctgggattacaggcgtgatgcccgggccttcttccatcttttggagcctaccccttgtgttacctcccgccacacacctctaatctgaattacatgaaacacggcaagacaccaaacccttctgagccccccacttttcatctgtaaaatggtcataacagtgcctgtttctgcgaactattgagaggggcaaatagggtaatagatgtgaattcattctgtaaactgg

SEQ ID NO: 53 - protein, 298 aa, Homo sapiens
MATLGHTFPFYAGPKPTFPMDTTLASIIMIFLTALATFIVILPGIRGKTRLFWLLRVVTSLFIGAAILGTPVQQLNETINYNEEFTWRLGENYAEEYAKALEKGLPDPVLYLAEKFTPRSPCGLYRQYRLAGHYTSAMLWVAFLCWLLANVMLSMPVLVYGGYMLLATGIFQLLALLFFSMATSLTSPCPLHLGASVLHTHHGPAFWITLTTGLLCVLLGLAMAVAHRMQPHRLKAFFNQSVDEDPMLEWSPEEGGLLSPRYRSMADSPKSQDIPLSEASSTKAYCKEAHPKDPDCAL

SEQ ID NO: 54 - DNA, 981 bp, Homo sapiens
atgaccctgtggaacggcgtactgcctttttacccccagccccggcatgccgcaggcttcagcgttccactgctcatcgttattctagtgtttttggctctagcagcaagcttcctgctcatcttgccggggatccgtggccactcgcgctggttttggttggtgagagttcttctcagtctgttcataggcgcagaaattgtggctgtgcacttcagtgcagaatggttcgtgggtacagtgaacaccaacacatcctacaaagccttcagcgcagcgcgcgttacagcccgtgtccgtctgctcgtgggcctggagggcattaatattacactcacagggaccccagtgcatcagctgaacgagaccattgactacaacgagcagttcacctggcgtctgaaagagaattacgccgcggagtacgcgaacgcactggagaaggggctgccggacccagtgctctacctggcggagaagttcacaccgagtagcccttgcggcctgtaccaccagtaccacctggcgggacactacgcctcggccacgctatgggtggcgttctgcttctggctcctctccaacgtgctgctctccacgccggccccgctctacggaggcctggcactgctgaccaccggagccttcgcgctcttcggggtcttcgccttggcctccatctctagcgtgccgctctgcccgctccgcctaggctcctccgcgctcaccactcagtacggcgccgccttctgggtcacgctggcaaccggtgaggaccgagagaatgggccccgggggctaagggtggagacaggattcacaccgggcgtcctgtgcctcttcctcggaggggccgtggccgggaagcagtgcccgccaggcctgggccaggagagctccaggaagggcactgagcgctgctggcgcgaggcctcggacatccgcaggcaccagggaaagtctcctggggcgatctgtaaa

SEQ ID NO: 55 - protein, 327 aa, Homo sapiens
MTLWNGVLPFYPQPRHAAGFSVPLLIVILVFLALAASFLLILPGIRGHSRWFWLVRVLLSLFIGAEIVAVHFSAEWFVGTVNTNTSYKAFSAARVTARVRLLVGLEGINITLTGTPVHQLNETIDYNEQFTWRLKENYAAEYANALEKGLPDPVLYLAEKFTPSSPCGLYHQYHLAGHYASATLWVAFCFWLLSNVLLSTPAPLYGGLALLTTGAFALFGVFALASISSVPLCPLRLGSSALTTQYGAAFWVTLATGEDRENGPRGLRVETGFTPGVLCLFLGGAVAGKQCPPGLGQESSRKGTERCWREASDIRRHQGKSPGAICK

SEQ ID NO: 56 - DNA, 20 bp, Artificial
  PCR primer
accacagtccatgccatcac

SEQ ID NO: 57 - DNA, 20 bp, Artificial
  PCR primer
tccaccaccctgttgctgta

SEQ ID NO: 58 - DNA, 21 bp, Artificial
  PCR primer
tcccacccgctgtacctgtgc

SEQ ID NO: 59 - DNA, 21 bp, Artificial
  PCR primer
cctgcagctggcctggtacct

SEQ ID NO: 60 - DNA, 36 bp, Artificial
  PCR primer
ggaagatctgttgaagtgcattgctgcagctggtag

SEQ ID NO: 61 - DNA, 24 bp, Artificial
  PCR primer
cgccatccgagccttgctagccag

SEQ ID NO: 62 - DNA, 20 bp, Artificial
  PCR primer
accacagtccatgccatcac

SEQ ID NO: 63 - DNA, 20 bp, Artificial
  PCR primer
tccaccaccctgttgctgta

SEQ ID NO: 64 - DNA, 21 bp, Artificial
  PCR primer
aatgcagtgctgaacacggag

SEQ ID NO: 65 - DNA, 23 bp, Artificial
  PCR primer
tctgcttgtagatagacagcagg

SEQ ID NO: 66 - DNA, 187 bp, Homo sapiens
gatcctgggacccctgggccgtgcctgccctccaccttgagtgccatactcccaacagctccaggtacccaccgggggatgtgcctgctcaggaaacctctttgctccacacagcatggggcttcagctgctggcccaaggccaggagcgctgggttctgcagcagggctcagcctcaggggcgtta

SEQ ID NO: 67 - DNA, 2722 bp, Homo sapiens
ccctcccgcgtccggccgcgcccgtcctcctggctgcagagagactaccggccaccgccgccgccgccgccgcgagctgtccctgcggcgcgtctgccttggcggagccgaccgcagtgcgctcaggcgtccggtgcgtccccagcctccgccccggcgcgggggcgacggactcgcgcgtgcgcagcgccggaggggcgcgggctgggaccccctagccagcgcgtgcgccgatcgagcgcagggcgatgggtgggcgccgggcgccgggcgccaggcagtgatgggccttcccgcgctgcggccccactgaggaggaggctcggggacagcaggagcacgggctgcccgcgcggtgcggaccatggcgttcctggccgggccgcgcctgctggactgggccagctcgccgccgcacctgcagttcaataagttcgtgctgaccgggtaccggcccgccagcagcggctcgggctgcctgcgcagcctcttctacctgcacaacgaactgggcaacatctacacgcacgggctggccctgctgggcttcctggtgctggtgccaatgaccatgccctggggtcagctgggcaaggatggctggctgggaggcacacattgcgtggcctgccttgcaccccctgcaggctccgtgctctatcacctctttatgtgccaccaagggggcagcgctgtgtacgcccggctcctcgccctggacatgtgtggggtctgccttgtcaacacccttggggccctgcccatcatccactgcaccctggcctgcaggccctggctgcgcccggctgccctggtgggctacactgtgttgtcgggtgtggccggctggcgtgctctcaccgccccctccaccagtgctcggctccgggcatttggatggcaggctgctgcccgcctactggtatttggggcccggggagtgggtctgggttcaggggctccaggctccctgccctgctacctgcgcatggacgcactggcgctgcttgggggactggtaaatgtagcccgtctgcccgagcgctggggacctggccgctttgactactggggcaactcccaccagatcatgcacctgctgagcgtgggctccatcctgcagctgcacgccggcgtcgtgcccgacctgctctgggctgcccaccacgcctgtccccgggactgagctgccatgccagcctgcccacagcagcctcctagagttagcaacaccaggtgttcctcccaactcgtctgcaaggggctggctccttggatgcttccagctcatgagatgtctcagcaggagccctgttcacccgttcttccctgtggactgacctcttccacccacgccgtggcgctccaacttccttccctgccttttccctccaagctcctattttactgtgtcagctggaaggaaacctttccctcttgggacctctttaccctctgtgacctgtggggttagaccagagagggactctggggtcacgtcttgctctgagagttcaagtcctgccaggccgccagcccagagcctcctcaccctatcctgttcctcccaccaggcctgtggccagtcttcctgatctccatctttctgccctgcataccagccctcccagcagccacaagcttgcccgccctggctccctctgcccagagactatggagtaaggcattcaggacaaaaggaccaagggggcgtggacccgtcttgtaccagctggccacaggcacaagggctgcagctgcttcttccaggaaactgacacagggagctcagcggcctcagatcctgggacccctgggccgtgcctgccctccaccttgagtgccatactcccaacagctccaggtacccaccgggggatgtgcctgctcaggaaacctctttgctccacacagcatggggcttcagctgctggcccaaggccaggagcgctgggttctgcagcagggctcagcctcaggggcgttaagaccctggatgacatcaataaagggacaggaagggccatgttgccacatgagcaagcttgggtgctcccaaggttcaaatactttttattagacacggccaggcagagaagaccatgggagttcccgaggggccccagctttcaagggcgacgggagagacacaggataaaaggttaaaagtgcagaggcagagtctggggctcaggttgggtctagggtgtcctcaaacaggctgaggaggttccgaggctcaaaggaggggaaggagccccgaggaggctctgagttgatgtcacttaggtccagggcatccctgggaggagagagtagtgacactcaggatccaaaagctagccctgcccaccccagcccctggacctgcttacctgggtgtgcacctgctccggggggtggaggtgctccccacagtccgggccaggacagcctcaggggagagtgaaggcctgcaggagggcaggcgagacaaggagggtgtccagggctagggagtgccggatgaaaccagctctgtccctgtgcaggctccaggctcccgcctgacaaacaggcagggagccacagtcagggacaataaaaacttggtgcactctgaaagcagcacttggacagccttcaaagtccttccatctggctgcactccaaggccccctctgtccttttcagaacacatggacttggaggcagatttgaaataaacttttagtaaatgtaa

SEQ ID NO: 68 - protein, 273 aa, Homo sapiens
MAFLAGPRLLDWASSPPHLQFNKFVLTGYRPASSGSGCLRSLFYLHNELGNIYTHGLALLGFLVLVPMTMPWGQLGKDGWLGGTHCVACLAPPAGSVLYHLFMCHQGGSAVYARLLALDMCGVCLVNTLGALPIIHCTLACRPWLRPAALVGYTVLSGVAGWRALTAPSTSARLRAFGWQAAARLLVFGARGVGLGSGAPGSLPCYLRMDALALLGGLVNVARLPERWGPGRFDYWGNSHQIMHLLSVGSILQLHAGVVPDLLWAAHHACPRD

SEQ ID NO: 69 - DNA, 2618 bp, Homo sapiens
ctggcgtcccctcccgcgtccggccgcgcccgtcctcctggctgcagagagactaccggccaccgccgccgccgccgccgcgagctgtccctgcggcgcgtctgccttggcggagccgaccgcagtgcgctcaggcgtccggtgcgtccccagcctccgccccggcgcgggggcgacggactcgcgcgtgcgcagcgccggaggggcgcgggctgggaccccctagccagcgcgtgcgccgatcgagcgcagggcgatgggtgggcgccgggcgccgggcgccaggcagtgatgggccttcccgcgctgcggccccactgaggaggaggctcggggacagcaggagcacgggctgcccgcgcggtgcggaccatggcgttcctggccgggccgcgcctgctggactgggccagctcgccgccgcacctgcagttcaataagttcgtgctgaccgggtaccggcccgccagcagcggctcgggctgcctgcgcagcctcttctacctgcacaacgaactgggcaacatctacacgcacggctccgtgctctatcacctctttatgtgccaccaagggggcagcgctgtgtacgcccggctcctcgccctggacatgtgtggggtctgccttgtcaacacccttggggccctgcccatcatccactgcaccctggcctgcaggccctggctgcgcccggctgccctggtgggctacactgtgttgtcgggtgtggccggctggcgtgctctcaccgccccctccaccagtgctcggctccgggcatttggatggcaggctgctgcccgcctactggtatttggggcccggggagtgggtctgggttcaggggctccaggctccctgccctgctacctgcgcatggacgcactggcgctgcttgggggactggtaaatgtagcccgtctgcccgagcgctggggacctggccgctttgactactggggcaactcccaccagatcatgcacctgctgagcgtgggctccatcctgcagctgcacgccggcgtcgtgcccgacctgctctgggctgcccaccacgcctgtccccgggactgagctgccatgccagcctgcccacagcagcctcctagagttagcaacaccaggtgttcctcccaactcgtctgcaaggggctggctccttggatgcttccagctcatgagatgtctcagcaggagccctgttcacccgttcttccctgtggactgacctcttccacccacgccgtggcgctccaacttccttccctgccttttccctccaagctcctattttactgtgtcagctggaaggaaacctttccctcttgggacctctttaccctctgtgacctgtggggttagaccagagagggactctggggtcacgtcttgctctgagagttcaagtcctgccaggccgccagcccagagcctcctcaccctatcctgttcctcccaccaggcctgtggccagtcttcctgatctccatctttctgccctgcataccagccctcccagcagccacaagcttgcccgccctggctccctctgcccagagactatggagtaaggcattcaggacaaaaggaccaagggggcgtggacccgtcttgtaccagctggccacaggcacaagggctgcagctgcttcttccaggaaactgacacagggagctcagcggcctcagatcctgggacccctgggccgtgcctgccctccaccttgagtgccatactcccaacagctccaggtacccaccgggggatgtgcctgctcaggaaacctctttgctccacacagcatggggcttcagctgctggcccaaggccaggagcgctgggttctgcagcagggctcagcctcaggggcgttaagaccctggatgacatcaataaagggacaggaagggccatgttgccacatgagcaagcttgggtgctcccaaggttcaaatactttttattagacacggccaggcagagaagaccatgggagttcccgaggggccccagctttcaagggcgacgggagagacacaggataaaaggttaaaagtgcagaggcagagtctggggctcaggttgggtctagggtgtcctcaaacaggctgaggaggttccgaggctcaaaggaggggaaggagccccgaggaggctctgagttgatgtcacttaggtccagggcatccctgggaggagagagtagtgacactcaggatccaaaagctagccctgcccaccccagcccctggacctgcttacctgggtgtgcacctgctccggggggtggaggtgctccccacagtccgggccaggacagcctcaggggagagtgaaggcctgcaggagggcaggcgagacaaggagggtgtccagggctagggagtgccggatgaaaccagctctgtccctgtgcaggctccaggctcccgcctgacaaacaggcagggagccacagtcagggacaataaaaacttggtgcactctgaaagcagcacttggacagccttcaaagtccttccatctggctgcactccaaggccccctctgtccttttcagaacacatggacttggaggcagatttgaaataaacttttagtaaatgtaagcctt

SEQ ID NO: 70 - protein, 234 aa, Homo sapiens
MAFLAGPRLLDWASSPPHLQFNKFVLTGYRPASSGSGCLRSLFYLHNELGNIYTHGSVLYHLFMCHQGGSAVYARLLALDMCGVCLVNTLGALPIIHCTLACRPWLRPAALVGYTVLSGVAGWRALTAPSTSARLRAFGWQAAARLLVFGARGVGLGSGAPGSLPCYLRMDALALLGGLVNVARLPERWGPGRFDYWGNSHQIMHLLSVGSILQLHAGVVPDLLWAAHHACPRD

SEQ ID NO: 71 - DNA, 2166 bp, Homo sapiens
atgcactgagctccgacctggggttgccagctttctctcccttgcgggggcgtcgaactcgcgcgtgcgcagcgcgtgagggaagggggccgggacctccttgctgacccgggcagggccaccggatagccggaggtgaatcgggatgagcttcccagcgctgcagctccactgagaaggaagcccaggcgcagagggtcgccggtcggccgcagtgcgtgaggccatggcattcctgaccgggcctcgtctcctggactgggctagctcgccgccgcacctgcagttcaataagttcgtattaaccggctaccggccggccagcagcggctcgggctgtctgcgcagccttttctacctacacaacgagctgggcaacatctacacacacgggctagccctgctgggcttcctggtgttggtgccaatgaccatgccctggagtcagctgggcaaggatggctggctaggaggtacacactgtgtggcttgcctggtgccccctgcagcctctgtgctgtatcacctcttcatgtgccaccaaggaggcagtcctgtgtacacccggctccttgccttggatatgtgtggagtctgccttgtcaacacccttggagccctgcccatcatccattgcactctggcctgcagaccgtggcttcgcccagctgccctgatgggttacactgcactgtcaggtgtagccggctggagagctctcactgccccctccaccagtgcccggcttcgagcctttggttggcaagctggggcccgcctgctggtgtttggggcccgtggagtggggctgggctcaggggctccaggctctctgccctgctacctgcgcatggacgcactggctctgcttggagggctggtgaatgtggcacgcctgccagagcggtgggggcctggtcgcttcgactactggggcaactcccaccagatcatgcacttgctgagtgtgggctccatcctccagctccatgctggggttgtgcctgacctgctctgggctgcacaccatgcctgtcccccagactgagctgcctcctagctgccaaactggcttgcccacagcttcctggacaaattccaccacctttcctcctactggtctgcaaggggctggttccctggaagaaccagcacatgggacttcctagctgggagaccattcttcattcttccccatggattcacttcttgcatccaggccttcaaaccccagcttccactttccttgccatcttccctcctgggcattgttttgctgtcattagaaggaaaccatttttttttttcccaatttaccctgtttaacctgtgagagtctctgacagttgagtcctgccaacttaccaagcctccagcccagaaccactacccctatgttgctgctcccatacataactacacctcctgctcctggattcttgagctagccactctgaccctgcttcctgacctccatctccctgctctgcatgtcaaacctctcagcagccagaattttgctgttcctgtcattcctgcagtgaggatgcagaggagtgggaccaggcttctctcagagccaagtggacattggtcctgcttgtatcatctggccaggagacaggaggggaactgctgcttttcctaggcaacaggcacagctgtggaatggaggtgttggattcgggcttcactggaccaaggactcagctcttcagtgccatggtctgactgacctgcctaccagagacttgtctgctcaggaaatctctatacagtgggtggctccagcctgctggcccaagggtactgactcgcagccagatcatcccaaaggcccaagaccctaggcaacatcaataaagggacaagaagagctatgctgccacatgagcaaccttgggtgttcccaagacgcattactttttattagacacggaagtttcaggggagaggtgggcaagacggtcagaggtttaaaagcaccaaggctggctgggcctgtgctcaggctgggtctagggagtcctcaaacaggctgaggaggttccttggctcaaaggtggggcagggacctcttggaggctctgagtccacatcagttaggtccagggcatcccttgggggaggaagaagaagaaaaaaaaaaaaaaaaaaaaggccaca

SEQ ID NO: 72 - protein, 273 aa, Homo sapiens
MAFLTGPRLLDWASSPPHLQFNKFVLTGYRPASSGSGCLRSLFYLHNELGNIYTHGLALLGFLVLVPMTMPWSQLGKDGWLGGTHCVACLVPPAASVLYHLFMCHQGGSPVYTRLLALDMCGVCLVNTLGALPIIHCTLACRPWLRPAALMGYTALSGVAGWRALTAPSTSARLRAFGWQAGARLLVFGARGVGLGSGAPGSLPCYLRMDALALLGGLVNVARLPERWGPGRFDYWGNSHQIMHLLSVGSILQLHAGVVPDLLWAAHHACPPD

SEQ ID NO: 73 - DNA, 6 bp, Artificial
  polyadenylation site
aataaa